(19) United States
(12) Patent Application Publication
(10) Pub. No.: US 2024/0335515 A1
(43) Pub. Date: Oct. 10, 2024

(54) ENZYME-LOADED POLYMERIC NANOPARTICLES AND METHODS OF MANUFACTURE AND USE OF SAME

(71) Applicant: University of Washington, Seattle, WA (US)

(72) Inventors: ELIZABETH NANCE, Seattle, WA (US); ANDREA JOSEPH, Seattle, WA (US); CHRIS NYAMBURA, Seattle, WA (US); WALTER JAMES PFAENDTNER, Seattle, WA (US); THOMAS WOOD, Seattle, WA (US)

(73) Assignee: University of Washington, Seattle, WA (US)

(21) Appl. No.: 18/577,620

(22) PCT Filed: Jul. 12, 2022
(86) PCT No.: PCT/US22/73650

§ 371 Date: Jan. 8, 2024

Related U.S. Application Data
(60) Provisional application No. 63/221,827, filed on Jul. 14, 2021.

(51) Int. Cl. A61K 38/44; A61K 47/54; A61K 47/61; A61P 25/28; C12N 9/08; C12N 11/08

(52) U.S. Cl. CPC A61K 38/44; A61K 47/541; A61K 47/61; A61P 25/28; C12N 9/0065; C12N 11/08; C12Y 111/01006

(57) ABSTRACT

Enzyme-loaded polymeric nanoparticles for the treatment of disorders such as neurological or non-neurological conditions are described. Using hydrophobic ion pairing, enzymes are loaded into polymeric nanoparticles while retaining enzymatic activity. Surfactants coating the nanoparticle can direct nanoparticles for cellular uptake or for aggregation in the extracellular matrix. Preparing a hydrophobic ion pairing complex for nanoparticle formation.

### CROSS-REFERENCE TO RELATED APPLICATIONS

This application is a U.S. National Phase based on International Patent Application No. PCT/US2022/073650, filed on Jul. 12, 2022, which claims priority to U.S. Provisional Patent Application No. 63/221,827 filed Jul. 14, 2021, each of which is incorporated herein by reference in its entirety as if fully set forth herein.

### STATEMENT REGARDING FEDERALLY SPONSORED RESEARCH OR DEVELOPMENT

This invention was made with government support under Grant No. HD100639, awarded by the National Institutes of Health, and Grant No. 1703438, awarded by the National Science Foundation. The government has certain rights in the invention.

### FIELD OF THE DISCLOSURE

The current disclosure describes enzyme-loaded polymeric nanoparticles for the treatment of disorders such as acute neurological injury or chronic neurodegenerative disorders. Methods to form the enzyme-loaded nanoparticles by forming or utilizing hydrophobic ion complexes are also described.

### BACKGROUND OF THE DISCLOSURE

Neonatal hypoxic-ischemic encephalopathy (HIE) is a devastating neurological condition that affects 1.3-4.7 in 1000 live births in the United States. Without treatment, ⅔ of affected infants with this condition will die or have a serious neurological disability, like cerebral palsy, seizures, intellectual disability, or developmental delays. The current standard-of-care, therapeutic hypothermia (TH), is only clinically implemented in cases of moderate or severe HIE and still leaves more than 30% of infants dead or with severe disability. One strategy to improve neonatal outcomes is to use a pharmaceutical agent to complement the neuroprotective mechanisms of TH. For example, catalase, a large (240 kDa) enzyme converts the reactive oxygen species (ROS) hydrogen peroxide to water and molecular oxygen and subsequently suppresses inflammation. For neurological applications, catalase can attenuate neuroinflammatory and apoptotic pathways in vitro, but its application in vivo has been limited due to its short half-life, proteolytic degradation, immunogenicity, and inability to penetrate the brain.

Nanoparticles serve as vehicles that can improve drug biodistribution and bioavailability. Drug-loaded biodegradable nanoparticles composed of poly(lactic-co-glycolic acid)-poly(ethylene glycol) (PLGA-PEG) have been shown to improve drug solubility, stability, circulation time, release kinetics, and transport to and within the brain parenchyma. Enzymes can particularly benefit from nanoparticle encapsulation as the polymer matrix provides protection from immune clearance and systemic degradation. However, the hydrophilic nature of enzymes limits their encapsulation into the hydrophobic core of PLGA-PEG nanoparticles. Additional challenges stem from the use of solvents during polymer nanoparticle manufacturing that denature proteins and the difficulty of retaining bioactivity following encapsulation into polymeric nanoparticles.

### SUMMARY OF THE DISCLOSURE

The present disclosure describes enzyme-loaded polymeric nanoparticles wherein the lipophilicity of the enzyme is increased via hydrophobic ion pairing. In this technique, complexes are formed by electrostatic interactions between ionizable groups on the enzyme and an ion-pairing agent (hydrophobic ion). Because of the hydrophobicity of the resulting enzyme-hydrophobic ion complex (hydrophobic ion pairing complex), the enzyme can be loaded into a polymeric nanoparticle while retaining its activity.

In particular embodiments, an enzyme-loaded polymeric nanoparticle includes a hydrophobic ion pairing complex encapsulated within a polymer, wherein the hydrophobic ion pairing complex includes a hydrophobic ion and an enzyme. In particular embodiments, the polymer includes a hydrophobic polymer and polyethylene glycol. In particular embodiments, the hydrophobic polymer includes polylactic co-glycolic acid (PLGA), polylactic acid (PLA), polyglycolic acid (PGA), poly-ϵ-caprolactone (PCL), ethyl cellulose (EC), polybutylcyanoacrylate (PBCA), polypropylene oxide (PPO), or polyhydroxybutyrate (PHB). In particular embodiments, the hydrophobic polymer includes PLGA. In particular embodiments, the hydrophobic ion includes dextran sulfate. In particular embodiments, the enzyme retains activity within the nanoparticle. In particular embodiments, the enzyme treats a neurological condition including an acute neurological injury or a chronic neurodegenerative condition. In particular embodiments, the enzyme treats a non-neurological condition including an oxidative stress burden, a lysosomal storage disease, or a metabolic deficiency. In particular embodiments, the enzyme is catalase or lysozyme.

The present disclosure includes an enzyme-loaded polymeric nanoparticle, methods of preparing a hydrophobic ion pairing complex, methods of preparing the enzyme-loaded polymeric nanoparticle, and methods of treating a condition with the enzyme-loaded polymeric nanoparticle.

### BRIEF DESCRIPTION OF THE FIGURES

Some of the drawings submitted herewith may be better understood in color. Applicant considers the color versions of the drawings as part of the original submission and reserves the right to present color images of the drawings in later proceedings.

FIG. 1. Graphical abstract of embodiments of the disclosure.

FIG. 2. Diagram of disease pathway in hypoxic ischemic encephalopathy (HIE). During a hypoxic-ischemic insult, the brain suffers from a period of no oxygen or blood flow, which are the essential materials needed for energy production. Without energy, brain cells can swell and die, but they can also fail in other ways: ion transporters fail, causing neuronal depolarization and extensive release of glutamate, one of the brain's main neurotransmitters. When glutamate activates downstream neurons, it causes a large influx of calcium, which goes on to trigger the production of nitric oxide and many other reactive oxygen species. These molecules are known for their toxicity to lipid membranes and DNA, which exacerbates long-term damage.

FIGS. 3A-3C. (3A) Polymeric nanoparticles can be used to overcome enzyme delivery challenges. The diagram shows a copolymer of poly(lactic-co-glycolic acid), conjugated to poly(ethylene glycol)—both of which are biodegradable and FDA-approved components. (3B) Fluorescent images shows that 50 nm nanoparticles can distribute through the injured brain parenchyma. (3C) polylactic co-glycolic acid (PLGA)-polyethylene glycol (PEG) nanoparticles can internalize within microglia and neurons.

FIGS. 4A-4F. Characterization of dextran sulfate (DS)-catalase (CAT) complexation under various formulation conditions. (4A) taurocholic acid (TA), (4B) sodium dodecyl sulfate (SDS), and (4C) DS demonstrated an increasing trend of catalase binding efficiency with respect to increasing molar ratio. For DS-CAT complexes, more acidic pHs are associated with (4D) increased binding efficiency but (4E) increased loss of activity. (4F) Binding efficiency in citrate buffer increases with increasing molar ratio. Values are represented as mean±standard deviation (SD) (n=3).

FIG. 5. Bovine serum albumin (BSA) is well matched to catalase, having a similar profile of surface amino acid residues. Surface amino acid composition of BSA at pH 3.7 (left bar in each pair) in its native state, compared to catalase at pH 4.7 (right bar in each pair).

FIGS. 6A-6C. Characterization of BSA structure and ion pairing agent interactions with surface amino acids. (6A) BSA backbone root mean squared deviation (RMSD) (in nanometers [nm]) vs. time (in nanoseconds[ns]), (6B) Backbone RMSD of each BSA domain vs time, (6C) Fraction of surface residues with >95% occupancy vs. residue grouping.

FIG. 7. Amino acid grouping used in FIG. 6C (for residues with >95% occupancy).

FIG. 8. Snapshots of the final structures of each of the simulations. End of trajectory snapshots (t=200 ns) of BSA without (top row) and with (bottom row) ion-pairing (IP)agents and neutralizing ions. Chlorine ions (CI) are present surrounding BSA in the BSA/TA, BSA/SDS, and BSA/DS complexes. Potassium ions are present surrounding BSA in the BSA/CI complex.

FIG. 9. Nanoparticles were characterized in terms of hydrodynamic diameter, mean surface charge (ζ-potential), and the polydispersity index (PDI) by dynamic light scattering at 25° C. and pH 7.2 in 10 mM NaCl. All values are reported as mean±standard error of the mean (SEM) (n=3).

FIG. 10. Diagram of catalase nanoparticle formulation methods. First, the enzyme (e.g., catalase) is mixed with the hydrophobic ion in order to increase hydrophobicity while maintaining its structure and function. Hydrophobic ion pairing (HIP) complexes were then dissolved into an organic solvent (e.g., water miscible organic solvent) with the PLGA-PEG polymer and the solution was added dropwise into an aqueous sink, where nanoparticles formed spontaneously. Finally, the nanoparticles were collected by centrifugation and washed with deionized (DI) water.

FIGS. 11A-11D. Catalase loading and protection in PLGA-PEG nanoparticles by formulation method. (11A) Nanoprecipitation and emulsion nanoparticles achieve non-significantly different catalase loading by activity, but (11B) nanoprecipitation nanoparticles have significantly lower catalase loading by mass (p=0.0003). (11C) Both methods result in retention of catalase activity over 24 h in 0.2% pronase solution. Values are represented as mean±SD (n=4 for A and B; n=3 for C). (11D) Catalase-loaded nanoparticles were incubated in a protease solution for 24 h to replicate the degradative conditions of in vivo circulation. A retention of nanoparticle catalase activity was observed through this period, with the nanoparticles exhibiting around 20% of their initial activity 24 h later. This result is in contrast to free catalase, which immediately deactivates upon introduction to a protease solution.

FIGS. 12A-12C. Global brain injury is significantly reduced by treatment with catalase-loaded nanoparticles. (12A) Rats were injured at P10 (day 10), received treatment 30 min, 24 h, and 48 h after injury, and were sacrificed at 72 h for endpoint analysis. (12B) Median (IQR) gross injury scores in the saline, blank nanoparticle, and catalase (CAT) nanoparticle groups are 0.5 (0-3), 2 (0-3.5), and 0 (0-2). Gross injury scores assess the appearance of the whole brain. The catalase treatment group had significantly lower injury severity compared to the saline group. (12C) Median (interquartile range, IQR) area loss measurements in the saline, blank nanoparticle, and catalase nanoparticle groups are 13% (10-31%), 23% (16-41%), and 4.9% (0.61-27%). For both assessments, treatment with catalase is significantly neuroprotective compared to saline (p=0.039 and p=0.047, respectively) while blank nanoparticles have no significant effect.

FIGS. 13A, 13B. (13A) Representative images of gross injury scoring and area loss measurements. Injury in 10 day old Vannucci model rats were induced by a surgical ligation of the left carotid artery, followed by 2 h incubation in a low oxygen chamber. The pups were randomly assigned into three treatment groups: saline, a blank nanoparticle loaded with the protein Bovine Serum Albumin, and the catalase-loaded nanoparticles. Treatments were administered 30 min, 24 h, and 48 h after injury, and assessed outcomes 72 h after injury. Global injury was measured using two techniques—gross injury scoring, looking at edema and tissue integrity of the whole brain, and area loss, which provides more regional insight. Immunofluorescence staining and confocal imaging was also conducted to look at nanoparticle distribution and cellular changes in injured tissue. (13B) Representative gross injury and area loss images from each group. In the saline and PLGA-PEG/BSA groups, the left hemisphere (left side of each slice) is noticeably smaller than the right hemisphere.

FIGS. 14A, 14B. Confocal images of nanoparticle distribution and microglial response to treatment in the ipsilateral hemisphere. (14A) PLGA-PEG nanoparticles are observed in the cortex, dentate gyrus, and midbrain of the injured hemisphere, but appear trapped in the vasculature of the contralateral hemisphere. Neurons and cell nuclei are also shown. Scale bars: 50 μm. In the ipsilateral hemisphere, distinct spots of nanoparticles are observed throughout the brain tissue as indicated by the arrows. (14B) Microglia have increased number and density in the ipsilateral compared to contralateral hemisphere in saline- and blank nanoparticle-treated pups. After catalase nanoparticle treatment, microglia number and density appear similar between hemispheres. Scale bars: 300 μm.

FIG. 15. Graphical overview of nanoparticle formulation, nanoparticle components, and accumulation in the brain.

FIG. 16. Physicochemical properties and diffusivity of PLGA-PEG nanoparticles formulated with biocompatible surfactants. Nanoparticles were characterized in terms of hydrodynamic diameter, mean surface charge (ζ-potential), and the polydispersity index (PDI) by dynamic light scattering at 25° C. and pH 7.2 in 10 mM NaCl. All values are reported as mean±standard error of the mean (SEM) (n=3). Effective diffusion coefficients in rat brain tissue (D_{b}) were extracted at a T=0.8 s from nanoparticle trajectories over fifteen videos across three brain slices for each formulation, and compared to theoretical nanoparticle diffusion in artificial cerebrospinal fluid (aCSF) based on the Stokes-Einstein equation and mean particle diameter.

FIG. 17. Extracted ion chromatograms for quantification of surfactants in nanoparticle suspensions. Surfactants polysorbate 80 (P80) and cholic acid (CHA) were quantified by comparison of peak area to a calibration curve of known standards.

FIGS. 18A,18B. Ex vivo uptake of nanoparticles and cytotoxicity by lactate dehydrogenase. (18A) All nanoparticle formulations are observed in neurons (left) and microglia (right). All cell nuclei are stained with DAPI (4′,6-diamidino-2-phenylindole) and all scale bars represent 20 μm. (18B) Cytotoxicity is within a normal 5-25% range for all nanoparticle treatment conditions.

FIGS. 19A-19D. Assessment of surfactant effects on nanoparticle transport in organotypic brain slices. (19A) Flow cytometry analysis indicate that all formulations with surfactant demonstrated similar levels of microglial uptake within 4 h, while PLGA-PEG/deionized water (DI) achieved significantly elevated levels of uptake. (19B) Propidium iodide (PI)-positive cell counts, as a proportion of total cells, demonstrate no significant differences in cytotoxicity across all treatment conditions. (19C) Nanoparticle trajectories were analyzed to calculate ensemble-averaged mean squared displacement at time lags up to 6.5 s. (19D) Log of D_{b} at 0.8 s were extracted for each trajectory (1 dot=1 trajectory).

FIGS. 20A-20D. Geometric analysis of nanoparticle diffusion trajectories. (20A) The aspect ratio of the mean-square displacement (MSD) curve for each trajectory was extracted and classified as subdiffusive (<0) or superdiffusive (>0). (20B) The anomalous exponent a was extracted for each trajectory and classified as superdiffusive (>1), normal (1), or subdiffusive (<1). The (20C) trappedness and (20D) efficiency of each trajectory was calculated and plotted as violin plots.

FIGS. 21A-21D. Distribution of biodegradable, PEGylated nanoparticles (red) in the brain and major organs at t=4 h. (21A) PLGA-PEG/P80 nanoparticles, unlike all other formulations, exhibit significantly higher accumulation (p=0.0280) in the brain parenchyma (left bars, solid fill) compared to brain capillaries (right bars, hashed). (21B) PLGA-PEG/P80 nanoparticles can internalize within some microglia (green, top) and neurons (green, bottom) in the brain parenchyma. (21C) PLGA-PEG/DI, PLGA-PEG/CHA, PLGA-PEG/F68, PLGA-PEG/F127, and PLGA-PEG/PVA nanoparticles do not exhibit patterns of microglial (top row) or neuronal (bottom row) uptake, and instead appear associated within the vasculature. F68 stands for poloxamer 188, F127 stands for poloxamer 407, and PVA stands for polyvinyl alcohol. (21D) Nanoparticles demonstrate accumulation in the serum, liver, and spleen, with minimal signal from the kidney, heart, and lungs. (21A, 21D): Each dot represents one pup for a total of n=4 (brain) or n=5 (major organs). (21B-21C): All cell nuclei (blue) are stained with DAPI and all scale bars represent 20 μm.

FIGS. 22A-22D. Additional in vivo biodistribution images. (22A) Staining of ZO1 (green) confirms nanoparticle localization within blood vessels. (22B) PLGA-PEG/PVA nanoparticles appear stuck within blood vessel walls at both 4 h and 24 h post-administration. (22C) No microglial uptake of PLGA-PEG/PVA or PLGA/F127 nanoparticles was observed 24 h after administration. (22D) No neuronal uptake of PLGA-PEG/PVA or PLGA/F127 nanoparticles was observed 24 h after administration.

FIGS. 23A-23D. PLGA-PEG/P80 surface analysis and serum protein adsorption. Positive ion (23A) and negative ion 23(B) peak ratio from PDG-PEG/DI and PLGA-PEG/P80 Time-of-Flight secondary ion mass spectrometry (ToF-SIMS) data. (23C) Compared to the PLGA-PEG/DI control, PLGA-PEG/P80 nanoparticles exhibited increased protein adsorption (p=0.0382). (23D) PLGA-PEG/P80 demonstrated a negative shift in ζ-potential after plasma incubation, which was not observed with the PLGA-PEG/DI control.

FIG. 24. Proposed chemical identifications of selected ToF-SIMS peaks from nanoparticle and P80 control samples.

FIGS. 25A, 25B. Nanoparticle size and surface charge after 4-hour incubation in plasma. (25A) All formulations except PLGA-PEG/DI and PLGA/F127 maintain stability and low polydispersity after plasma incubation. (25B) PLGA-PEG/P80 and PLGA/F127 show the largest negative shifts in surface charge after plasma incubation.

FIG. 26. Quantification of adsorbed protein after 4-hour incubation in plasma. Only PLGA-PEG/P80 and PLGA-PEG/PVA formulations demonstrated significantly enhanced protein adsorption relative to the PLGA-PEG/DI control (p=0.038 and 0.025, respectively).

FIGS. 27A-27D. Representative FACS (flow cytometry) data from control samples. (27A) Based on forward and side scatter, gate P1 was drawn such that cellular debris would be eliminated. From height and width scatter, gate P2 was drawn to isolate single cells only. (27B) Gate P3 was drawn to isolate DAPI-negative (live) cells; data from an unstained sample (left) and DAPI-stained sample (right) are shown. (27C) Gate P4 was drawn to isolate CD11b-positive cells (microglia); data from an unstained sample (left) and CD11b-stained sample (right) are shown. (27D) Marker P6 was drawn to determine the proportion of microglia with nanoparticle fluorescence; data from a sample without nanoparticles (left) and with nanoparticles (right) are shown.

FIG. 28. Biodistribution calibration curves. Individual calibration curves for each tissue fraction-nanoparticle combination were created in order to accurately calculate nanoparticle accumulation in the brain.

FIGS. 29A, 29B. Catalase complexation with HIP agent (referred to herein as hydrophobic ion), dextran sulfate. HIP complexes were made using catalase (MW 240 kDa) and dextran sulfate (DS) at varying molar ratios (DS/Cat Molar ratio (MR)=1, 3, 5, 6, 10, 13). Catalase only with no DS was used as a control. (29A) The state point diagram demonstrates the range of pH's at which insoluble precipitates form (● (black circle): precipitate with clear supernatant and higher volume than ▪ (black square): precipitate with clear solution; ▴ (black triangle): precipitate with cloudy solution) indicating formation of a successful hydrophobic ion complex. □ (white square): represents a turbid solution; ∘ (white circle): represents a translucent solution (no precipitate). (29B) Representative visuals of the various conditions.

FIGS. 30A-30D. Catalase/DS complex characterization. Characterization of catalase/DS complexes made at Cat:DS molar ratios of 1, 3, 5, 7, 10, and 13. (30A) Zeta-potential (mV) of complexes formed at different pHs, diluted 1000-fold in 10 mM NaCl. (30B) Zeta-potential of complexes formed at different pHs at original solution volume. (30C) Polydispersity (PDI) of complexes at different pHs. (30D) Size (nm) of complexes at different pHs.

FIG. 31. Lysozyme complexation with hydrophobic ion pairing agent, dextran sulfate. Hydrophobic ion complexes were made using lysozyme (MW 14 kDa) and dextran sulfate (DS) at varying molar ratios (DS/Lys Molar ratio (MR)=0.5, 1, 3, 5, 7, 9). Lysozyme only with no DS was used as a control. The state point diagram demonstrates the range of pH's at which insoluble precipitates form (● (black circle): precipitate with clear supernatant and higher volume than ▪ (black square): precipitate with clear solution; ▴ (black triangle): precipitate with cloudy solution) indicating formation of a successful hydrophobic ion complex. □ (white square): represents a turbid solution; ∘ (white circle): represents a translucent solution (no precipitate). Representative visuals of the various conditions can be found in FIG. 29B.

FIGS. 32A-32D. Lysozyme/DS complex characterization. Characterization of Lysozyme/DS complexes made at Lys:DS molar ratios of 0.5, 1, 3, 5, 7, 9. (32A) Zeta-potential (mV) of complexes formed at different pHs, diluted 1000-fold in 10 mM NaCl. (32B) Zeta-potential of complexes formed at different pHs at original solution volume. (32C) Polydispersity (PDI) of complexes at different pHs. (32D) Size (nm) of complexes at different pHs.

FIG. 33. Lysozyme/DS pH 7.46 activity. Preliminary data demonstrating lysozyme activity overtime at pH 7.46 once complexed with dextran sulfate.

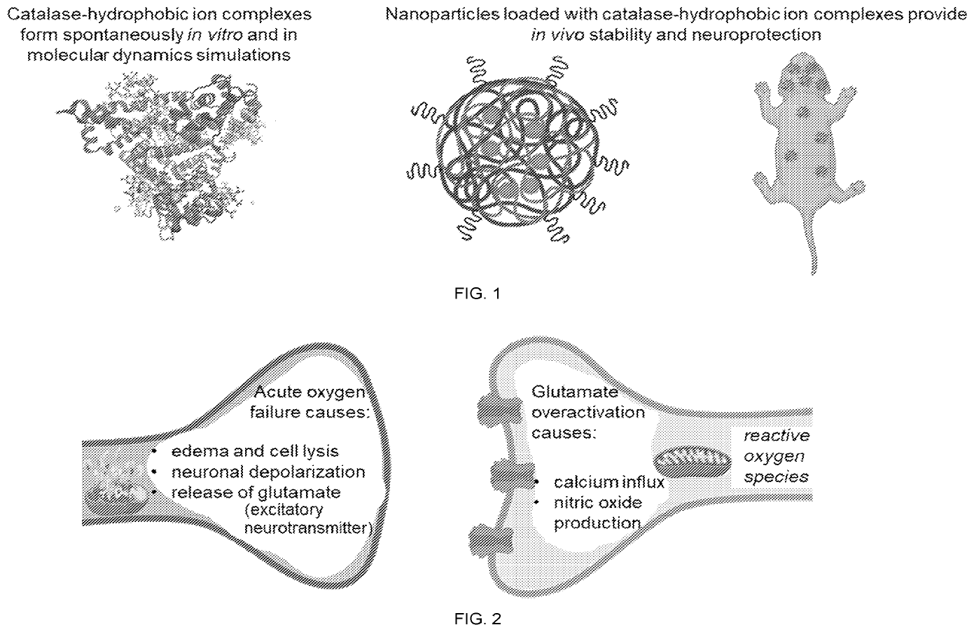

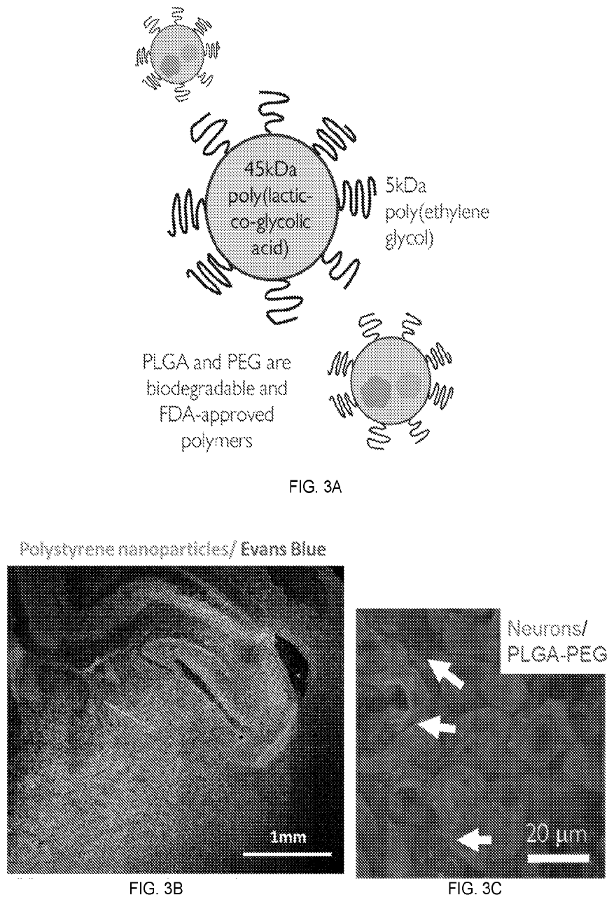

### DETAILED DESCRIPTION

Neonatal hypoxic-ischemic encephalopathy (HIE) is a devastating neurological condition that affects 1.3-4.7 in 1000 live births in the United States. Without treatment, ⅔ of affected infants with this condition will die or have a serious neurological disability, like cerebral palsy, seizures, intellectual disability, or developmental delays. The current standard-of-care, therapeutic hypothermia (TH), is only clinically implemented in cases of moderate or severe HIE and still leaves more than 30% of infants dead or with severe disability. One strategy to improve neonatal outcomes is to use a pharmaceutical agent to complement the neuroprotective mechanisms of TH. For example, catalase, a large (240 kDa) enzyme converts the reactive oxygen species (ROS) hydrogen peroxide to water and molecular oxygen and subsequently suppresses inflammation. For neurological applications, catalase can attenuate neuroinflammatory and apoptotic pathways in vitro, but its application in vivo has been limited due to its short half-life, proteolytic degradation, immunogenicity, and inability to penetrate the brain.

Nanoparticles serve as vehicles that can improve drug biodistribution and bioavailability. Drug-loaded biodegradable nanoparticles composed of poly(lactic-co-glycolic acid)-poly(ethylene glycol) (PLGA-PEG) have been shown to improve drug solubility, stability, circulation time, release kinetics, and transport to and within the brain parenchyma. Enzymes can particularly benefit from nanoparticle encapsulation as the polymer matrix provides protection from immune clearance and systemic degradation. However, the hydrophilic nature of enzymes limits their encapsulation into the hydrophobic core of PLGA-PEG nanoparticles.

The present disclosure describes enzyme-loaded polymeric nanoparticles wherein the lipophilicity of the enzyme is increased via hydrophobic ion pairing. In this technique, complexes are formed by electrostatic interactions between ionizable groups on the enzyme and an ion-pairing agent (hydrophobic ion). Because of the hydrophobicity of the resulting enzyme-hydrophobic ion complex (hydrophobic ion pairing complex), the enzyme can be loaded into a polymeric nanoparticle while retaining its activity.

In particular embodiments, an enzyme-loaded polymeric nanoparticle includes a hydrophobic ion pairing complex encapsulated within a polymer, wherein the hydrophobic ion pairing complex includes a hydrophobic ion and an enzyme. In particular embodiments, the polymer includes a hydrophobic polymer and polyethylene glycol (PEG). In particular embodiments, the hydrophobic polymer includes polylactic co-glycolic acid (PLGA), polylactic acid (PLA), polyglycolic acid (PGA), poly-ε-caprolactone (PCL), ethyl cellulose (EC), polybutylcyanoacrylate (PBCA), polypropylene oxide (PPO), or polyhydroxybutyrate (PHB). In particular embodiments, the hydrophobic polymer include PLGA. In particular embodiments, the hydrophobic ion includes dextran sulfate. In particular embodiments, the enzyme retains activity within the nanoparticle. In particular embodiments, the enzyme treats a neurological condition including an acute neurological injury or a chronic neurodegenerative condition. In particular embodiments, the enzyme treats a non-neurological condition including an oxidative stress burden, a lysosomal storage disease, or a metabolic deficiency. In particular embodiments, the enzyme is catalase or lysozyme.

The present disclosure includes an enzyme-loaded polymeric nanoparticle, methods of preparing a hydrophobic ion pairing complex, methods of preparing the enzyme-loaded polymeric nanoparticle, and methods of treating a condition with the enzyme-loaded polymeric nanoparticle.

Aspects of the current disclosure are now described with more detail and options as follows: (i) Enzymes; (ii) Hydrophobic Ion Pairing Complexes; (iii) Polymers; (iv) Surfactants; (v) Preparation of Nanoparticles; (vi) Compositions; (vii) Methods of Use; (viii) Kits; (ix) Exemplary Embodiments; (x) Experimental Examples; and (xi) Closing Paragraphs. These headings are provided for organizational purposes only and do not limit the scope or interpretation of the disclosure.

(i) Enzymes. The nanoparticles of the present disclosure include one or more enzymes. Herein, an enzyme refers to a molecule that catalyzes a chemical reaction involving a substrate.

In particular embodiments, the enzyme is used to treat a neurological condition.

Catalase, an antioxidant enzyme, is a promising therapeutic due to its ability to scavenge toxic reactive oxygen species and improve tissue oxygen status. Catalase is a large enzyme with a size of 240 kDa. In particular embodiments, the enzyme is catalase. In particular embodiments, catalase includes an amino acid sequence as set forth in NCBI Reference Sequence: NP_001743.1.

Lysozyme is a 14 kDa antimicrobial enzyme important in the innate immune system. It is often used for its analgesic effect and as a potentiating agent in antibiotic therapy. In particular embodiments, the enzyme is lysozyme. In particular embodiments, lysozyme includes an amino acid sequence as set forth in GenBank: AC037637.1.

In particular embodiments, the enzyme includes an anti-inflammatory enzyme, an excitotoxicity-altering enzyme, or an antioxidative enzyme.

In particular embodiments, the anti-inflammatory enzyme includes lysozyme, chymotrypsin, trypsin, serratiopeptidase, bromelain, ficin, papain, or serrapeptase. In particular embodiments, the excitotoxicity-altering enzyme includes glutamate decarboxylase, glutamate dehydrogenase, pyrroline-5-carboxylate synthase (P5CS), serum glutamate oxaloacetate transaminase (AST), or serum glutamate pyruvate transaminase (ALT). In particular embodiments, the antioxidative enzyme includes superoxide dismutase (SOD), glutamate cysteine ligase (GCL), or glutathione synthase (GS).

In particular embodiments, the enzyme is used to treat a non-neurological condition. In particular embodiments, a non-neurological condition includes oxidative stress burden, lysosomal storage disease, or a metabolic enzyme deficiency.

In particular embodiments, an enzyme used to treat an oxidative stress burden includes superoxide dismutase (SOD), catalase (CAT), glutathione peroxidase (GPx), aldehyde oxidase, glyoxalase, myeloperoxidase, nitric oxide synthase, and/or sphingomyelin phosphodiesterase 3.

In particular embodiments, an enzyme used to treat a lysosomal storage disease includes glucocerebrosidase, sphingomyelin cholinephosphohydrolase, acid sphingomyelinase, acid alpha-glucosidase, aspartylglucosaminidase, alpha-galactosidase A, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, and/or acid lipase.

In particular embodiments, an enzyme used to treat a metabolic enzyme deficiency includes glucose-6-phosphatase, fructose-1,6-bisphosphatase, tibose-phosphate isomerase, transaldolase, succinate dehydrogenase, glucose-6-phosphate dehydrogenase, fumarase, pyruvate dehydrogenase complex, and/or N-acetylglutamate synthetase.

In particular embodiments, enzyme-loaded nanoparticles retain enzymatic activity following administration to a subject at least until a therapeutic and/or physiological objective is achieved. In particular embodiments, enzyme-loaded nanoparticles retain enzymatic activity in degradative environments. In particular embodiments, enzyme-loaded nanoparticles retain enzymatic activity in serum or serum-like conditions.

Enzyme activity can be measured in vitro, ex vivo, or in vivo. Enzyme activity can be measured using spectrophotometric assays, magnetic resonance spectroscopy (MRS), magnetic resonance imaging (MRI), Förster resonance energy transfer (FRET) analysis, mass spectrometry, microdialysis, activity-based probes, and/or electroosmotic sampling. Enzyme activity can be reported as active units (AU) per mass. The mass of the enzyme can be determined using protein quantification assays known in the art including UV absorption, biuret methods (e.g., BCA assays and Lowry assays), colorimetric dye-based methods (e.g. Bradford assay), and/or fluorescent dye methods (e.g. EZQ fluorescent assay and Qubit protein assay).

In particular embodiments, the enzyme retains 60%, 65%, 70%, 75%, 80%, 85%, 90%, 95%, or 100% enzyme activity compared to a benchmark enzyme activity. In particular embodiments, the benchmark enzyme activity is determined by the AU per mass according to the enzyme's manufacturer. In particular embodiments, the benchmark enzyme activity is determined by the AU per mass before administration to a subject. In particular embodiments, the benchmark enzyme activity is determined by the AU per mass at the time the enzyme is introduced to degradative conditions. In particular embodiments, the benchmark enzyme activity is determined by the AU per mass after encapsulation into nanoparticle. In particular embodiments, the benchmark enzyme activity is determined by the AU per mass of an enzyme not in degradative conditions. In particular embodiments, the benchmark enzyme activity of catalase is 2000-5000 AU/mg.

Enzyme activity is affected by several factors including strength of degradative conditions, pH, temperature, and time in degradative conditions. Degradative conditions can be physical or chemical. Physical degradation can be due to interfacial adsorption and aggregation which can lead to unfolding of the protein. Physical degradation can also be due to agitation, temperature, or pH stresses. Chemical degradation includes oxidation, isomerization, hydrolysis, disulfide scrambling, beta elimination, deamidation, and adduct formation. The principal hydrolytic mechanisms of degradation include peptide bond hydrolysis, deamidation of asparagine and glutamine and the isomerization of aspartic acid. In particular embodiments, degradative conditions include native conditions in the body. In particular embodiments, degradative conditions include the presence of serum, pronases, proteases, or pH stress.

(ii) Hydrophobic Ion Pairing Complexes. Hydrophobic ion pairing (HIP) is an interaction between a pair of oppositely charged molecules or reagents held together by Coulomb attraction. In particular embodiments, HIP, as used herein, refers to the interaction between an enzyme and a hydrophobic ion to form an HIP complex based on Coulomb attraction. HIP complex or hydrophobic ion pair, as used herein, means a complex formed by the enzyme and the hydrophobic ion. In particular embodiments, as used herein, a hydrophobic ion is a charged molecule, such as a charged element, a charged small molecule, or a charged protein. In particular embodiments, the hydrophobic ion includes a hydrophobic acid or salt of a hydrophobic acid. In particular embodiments, the hydrophobic ion includes bile acids or salts, fatty acids or salts, lipids, or amino acids. In some embodiments, the hydrophobic ion is negatively charged (anionic). Examples of negatively charged hydrophobic ions include: dextran sulfate, sodium dodecyl sulfate (SDS), sodium sulfosuccinate (AOT), sodium oleate (OA), human serum albumin (HSA), sodium deoxy cholate, sodium cholate, anionic lipids, amino acids, or any combination thereof. In particular embodiments, the hydrophobic acid can be a carboxylic acid (such as monocarboxylic acid, dicarboxylic acid, tricarboxylic acid), sulfinic acid, sulfenic acid, or sulfonic acid. In particular embodiments, the hydrophobic ion includes a bile acid such as taurocholic acid (TA).

In particular embodiments, HIP may increase the hydrophobicity and/or lipophilicity of the enzyme. In particular embodiments, increasing the hydrophobicity and/or lipophilicity of the enzyme may be beneficial for nanoparticle formulation and may provide higher solubility of the enzyme in organic solvents. Without being limited by theory, it is believed that nanoparticle formulations comprising HIP pairs have improved formulation properties such as drug load and/or release profile. In particular embodiments, slow release of the enzyme from the nanoparticles can occur by a decrease in the solubility of the enzyme in aqueous solution. Further, the complexation of the enzyme with a large hydrophobic ion may slow diffusion of the enzyme within the polymeric matrix. In particular embodiments, the HIP occurs without covalent conjugation of the hydrophobic ion to the enzyme.

The strength of the HIP can affect the drug loading and release rate of the nanoparticles. In particular embodiments, the strength of HIP can be increased by increasing the magnitude of the difference between the pKa of the enzyme and the pKa of the hydrophobic ion. In particular embodiments, the ion pairing conditions may affect the drug loading and release rate of the nanoparticles.

Examples of dissociation constants (pKa) and log P values in hydrophobic acids, saturated fatty acids, unsaturated fatty acids, aromatic acids, bile acids, polyelectrolytes, water are described in WO2014/043,625. The strength of hydrophobic acids, the difference between the pKa of hydrophobic acids and the pKa of the enzyme, the log P of hydrophobic acids, the phase transition temperature of hydrophobic acids, the molar ratio of hydrophobic acids to the enzyme, and the concentration of hydrophobic acids are also disclosed in WO2014/043,625.

In particular embodiments, preparation of nanoparticles using an HIP complex increase drug load by 50%, 100%, 2 times, 3 times, 4 times, 5 times, 6 times, 7 times, 8 times, 9 times, 10 times, or more than 10 times.

In particular embodiments, the hydrophobic ion to enzyme charge ration is 1:1, 1:2, 1:3, 1:4, 1:5, 1:6, 1:7, 1:8, 1:9, 1:10, 2:1, 3:1, 4:1, 5:1, 6:1, 7:1, 8:1, or 9:1. In particular embodiments, the hydrophobic ion to enzyme charge ratio is 1:1.

In particular embodiments, a method of preparing an HIP complex includes mixing, within a solution, the hydrophobic ion and an enzyme to form an HIP complex within the solution. In particular embodiments, the HIP complex within the solution is removed from solution by centrifugation. In particular embodiments, the enzyme retains activity within the HIP complex. In particular embodiments, the HIP complex is lyophilized. In particular embodiments, the HIP complex is dissolved in a water miscible organic solvent.

(iii) Polymers. In particular embodiments, the HIP complex is encapsulated in a polymeric nanoparticle to form an enzyme-loaded polymeric nanoparticle.

Any suitable polymer can be used in the disclosed nanoparticles. Polymers can be natural or unnatural (synthetic) polymers. Polymers can be homopolymers or copolymers including two or more monomers. In terms of sequence, copolymers can be random, block, or include a combination of random and block sequences. Typically, polymers are organic polymers.

The term “polymer,” as used herein, is given its ordinary meaning as used in the art, i.e., a molecular structure including one or more repeat units (monomers), connected by covalent bonds. The repeat units may all be identical, or in some cases, there may be more than one type of repeat unit present within the polymer. In some cases, the polymer can be biologically derived, i.e., a biopolymer. Examples of polymers include peptides or proteins. In some cases, additional moieties may also be present in the polymer, for example, biological moieties such as those described below. If more than one type of repeat unit is present within the polymer, then the polymer is said to be a “copolymer.” It is to be understood that in any embodiment employing a polymer, the polymer being employed may be a copolymer in some cases. In particular embodiments, a polymer is a copolymer. The repeat units forming the copolymer may be arranged in any fashion. For example, the repeat units may be arranged in a random order, in an alternating order, or as a block copolymer, i.e., including one or more regions each including a first repeat unit (e.g., a first block), and one or more regions each including a second repeat unit (e.g., a second block), etc. Block copolymers may have two (a diblock copolymer), three (a triblock copolymer), or more numbers of distinct blocks.

In particular embodiments, disclosed nanoparticles include copolymers, which, in some embodiments, describe two or more polymers (such as those described herein) that have been associated with each other, usually by covalent bonding of the two or more polymers together. Thus, a copolymer may include a first polymer and a second polymer, which have been conjugated together to form a copolymer.

In particular embodiments, a polymer can be hydrophobic, hydrophilic, or amphiphilic. A hydrophobic (water-resistant) polymer is a material that generally repels water while a hydrophilic polymer is a material that generally attracts water. An amphiphilic polymer has a hydrophilic portion and a hydrophobic portion, or a relatively hydrophilic portion and a relatively hydrophobic portion. A hydrophilic or a hydrophobic polymer can be identified, for example, by preparing a sample of the polymer and measuring its contact angle with water (typically, the polymer will have a contact angle of less than 60°, while a hydrophobic polymer will have a contact angle of greater than 60°). In some cases, the hydrophilicity of two or more polymers may be measured relative to each other, i.e., a first polymer may be more hydrophilic than a second polymer. For instance, the first polymer may have a smaller contact angle than the second polymer.

Examples of hydrophobic polymers include polyhydroxy acids including polylactic co-glycolic acid (PLGA), polylactic acid (PLA), and polyglycolic acid; polyhydroxyalkanoates such as polyhydroxybutyrate (PHB); poly-ε-caprolactone (PCL); ethyl cellulose (EC); polycyanoacrylate such as polybutylcyanoacrylate (PBCA); polypropylene oxide (PPO); polyactylonitrile; poly(vinylidene fluoride); polyvinylpyrrolidone; polytetrafluoroethylene; polyvinylpyrrolidone; polypropylene glycol; polystyrene; polyethylene; nylon; poly(propylene carbonate); poly(ethylene terephthalate); poly (orthoesters); polyanhydrides; poly (phosphazene); poly (lactide-co-caprolactone); polycarbonates such as tyrosine polycarbonates; polyamides (including synthetic and natural polyamides), polypeptides, and poly (amino acids); polyesteramides; polyester; poly (dioxanone); poly (alkylene alkylates); hydrophobic polyethers; polyurethane; polyether esters; polyacetals; polyacrylates; polymethyl methacrylate; polysiloxanes; poly (oxyethylene)/poly (oxypropylene) copolymers; polyketal; polyphosphate; polyhydroxy valerate; polyalkylene oxalate; polyalkylene succinates; poly (maleic acid); and/or copolymers or derivative thereof. In particular embodiments, the hydrophobic polymer is PLGA.

Example of hydrophilic polymers include cellulose polymers such as starch and polysaccharides; polyalkylene glycols and polyalkylene oxides such as polyethylene glycol (PEG), polypropylene glycol (PPG), and poly (ethylene oxide) (PEO); poly (oxyethylated polyols); hydrophilic polypeptides; poly (amino acids) such as poly-L-glutamic acid (PGS), gamma-polyglutamic acid, poly-L-aspartic acid, poly-L-serine, or poly-L-lysine; poly (olefinic alcohol); polyvinylpyrrolidone; poly (hydroxyalkylmethacrylamide); poly (hydroxyalkyl methacrylate); poly (saccharides); poly (hydroxy acid); poly (vinyl alcohol) (PVA); polyoxazoline; and/or copolymers or derivatives thereof. A polymer (e.g., copolymer) containing PEG repeat units can also be referred to as a “PEGylated” polymer. In particular embodiments, the hydrophilic polymer is PEG.

Amphiphilic polymers can be polymers containing hydrophobic polymer blocks and hydrophilic polymer blocks. The hydrophobic polymer block may contain one or more of the above hydrophobic polymers or derivatives or copolymers thereof. The hydrophilic polymer block may contain one or more of the above hydrophilic polymers or derivatives or copolymers thereof. In some embodiments the amphiphilic polymer is a di-block polymer containing hydrophobic ends formed from hydrophobic polymers and hydrophilic ends formed from hydrophilic polymers. The nanoparticles may contain two or more amphipathic polymers.

In particular embodiments, the HIP complex is encapsulated in a polymeric nanoparticle. In particular embodiments, the polymer is a hydrophobic polymer. In particular embodiments, the hydrophobic polymer is PLGA. In particular embodiments, the polymer is a copolymer. In particular embodiments, the copolymer includes a hydrophobic polymer and PEG. In particular embodiments, the copolymer includes PLGA and PEG. In particular embodiments, the copolymer includes PLGA (45k) and PEG (5k).

In particular embodiments, a polymer is biocompatible. A biocompatible polymer does not typically induce an adverse response when inserted or injected into a living subject, for example, without significant inflammation and/or acute rejection of the polymer by the immune system, for instance, via a T-cell response.

One simple test to determine biocompatibility can be to expose a polymer to cells in vitro; biocompatible polymers are polymers that typically will not result in significant cell death at moderate concentrations, e.g., at concentrations of 50 μg/10⁶ cells. For instance, a biocompatible polymer may cause less than 20% cell death when exposed to cells, even if phagocytosed or otherwise taken up by such cells. Examples of biocompatible polymers that may be useful in various embodiments include polydioxanone (PDO), polyhydroxyalkanoate, polyhydroxybutyrate, poly(glycerol sebacate), polyglycolide (i.e., poly(glycolic) acid) (PGA), polylactide (i.e., poly(lactic) acid) (PLA), poly(lactic) acid-co-poly(glycolic) acid (PLGA), polycaprolactone, or copolymers or derivatives thereof.

In particular embodiments, biocompatible polymers may be biodegradable, i.e., the polymer is able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In particular embodiments, the biodegradable polymer and their degradation byproducts can be biocompatible.

Biodegradable polymers can include: polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidones, polyglycolides, polysiloxanes, polyurethanes and copolymers thereof, alkyl celluloses such as methyl cellulose and ethyl cellulose, hydroxyalkyl celluloses such as hydroxypropyl cellulose, hydroxy-propyl methyl cellulose, and hydroxybutyl methyl cellulose, cellulose ether, cellulose ester, nitro cellulose, cellulose acetate, cellulose propionate, cellulose acetate butyrate, cellulose acetate phthalate, carboxyethyl cellulose, cellulose triacetate, cellulose sulfate sodium salt, polymers of acrylic and methacrylic esters such as poly (methyl methacrylate), poly (ethyl methacrylate), poly (butyl methacrylate), poly (isobutyl methacrylate), poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), polyethylene, polypropylene poly (ethylene glycol), poly (ethylene oxide), poly (ethylene terephthalate), poly (vinyl alcohol), poly (vinyl acetate, poly vinyl chloride polystyrene and polyvinylpyrrolidone, its derivatives, its linear and branched copolymers and block copolymers, and its exemplary biodegradable polymers include: polyester, poly (orthoester), poly (ethylene imine), poly (caprolactone), poly (hydroxyalkanoate), poly (hydroxyvalrate), polyanhydrides, poly (acrylic acid), polyglycolides, poly (urethanes), polycarbonates, polyphosphate esters, polyphosphazenes, derivatives thereof, linear and branched copolymers and block copolymers thereof, and their formulation. In some embodiments the nanoparticles contain biodegradable polyesters or polyanhydrides such as poly (lactic acid), poly (glycolic acid), and poly (lactic acid-co-glycolic acid).

In particular embodiments, the molecular weight (or the ratio of molecular weights of a copolymer) of the polymers can be optimized for effective treatment as disclosed herein. For example, the molecular weight of a polymer may influence nanoparticle degradation rate, solubility, water uptake, and drug release kinetics. For example, the molecular weight of the polymer (or the ratio of molecular weights of a copolymer) can be adjusted such that the nanoparticle biodegrades in the subject being treated within a reasonable period of time (ranging from a few hours to 1-2 weeks, 3-4 weeks, 5-6 weeks, 7-8 weeks, etc.).

In particular embodiments, a polymer can include a number average molecular weight ranging from 1 kDa to 6 kDa, from 4 kDa to 6 kDa, from 4 kDa to 10 kDa, from 6 kDa to 10 kDa, from 2 kDa to 10 kDa, from 15 kDa to 20 kDa, from 10 kDa to 25 kDa, from 15 kDa to 30 kDa, from 25 kDa to 50 kDa, or from 30 kDa to 60 kDa. In particular embodiments, a copolymer including a first polymer and a second polymer can include a number average molecular weight of the first polymer ranging from 1 kDa to 6 kDa, from 4 kDa to 6 kDa, from 4 kDa to 10 kDa, from 6 kDa to 10 kDa, from 2 kDa to 10 kDa, from 15 kDa to 20 kDa, from 10 kDa to 25 kDa, from 15 kDa to 30 kDa, from 25 kDa to 50 kDa, from 30 kDa to 60 kDa, from 60 kDa to 150 kDa, from 150 kDa to 300 kDa, or from 300 kDa to 500 kDa, and a number average molecular weight of the second polymer ranging from 1 kDa to 6 kDa, from 4 kDa to 6 kDa, from 4 kDa to 10 kDa, from 6 kDa to 10 kDa, from 2 kDa to 10 kDa, from 10 kDa to 20 kDa, from 20 kDa to 30 kDa, or from 30 kDa to 40 kDa. In particular embodiments, a copolymer including PLGA and PEG can include a number average molecular weight of the PLGA ranging from 1 kDa to 6 kDa, from 4 kDa to 6 kDa, from 4 kDa to 10 kDa, from 6 kDa to 10 kDa, from 2 kDa to 10 kDa, from 15 kDa to 20 kDa, from 10 kDa to 25 kDa, from 15 kDa to 30 kDa, from 25 kDa to 50 kDa, from 30 kDa to 60 kDa, from 60 kDa to 150 kDa, or from 150 kDa to 300 kDa, and a number average molecular weight of the PEG ranging from 1 kDa to 6 kDa, from 4 kDa to 6 kDa, from 4 kDa to 10 kDa, from 6 kDa to 10 kDa, or from 2 kDa to 10 kDa.

In particular embodiments, a copolymer including a first polymer and a second polymer includes a weight ratio of first polymer to second polymer of 1:0.1, 1:0.5, 1:1, 1:1.5, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In particular embodiments, a copolymer including a first polymer, a second polymer, and a third polymer includes a weight ratio of first polymer to second polymer to third polymer of 1:0.1:1, 1:0.1:2, 1:0.1:3, 1:0.1:4, 1:0.1:5, 1:0.1:6, 1:0.1:7, 1:0.1:8, 1:0.1:9, 1:0.1:10, 1:0.1:12, 1:0.1:14, 1:0.1:16, 1:0.1:18, 1:0.1:20, 1:0.5:1, 1:0.5:2, 1:0.5:3, 1:0.5:4, 1:0.5:5, 1:0.5:6, 1:0.5:7, 1:0.5:8, 1:0.5:9, 1:0.5:10, 1:0.5:12, 1:0.5:15, 1:0.5:17, 1:0.5:19, 1:0.5:20, 1:1:1, 1:1:2, 1:1:3, 1:1:4, 1:1:5, 1:1:6, 1:1:7, 1:1:8, 1:1:9, 1:1:10, 1:1:12, 1:1:14, 1:1:16, 1:1:18, 1:1:20, 1:1.5:5, 1:2:7, 1:3:4, 1:4:1, 1:5:1, 1:6:1, 1:7:0.5, 1:8:3, 1:9:0.7, 1:10:20, 1:11:15, 1:12:4, 1:13:13, 1:14:17, 1:15:2, 1:16:4, 1:17:17, 1:18:1, 1:19:20, or 1:20:10.

In particular embodiments, a nanoparticle includes from 1 to 99 weight percent of a polymer (or copolymer), from 10 to 99 weight percent, from 50 to 99.75 weight percent, from 20 to 80 weight percent, from 40 to 80 weight percent, or from 30 to 50 weight percent, or from 70 to 90 weight percent, from 70 to 99.75 weight percent, from 80 to 99.75 weight percent, from 70 to 80 weight percent, or from 85 to 95 weight percent of a polymer. In particular embodiments, a nanoparticle includes 50 weight percent, 55 weight percent, 60 weight percent, 65 weight percent, 70 weight percent, 75 weight percent, 80 weight percent, 85 weight percent, 90 weight percent or 95 weight percent polymer.

(iv) Surfactants. Surfactants can be classified as nonionic, anionic, cationic, or amphoteric depending on the charge of the hydrophilic head. Nonionic surfactants are neutral and do not have any charge on their hydrophilic end. Anionic surfactants have a negative charge while cationic surfactants have a positive charge on their hydrophilic end. Amphoteric surfactants, also referred to as zwitterionic surfactants, can have either a positive, negative, or neutral net charge on their hydrophilic end depending on the pH of the environment. In acidic environments, the amphoteric surfactant behaves like a cationic surfactant and in alkaline environments, it behaves similar to an anionic surfactant. Particular embodiments utilize surfactants as an emulsifying agent. An emulsifying agent is a surfactant that stabilizes emulsions by coating nanoparticles or droplets within an emulsion, preventing the nanoparticles or droplets from aggregating. In particular embodiments, surfactants coating the nanoparticle enhance nanoparticle stability, alter diffusivity within the body, and determine degree of cellular uptake.

Nonionic surfactants include sorbitan esters, polyethylene glycol sorbitan fatty acid esters, polyoxyethylene alkyl ethers, polyoxyethylene fatty acid esters, polyethylene-polypropylene glycols, saturated polyglycolized glycerides, polyethylene glycols, quillaia, polyethylene glycol stearates, and polyethylene glycol glycerides.

Sorbitan esters include sorbitan monolaurate, sorbitan monopalmitate, sorbitan monostearate, sorbitan tristearate, sorbitan monooleate, and sorbitan trioleate.

Polyethylene glycol sorbitan fatty acid esters include polyethylene glycol sorbitan monolaurate (polysorbate 20), polyethylene glycol sorbitan monopalmitate (polysorbate 40), polyethylene glycol sorbitan monostearate (polysorbate 60), polyethylene glycol sorbitan tristearate (polysorbate 65), polyethylene glycol sorbitan monooleate (polysorbate 80), polyethylene glycol sorbitan trioleate (polysorbate 85), polyethylene glycol sorbitan hexaoleate, and polyethylene glycol sorbitan tetraoleate.

Polyoxyethylene fatty acid esters include polyoxyl 40 hydrogenated castor oil.

Polyethylene-polypropylene glycols include polyethylene-polypropylene glycol (poloxamer) 124, poloxamer 188, poloxamer 407, poloxamer 108, poloxamer 217, poloxamer 238, poloxamer 288, poloxamer 338, poloxamer 182, poloxamer 183, poloxamer 212, poloxamer 331, or poloxamer 335.

Saturated polyglycolized glycerides include lauroyl macrogol 32 glycerides.

Polyethylene glycols include polyethylene glycol (PEG). In particular embodiments, the molecular weight of PEG ranges from 1,000 Da to 30,000 Da.

Quillaia include quillaia/quillaja saponins or quillaia extract.

Polyethylene glycol stearates include PEG 8 stearates or PEG 40 stearates.

Polyethylene glycol hydrogenated castor oils include PEG 25 hydrogenated castor oil and PEG 40 hydrogenated castor oil.

Polyethylene glycol glycerides include PEG 8 caprylic/capric glycerides or PEG 300 oleic glycerides.

Common anionic surfactants include dioctyl sodium sulfosuccinate (DOSS), perfluorooctanesulfonate (PFOS), linear alkylbenzene sulfonates, sodium lauryl ether sulfate, lignosulfonate, and sodium stearate.

Cationic surfactants include benzalkonium chloride (BAC), cetylpyridinium chloride (CPC), Benzethonium chloride (BZT), cetyl trimethylammonium bromide (CTAB), and cetyl trimethylammonium chloride (CTAC).

Amphoteric surfactants include lauryl betain, betaine citrate, sodium lauroamphoacetate, sodium hydroxymethylglycinate, (carboxymethyl)dimethyl-3-[(1-oxododecyl)amino] propyl ammonium hydroxide, rennin, coco alkyldimethyl betaines, (carboxymethyl) dimethyloleylammonium hydroxide, cocoamidopropyl betaine, and (carboxylatomethyl) dimethyl (octadecyl)ammonium.

In particular embodiments, the surfactant is a polysorbate or a poloxamer. In particular embodiments, the polysorbate is polysorbate 80 (P80). In particular embodiments, the poloxamer includes poloxamer 188 (F68) or poloxamer 407 (F127). In particular embodiments, the surfactant is a nonionic surfactant or anionic surfactant. In particular embodiments, the nonionic surfactant is polyvinyl alcohol. IN particular embodiments, the anionic surfactant is cholic acid. In particular embodiments, the surfactant includes two or more surfactants

Surfactants can also be characterized by the hydrophilic-lipophilic balance (HLB) which is the balance of the size and strength of the hydrophilic and lipophilic moieties of a surfactant molecule ranging from 0 to 20. Lower values are more hydrophobic and higher numbers are more hydrophilic. In particular embodiments anti-foaming agents have an HLB of 1.5-3, water-in-oil emulsifiers have an HLB of 3-6, wetting agents have an HLB of 7-9, oil-in-water emulsifiers have an HLB of 8-12, and solubilizers have an HLB of 15-20. In particular embodiments, the surfactant has an HLB value greater than 4, 5, 6, 7, 8, 9, 10, 11, 12, 13, 14, 15, 16, 17, 18, 19, or 20. In other embodiments, the surfactant has an HLB value between 4-6, 5-9, 8-17, 9-16.7, 9-16, 9-15, 9-14, 10-17, 10-16.7, 10-16, 10-15, 14-16, 14-17, 15-17, and between 10-14. In certain examples, the surfactant or plurality of surfactants can be chosen according to the HLB requirement of the nanoparticle.

In particular embodiments, the nanoparticles are coated in surfactant in an amount selected from: less than 20% w/w, less than 21% w/w, less than 22% w/w, less than 23% w/w, less than 24% w/w, less than 25% w/w, less than 26% w/w, less than 27% w/w, less than 28% w/w, less than 29% w/w, less than 30% w/w, less than 31% w/w, less than 32% w/w, less than 33% w/w, less than 34% w/w, and less than 35% w/w.

The surfactant choice can affect the accumulation of nanoparticles in different areas of the body, diffusivity, and cellular uptake. In particular embodiments, the choice of surfactant can influence the accumulation of the nanoparticle in the brain, liver, spleen, kidney, heart, or lungs. In particular embodiments, the nonionic surfactants increase blood brain barrier penetration by the nanoparticles. In particular embodiments, the nonionic surfactants increase nanoparticle uptake in the brain parenchyma. In particular embodiments, charged surfactants enable faster overall nanoparticle diffusion within the brain. In particular embodiments, polysorbates increase nanoparticle uptake in the brain parenchyma. In particular embodiments, poloxamers increase nanoparticle distribution throughout whole brain. In particular embodiments, the nanoparticle does not require a targeting ligand in order to target biological structures. Herein, a targeting ligand refers to a molecule or protein attached to the surface of a nanoparticle which enables the recognition of specific antigens or receptors on target cells. In particular embodiments, the surfactant is responsible for the transportation of the nanoparticle through the blood brain barrier.

(v) Preparation of Nanoparticles. The present disclosure includes systems and methods of making disclosed nanoparticles. In particular embodiments, a method of making a nanoparticle includes preparing an HIP complex from an enzyme and hydrophobic ion; providing a polymer (or copolymer); combining the HIP complex and the polymer to form a nanoparticle; and recovering the nanoparticles. In particular embodiments, the combining includes mixing the HIP complex and polymer within a solvent. In particular embodiments, the combining can include a nanoprecipitation method. In particular embodiments, the combining can include a double emulsion method.

In particular embodiments, the HIP complex is lyophilized. In particular embodiments, the HIP complex is dissolved in a water miscible organic solvent.

In particular embodiments, the polymer is dissolved in a water immiscible organic solvent. In particular embodiments, the polymer is dissolved in a water miscible organic solvent.

Examples of water immiscible organic solvent include chloroform, dichloromethane, and acyl acetate. In particular embodiments, the water immiscible organic solvent includes dichloromethane

Examples of water miscible organic solvent include acetone, acetaldehyde, acetic acid, acetonitrile, 1,2-Butanediol, 1,3-Butanediol, 1,4-Butanediol, 2-Butoxyethanol, butyric acid, diethanolamine, diethylenetriamine, dimethylformamide, dimethoxyethane, dimethyl sulfoxide, 1,4-Dioxane, ethanol, ethylamine, ethylene glycol, formic acid, furfuryl alcohol, glycerol, methanol, methyl diethanolamine, methyl isocyanide, N-Methyl-2-pyrrolidone, 1-Propanol, 1,3-Propanediol, 1,5-Pentanediol, 2-Propanol, propanoic acid, propylene glycol, pyridine, tetrahydrofuran, and triethylene glycol.

An aqueous solution is a solution in which the solvent is water. In particular embodiments, the aqueous solution includes surfactant dissolved in water. Surfactants may, in some instances, act as emulsifiers or stabilizers for the compositions disclosed herein. Suitable surfactants may be cationic surfactants, anionic surfactants, or nonionic surfactants. Examples of surfactants are described elsewhere herein. In particular embodiments, the surfactant is polysorbate 80 (P80). In particular embodiments, the surfactant is poloxamer 188 (F68) or poloxamer 407 (F127).

In particular embodiments, a nanoprecipitation process includes mixing the polymer and HIP complex in a water miscible organic solvent. The organic solvent is then removed suddenly when it is added to a much larger volume of an aqueous solution. The HIP complexes are encapsulated in the sparingly soluble polymer during precipitation. In particular embodiments, lyophilized HIP complexes are dissolved in a water miscible organic solvent. In particular embodiments, the water miscible organic solvent is DMSO. In particular embodiments, the polymer (e.g., PLGA-PEG) is dissolved in a water miscible organic solvent. In particular embodiments, the water miscible organic solvent is acetone. In particular embodiments, the HIP complexes in solvent is mixed with the polymer in solvent to form a mixture. In particular embodiments, the mixing includes vortexing. In particular embodiments, the mixture is added dropwise into an aqueous solution. In particular embodiments, aqueous solution includes 1% P80 in water.

In particular embodiments, a double emulsion process includes a solid in oil in water (S/O/W) double emulsion process. In a S/O/W double emulsion process, the solid, lyophilized HIP complex is combined with the polymer in water immiscible organic solvent to form the solid in oil emulsion. The homogenized solid in oil emulsion is then added to an aqueous phase to form a second emulsion, or solid in oil in water emulsion, to form nanoparticles. In particular embodiments, the formation of a double emulsion is achieved using a homogenizer or coarse mixing followed by homogenization. The solvent is then removed to leave discrete polymeric nanoparticles. In particular embodiments, the water immiscible organic solvent is dichloromethane.

In particular embodiments, a double emulsion process includes a water in oil in water (W/O/W) double emulsion process. In a W/O/W double emulsion process, the HIP complex is in an aqueous solution and combined with the polymer in water immiscible organic solvent to form the water in oil emulsion. The homogenized water in oil emulsion is then added to an aqueous phase to form a second emulsion, or water in oil in water emulsion, to form nanoparticles. In particular embodiments, the formation of a double emulsion is achieved using a homogenizer or coarse mixing followed by homogenization. The solvent is then removed to leave discrete polymeric nanoparticles. During this process, the HIP complex is retained within aqueous droplets that fill pores in the polymeric matrix.

A double emulsion process includes a primary emulsion which can be prepared and then emulsified to form a fine emulsion. Primary emulsions may be formed, for example, using simple mixing and high-pressure homogenizers, probe sonicators, stir bars, or rotor stator homogenizers. The primary emulsion can be formed into a fine emulsion, for example through a homogenizer, through the use of a probe sonicator or a high-pressure homogenizer. For example, when a high-pressure homogenizer is used, the pressure used may be 4000 to 8000 psi, or 4000 to 5000 psi. In particular embodiments, emulsion includes sonication at 5-60% amplitude. In particular embodiments, emulsion includes sonication at 20% amplitude. In particular embodiments, emulsion includes stirring at 50-15,000 rpm. In particular embodiments, emulsion includes stirring at 500 rpm.

In particular embodiments, recovering the nanoparticles uses centrifugation, solvent evaporation and/or filtration. In particular embodiments, recovering the nanoparticles further uses washing steps.

Centrifugation is a technique used for separation of nanoparticles from a solution according size, shape, density, and/or viscosity. Ultracentrifugation is a type of centrifugation at a high speed. The rotation speed and centrifugation time will depend on the nanoparticles and solution to be separated. In particular embodiments, the centrifuge speed is 1,000 rpm to 150,000 rpm. In particular embodiments, the centrifuge speed is 1,000 rpm, 5,000 rpm, 7,000 rpm, 10,000 rpm, 12,000 rpm, 15,000 rpm, 17,000 rpm, 20,000 rpm, 50,000 rpm, 70,000 rpm, 100,000 rpm, 125,000 rpm, or 150,000 rpm. In particular embodiments, the centrifugal force is 500×g to 300,000×g. In particular embodiments, the centrifugal force is 500×g, 600×g, 1,000×g, 2,000×g, 5,000×g, 5,400×g, 10,000×g, 15,000×g, 30,000×g, 50,000×g, 75,000×g, 100,000×g, 150,000×g, 200,000×g, 250,000×g, or 300,000×g. In particular embodiments, the nanoparticles and solution are centrifuged for 1 minute, 2 minutes, 3 minutes, 5 minutes, 7 minutes, 10 minutes, 12 minutes, 15 minutes, 30 minutes, 1 hour, 2 hours, or 3 hours. The temperature during centrifugation can be controlled. In particular embodiments, the nanoparticles and solution are centrifuged at 0° C., 1° C., 4° C., 5° C., 6° C., 7° C., 8° C., 9° C., 10° C., 12° C., 13° C., 14° C., 15° C., 17° C., 18° C., 19° C., 20° C., 21° C., 22° C., 23° C., 24° C., 25° C., 26° C., 27° C., 28° C., 29° C., 30° C., 31° C., 32° C., 33° C., 35° C., 36° C., 37° C., 38° C., 39° C., 40° C., 41° C., 42° C., or 43° C.

Solvent evaporation or dilution may be required for complete solvent extraction and solidification of the nanoparticles. For better control over the kinetics of the extraction and for a more scalable process, solvent dilution via aqueous quenching can be used. For example, the emulsion can be diluted with cold water to a concentration sufficient to dissolve all organic solvents to form a quenched phase. Quenching may be performed at least partially at a temperature of 5° C. or less. For example, the water used in the quench may be at a temperature below room temperature (e.g., 0 to 10° C., or 0 to 5° C.).

In various embodiments, the nanoparticles are recovered by filtration. For example, ultrafiltration membranes can be used. Exemplary filtration can be performed using a tangential flow filtration system. For example, the nanoparticles can be selectively separated by using a membrane having a pore size suitable for holding the nanoparticles while passing through the solutes, colloids, and organic solvents. Exemplary membranes with a molecular weight cut-off of 300-500 kDa (5-25 nm) can be used.

(vi) Compositions. Nanoparticles can be formulated alone or in combination into compositions for administration to subjects in one or more pharmaceutically acceptable carriers.

Exemplary generally used pharmaceutically acceptable carriers include any and all absorption delaying agents, antioxidants (e.g., ascorbic acid, methionine, vitamin E), binders, buffering agents, bulking agents or fillers, chelating agents (e.g., EDTA), coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts. In particular embodiments, the choice of buffering agent depends on the pH range of the buffering agent. In particular embodiments, the buffering agent must have a pH range that overlaps with the pH range required to form HIP complexes.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the nanoparticles. Typical stabilizers can include PEG; polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

In particular embodiments, the nanoparticles can be lyophilized to increase their shelf life. In some embodiments, the composition also includes a lyophilizer. In particular embodiments, the lyophilizer is selected from sugars and polyalcohols, or derivatives thereof. In particular embodiments, the lyophilizer is selected from monosaccharides, and disaccharides, or mixtures thereof. For example, the lyophilizer may be sucrose, lactulose, trehalose, lactose, glucose, maltose, mannitol, cellobiose, or mixtures thereof.

The compositions disclosed herein can be formulated for administration by, for example, injection. For injection, compositions can be formulated as aqueous solutions, such as in buffers including Hanks' solution, Ringer's solution, or physiological saline, or in culture media, such as Iscove's Modified Dulbecco's Medium (IMDM). The aqueous solutions can include formulatory agents such as suspending, stabilizing, and/or dispersing agents. Alternatively, the compositions can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid compositions such as powders, capsules and tablets, suitable excipients include binders (gum tragacanth, acacia, cornstarch, gelatin), fillers such as sugars, e.g. lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of nanoparticles and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one nanoparticle. Various sustained-release materials have been established and are well known by those of ordinary skill in the art. Sustained-release systems may, depending on their chemical nature, release nanoparticles following administration for a few weeks up to over 100 days. Depot preparations can be administered by injection; parenteral injection; instillation; or implantation into soft tissues, a body cavity, or occasionally into a blood vessel with injection through fine needles.

Depot compositions can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, acetone, dichloromethane, chloroform, ethyl acetate, triacetin, N-methyl pyrrolidone, tetrahydrofuran, phenol, or combinations thereof) can alter nanoparticle size and structure in order to modulate release characteristics. Other useful solvents include water, ethanol, dimethyl sulfoxide (DMSO), N-methyl-2-pyrrolidone (NMP), methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, Pluronics® (e.g., F68/F127), polysorbates (e.g. P80), Span® (Croda Americas, Wilmington, Delaware), poly(vinyl alcohol) (PVA), cholic acid, Brij® (Croda Americas, Wilmington, Delaware), sucrose acetate isobutyrate (SAIB), salts, and buffers.

In particular embodiments, surfactants include a biocompatible substance with amphiphilic molecules which localize to nanoparticle surfaces. In particular embodiments, surfactants includes molecules that promote the adsorption of proteins whose receptors are over-expressed in brain endothelial cells

Excipients that partition into the external phase boundary of nanoparticles such as surfactants including polysorbates, dioctylsulfosuccinates, poloxamers, PVA, can also alter properties including nanoparticle stability and erosion rates, hydration and channel structure, interfacial transport, and kinetics in a favorable manner.

Additional processing of the disclosed sustained release depot compositions can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

Particular embodiments include formulation of nanoparticles within hydrogel compositions. Exemplary hydrogels include collagen hydrogels; type I collagen, fibrin, or a mixture thereof cross-linked, as the cross-linked state of these molecules in vivo; type I collagen hydrogels naturally cross-linked by lysyl oxidase-derived aldimine bonds (Sabeh et al., (2009) J Cell Biol 185:11-19); or other synthetic hydrogels as described in, for example, Rowe & Weiss (2008) Trends Cell Biol 18:560-574; Rowe & Weiss (2009) Annu Rev Cell Dev Biol 25:567-595; Egeblad et al., (2010) Curr Opin Cell Biol 22:697-706; Harunaga & Yamada (2011) Matrix Biol 30:363-368; Willis et al., (2013) J Microsc 251:250-260; and Gill et al. (2012) Cancer Res 72:6013-6023. In particular embodiments, a hydrogel refers to a network of polymer chains that are hydrophilic in which water or an aqueous medium is the dispersion medium. Particular embodiments may utilize a zwitterionic polymer as described in WO2016/040489.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and compositions are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.

In particular embodiments, the compositions include nanoparticles of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

In particular embodiments, nanoparticles within a combination therapy are formulated into separate individual compositions. In particular embodiments, nanoparticle within a combination therapy may be formulated into compositions together. When formulated together, the nanoparticles may be included in the same amounts or in different amounts or ratios. For example, if two types of nanoparticles are provided, these compositions could be included in the following exemplary ratios: 1:1, 2:1, 1:2, 5:1, 1:5, 10:1, 1:10, etc. If three types of nanoparticles are provided, these compositions could be included in the following exemplary ratios: a 1:1:1 ratio, 2:1:1 ratio, 1:2:1 ratio, 1:1:2 ratio, 5:1:1 ratio, 1:5:1 ratio, 1:1:5 ratio, 10:1:1 ratio, 1:10:1 ratio, 1:1:10 ratio, 2:2:1 ratio, 1:2:2 ratio, 2:1:2 ratio, 5:5:1 ratio, 1:5:5 ratio, 5:1:5 ratio, 10:10:1 ratio, 1:10:10 ratio, 10:1:10 ratio, etc. In particular embodiments, different nanoparticles can deliver different enzymes. In particular embodiments, different nanoparticles may have different release profiles. In particular embodiments, different nanoparticles can have different biodistributions. In particular embodiments, a first nanoparticle includes catalase and a second nanoparticle includes an anti-inflammatory enzyme, an excitotoxicity-altering enzyme, or an antioxidative enzyme. In particular embodiments, a first nanoparticle includes lysozyme and a second nanoparticle includes an anti-inflammatory enzyme, an excitotoxicity-altering enzyme, or an antioxidative enzyme. In particular embodiments, a first nanoparticle releases an enzyme at a first time after administration and a second nanoparticle releases an enzyme at a second time after administration. In particular embodiments, a first nanoparticle delivers enzyme to the whole brain and a second nanoparticle delivers enzyme to the brain parenchyma. If one nanoparticle and two external factors are provided, these compositions could be included in the following exemplary ratios: a 1:1:1 ratio, 2:1:1 ratio, 1:2:1 ratio, 1:1:2 ratio, 5:1:1 ratio, 1:5:1 ratio, 1:1:5 ratio, 10:1:1 ratio, 1:10:1 ratio, 1:1:10 ratio, 2:2:1 ratio, 1:2:2 ratio, 2:1:2 ratio, 5:5:1 ratio, 1:5:5 ratio, 5:1:5 ratio, 10:10:1 ratio, 1:10:10 ratio, 10:1:10 ratio, etc.

Compositions disclosed herein can be formulated for administration by, for example, injection, infusion, perfusion, or lavage. The compositions disclosed herein can further be formulated for intraperitoneal, intravenous, intradermal, intraarterial, intranodal, intralymphatic, intralesional, intrathecal, intramuscular, intravesicular, oral, nasal, and/or subcutaneous administration and more particularly by intraperitoneal, intravenous, intradermal, intraarterial, intranodal, intralymphatic, intralesional, intrathecal, intramuscular, intravesicular, and/or subcutaneous injection.

(vii) Methods of Use. Methods disclosed herein include treating subjects (humans, veterinary animals (dogs, cats, reptiles, birds, etc.), livestock (horses, cattle, goats, pigs, chickens, etc.) and research animals (monkeys, rats, mice, fish, etc.)) with compositions disclosed herein. Treating subjects includes delivering therapeutically effective amounts. Therapeutically effective amounts include those that provide effective amounts, prophylactic treatments, and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to the assessment of acute neurological injury progression, neurodegenerative development, systemic oxidative stress burden, lysosomal storage disease progression, metabolic deficiency, or progression of other enzyme-deficient disorders.

A “prophylactic treatment” includes a treatment administered to a subject who does not display signs or symptoms of injury or disease or displays only early signs or symptoms of injury or disease such that treatment is administered for the purpose of diminishing or decreasing the risk of developing injury or disease further. Thus, a prophylactic treatment functions as a preventative treatment against injury or disease or the worsening of injury or disease. In particular embodiments, prophylactic treatments reduce, delay, or prevent the development of injury or disease.

A “therapeutic treatment” includes a treatment administered to a subject who displays symptoms or signs of injury or disease and is administered to the subject for the purpose of diminishing or eliminating those signs or symptoms of injury or disease. The therapeutic treatment can reduce, control, or eliminate the presence or activity of injury or disease and/or reduce control or eliminate side effects of injury or disease.

Function as an effective amount, prophylactic treatment, or therapeutic treatment are not mutually exclusive, and in particular embodiments, administered dosages may accomplish more than one treatment type.

In certain examples, therapeutically effective amounts treat a neurological condition including an acute neurological injury or a chronic neurodegenerative condition. In particular embodiments, acute neurological injury includes encephalopathy, cardiac arrest, and/or traumatic brain injury. In particular embodiments, encephalopathy includes neonatal hypoxic-ischemic encephalopathy. In particular embodiments, a chronic neurodegenerative condition includes multiple sclerosis, amyotrophic lateral sclerosis, Huntington's disease, Alzheimer's disease, and/or Parkinson's disease.

Acute neurological injury such as neonatal hypoxic-ischemic encephalopathy can result in seizures, breathing problems, hypotonia, and organ problems. Prolonged convulsive seizures can be fatal and lead to substantial brain damage. A therapeutic treatment can reduce, control, or eliminate the occurrence of seizures, breathing problems, hypotonia, and organ problems.

Chronic neurodegenerative conditions can result in movement disorders, pain, vision loss, muscle weakness, cognitive disorders, and/or psychiatric symptoms. The therapeutic treatment can reduce, control, or eliminate the presence or activity of the movement disorder, pain, vision loss, muscle weakness, cognitive disorder, and/or psychiatric symptom and/or reduce control or eliminate side effects of the movement disorder, pain, vision loss, muscle weakness, cognitive disorder, and/or psychiatric symptom.

In particular embodiments, methods to determine the efficacy of the treatments using constructs disclosed herein will be measured before treatment, during the first year after treatment, and at other times. In particular embodiments, efficacy of the treatments using constructs disclosed herein will be determined to be effective if the evaluated measurements can be maintained at a normal, non-disorder level, reduced to a non-disorder level, or reduced such that it is still elevated compared to a non-disorder individual, but is still less than the level which would be expected in an individual without treatment.

Therapeutically effective amounts can be assessed using medical and developmental tests. Medical tests can include electroencephalogram (EEG), magnetic resonance imaging (MRI), computed tomography (CT), FDOPA Positron Emission Tomography (PET) putamen-specific radioactivity uptake values, measurement of biomarkers, determination of brain and ventricular volumes, and cerebrospinal fluid (CSF) neurotransmitter metabolite values. Developmental tests include standard evaluations such as the Unified Huntington's Disease Ratings Scale (UHDRS), the Prognostic Index for Huntington's Disease, the Peabody Developmental Motor Scale (PDMS-II), Alberta Infant Motor Scale (AIMS), Bayley Scales of Infant and Toddler Development®-Third Edition (Bayley-III), or the Comprehensive Developmental Inventory for Infants and Toddlers (CDIIT).

In certain examples, therapeutically effective amounts treat a non-neurological condition including an oxidative stress burden, a lysosomal storage disease, or a metabolic deficiency. In particular embodiments, oxidative stress burden is due to diabetes (e.g., type 2 diabetes). In particular embodiments, lysosomal storage diseases include aspartylglucosaminuria, Batten disease, cystinosis, Fabry disease, Gaucher disease, Pompe disease, Sandhoff disease, metachromatic leukodystrophy, mucolipidosis, Niemann-Pick disease, or Schindler disease. In particular embodiments, metabolic deficiencies include Galactosemia, Maple syrup urine disease, phenylketonuria, glycogen storage disease, Friedreich ataxia, Zellweger syndrome, adrenoleukodystrophy, Wilson disease, hemochromatosis, methylmalonic acidemia, propionic acidemia, ornithine transcarbamylase deficiency, or citrullinemia.

The therapeutic treatment can reduce, control, or eliminate the presence or activity of the symptoms of non-neurological conditions. Symptoms of a lysosomal storage disease include seizures, difficulty breathing, joint stiffness, pain, intellectual and physical developmental delay, anemia, and problems with hearing and vision. Symptoms of metabolic deficiencies include high blood pressure, high triglycerides, obesity or failure to gain weight, heart disease, stroke, diabetes, fatigue, fluid buildup, muscle stiffness, lethargy, hypotonia, or vomiting.

Efficacy of treatment can be measured by measuring blood pressure, blood panels, weight measurement, echocardiogram, a nuclear medicine scan, computerized tomography, and magnetic resonance imaging.

Therapeutically effective amounts can be confirmed by observing a reduction in one or more symptoms of injury or disease such as enzyme activity or oxidative stress burden.

For administration, therapeutically effective amounts (also referred to herein as doses) can be initially estimated based on results from in vitro assays and/or animal model studies. Such information can be used to determine useful doses in subjects of interest more accurately. The actual dose amount administered to a particular subject can be determined by a physician, veterinarian, or researcher, taking into account parameters such as physical and physiological factors including target, body weight, stage of injury or disease, the severity of injury or disease, previous or concurrent therapeutic interventions, idiopathy of the subject and route of administration.

Useful enzyme doses can range from 0.1 to 150 mg/kg or from 2.5-5 mg/kg. Particularly useful enzyme can include 0.2 mg/kg, 0.4 mg/kg, 1.8 mg/kg, 2.6 mg/kg, 2.5 mg/kg, 5 mg/kg, 20 mg/kg, and 50 mg/kg. In certain examples, doses do not exceed 150 mg/kg.

Useful polymer doses can be at enzyme to polymer ratios of 1:5 up to 1:20. A polymer dose can range from 0.5 to 500 mg/kg or from 10-250 mg/kg. Tolerated doses will depend on the polymer used. In particular embodiments, useful doses of PLGA is less than 500 mg/kg. In particular embodiments, 80% of the maximum tolerated dose of polystyrene (PS) nanoparticles is 150 mg/kg. In particular embodiments, useful doses of polymer is less than 250 mg/kg.

One of the benefits of the formulation described herein is that it increases the amount of active enzyme that can be delivered for the same amount of polymer. Polymer doses are limited by potential immunogenic responses and the volume of administration available. For example, in neonatal animal models a limited volume can be administered to the neonatal animal. With a fixed volume of solution, increasing active enzyme within the solution changes the viscosity of the solution. Improving the ability to capture more active enzyme in a nanoparticle allows for a decrease in total material dose in vivo.

The pharmaceutical composition may be in unit dosage forms suitable for single administration of precise dosages.

Therapeutically effective amounts can be achieved by administering single or multiple doses during the course of a treatment regimen. Dosing schedules may also be used given the goals of a therapy (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

The compositions described herein can be administered by any method that enables delivery of the nanoparticles to the site of action. Routes of administration include intravenous, intradermal, intraarterial, intraparenteral, intranasal, intralesional, intramuscular, oral, subcutaneous, or infusion. In particular embodiments, administration includes oral routes, intraduodenal routes, and parenteral injection (including intravenous, subcutaneous, intramuscular, intravascular, or infusion).

(viii) Kits. Also provided herein are kits which can provide components for the manufacture of enzyme-loaded nanoparticles disclosed herein and/or their use in research or treatment as described elsewhere herein. The kit may include material(s), which may be desirable from a user standpoint, such as enzymes, polymers, buffer(s), diluent(s), standard(s), and/or other material useful in nanoparticle preparation, administration, or conducting any other step of the methods described herein.

In particular embodiments, a kit includes a polymer, a hydrophobic ion, an enzyme, and solvents. In particular embodiments, a kit includes a PLGA-PEG polymer, dextran sulfate, an enzyme, and solvents.

The kit according to the present disclosure may also include instructions for carrying out the method. Instructions included in the kit of the present disclosure may be affixed to packaging material or may be included as a package insert. While instructions are typically written or printed materials, they can be any medium capable of storing such instructions and communicating them to an end user is contemplated by this disclosure. Such media include electronic storage media (e.g., magnetic discs, tapes, cartridges, chips), optical media (e.g., CD ROM), and the like. As used herein, the term “instructions” can include the address of an internet site which provides instructions.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (ix) Exemplary Embodiments

1. A method of preparing a hydrophobic ion pairing complex for nanoparticle formation, the method including:
   mixing, within a solution, dextran sulfate and an enzyme to form a hydrophobic ion pairing complex within the solution;
   centrifuging the solution to separate the hydrophobic ion pairing complex from the solution; and
   collecting the separated hydrophobic ion pairing complex such that the enzyme retains activity,
   thereby preparing the hydrophobic ion pairing complex for nanoparticle formation.
2. A method of preparing enzyme-loaded nanoparticles, the method including: mixing, within a solution, dextran sulfate and an enzyme to form a hydrophobic ion pairing complex within the solution;
   centrifuging the solution to separate the hydrophobic ion pairing complex from the solution;
   collecting the separated hydrophobic ion pairing complex such that the enzyme retains activity;
   mixing a polymer with the collected hydrophobic ion pairing complex within a solvent to form a mixture;
   adding the mixture to an aqueous solution to form the enzyme-loaded nanoparticles; and separating the formed enzyme-loaded nanoparticles from the aqueous solution, thereby preparing enzyme-loaded nanoparticles.
3. A method of preparing a hydrophobic ion pairing complex, the method including: mixing, within a solution, a hydrophobic ion and a first enzyme to form a hydrophobic ion pairing complex; and
   separating the hydrophobic ion pairing complex from the solution,
   thereby preparing the hydrophobic ion pairing complex,
   wherein the hydrophobic ion includes dextran sulfate, taurocholic acid, sodium dodecyl sulfate, 1-hydroxy-2-naphthoic acid, 2-naphthalene sulfonic acid, alginic acid, arginine-hexadecanoyl ester, arginine-nonyl ester, benethamine, brilliant blue FCF, cetrimonium bromide (CTAB), chitosan, chlorhexidine, cholesteryl hemisuccinate, cholic acid, CM-PEG56, dimyristoyl phosphatidyl glycerol, dioleoyl phosphatidic acid (DOPA), docosahexaenoic acid, docusate sodium, hexadecylphosphate, hyaluronic acid, laurylamine, linoleic acid, losartan, maprotiline, N α-deoxycholyl-L-lysyl-methylester, N,N′-dibenzylethylene diamine (DBDA), N,N-dimethyldodecyl amine (DDA), N,N-dimethylhexyl amine, N,N-dimethyloctadecyl amine, N,N-dipalmitoyl-L-lysine, oleic acid, pamoic acid, sodium acetate, sodium alginate, sodium cholesteryl sulfate, sodium decanesulfonate, sodium decanoate, sodium deoxycholate, sodium docusate, sodium dodecyl benzenesulfonate, sodium laurate, sodium oleate, sodium stearate, sodium stearoyl glutamate, sodium stearyl sulfate, sodium taurodeoxycholate, sodium tetradecyl sulfate, sodium tripolyphosphate, stearylamine, tetrabutylammonium bromide, tetraheptylammonium bromide, tetrahexylammonium bromide, tetraoctylammonium bromide, tetrapentylammonium bromide, triethylamine, or vitamin E succinate;
   and wherein the enzyme retains activity within the hydrophobic ion pairing complex.
4. The method of embodiment 3, wherein the mixing includes vortexing.
5. The method of embodiments 3 or 4, wherein the separating uses centrifugation.
6. The method of any of embodiments 3-5, wherein the first enzyme has a molecular weight between 10 kDa and 300 kDa.
7. The method of any of embodiments 3-6, wherein the first enzyme has a molecular weight of 10 kDa to 50 kDa.
8. The method of any of embodiments 3-7, wherein the first enzyme has a molecular weight of 14 kDa.
9. The method of any of embodiments 3-7, wherein the first enzyme has a molecular weight of 50 kD at 150 kDa.
10. The method of any of embodiments 3-7, wherein the first enzyme has a molecular weight of 150 kDa to 300 kDa.
11. The method of any of embodiments 3-10, wherein the first enzyme has a molecular weight of 240 kDa.
12. The method of any of embodiments 3-11, wherein the first enzyme includes catalase, lysozyme, chymotrypsin, trypsin, serratiopeptidase, bromelain, ficin, papain, serrapeptase, superoxide dismutase (SOD), glutamate cysteine ligase (GCL), or glutathione synthase (GS).
13. The method of any of embodiments 3-12, wherein the first enzyme includes catalase.
14. The method of any of embodiments 3-12, wherein the first enzyme includes lysozyme.
15. The method of any of embodiments 3-12, wherein the first enzyme includes superoxide dismutase (SOD), catalase (CAT), glutathione peroxidase (GPx), aldehyde oxidase, glyoxalase, myeloperoxidase, nitric oxide synthase, and/or sphingomyelin phosphodiesterase.
16. The method of any of embodiments 3-12, wherein the first enzyme includes glucocerebrosidase, sphingomyelin cholinephosphohydrolase, acid sphingomyelinase, acid alpha-glucosidase, aspartylglucosaminidase, alpha-galactosidase A, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, and/or acid lipase.
17. The method of any of embodiments 3-12, wherein the first enzyme includes glucose-6-phosphatase, fructose-1,6-bisphosphatase, tibose-phosphate isomerase, transaldolase, succinate dehydrogenase, glucose-6-phosphate dehydrogenase, fumarase, pyruvate dehydrogenase complex, and/or N-acetylglutamate synthetase.
18. The method of any of embodiments 3-17, wherein the first enzyme retains activity following administration to a subject.
19. The method of any of embodiments 3-18, wherein the first enzyme retains 60%, 65%, 70%, 75%, 80%, 85%, 90%, 95%, or 100% activity compared to a benchmark enzyme activity.
20. The method of embodiment 19, wherein the benchmark enzyme activity is determined by active units per mass according to the enzyme's manufacturer.
21. The method of any of embodiments 3-20, further including mixing a second enzyme within the solution.
22. The method of embodiment 21, wherein the second enzyme is catalase.
23. The method of embodiment 21, wherein the second enzyme is lysozyme.
24. The method of embodiment 21, wherein at least one of the first enzyme or the second enzyme is an anti-inflammatory enzyme, an excitotoxicity-altering enzyme, or an antioxidative enzyme.
25. The method of embodiment 24, wherein the anti-inflammatory enzyme includes chymotrypsin, trypsin, serratiopeptidase, bromelain, ficin, papain, or serrapeptase.
26. The method of embodiment 24, wherein the excitotoxicity-altering enzyme includes glutamate decarboxylase, glutamate dehydrogenase, pyrroline-5-carboxylate synthase (P5CS), serum glutamate oxaloacetate transaminase (AST), or serum glutamate pyruvate transaminase (ALT).
27. The method of embodiment 24, wherein the antioxidative enzyme includes superoxide dismutase (SOD), glutamate cysteine ligase (GCL), or glutathione synthase (GS).
28. The method of any of embodiments 3-27, wherein the solution has a pH ranging from pH 2.0 to 10.5.
29. The method of any of embodiments 3-28, wherein the solution is a buffering agent including phosphate buffer, a citrate buffer, or an acetate buffer.
30. The method of any of embodiments 3-29, wherein the solution is a phosphate buffer.
31. The method of any of embodiments 3-30, wherein the hydrophobic ion is dextran sulfate.
32. The method of any of embodiments 3-31, wherein the method further includes lyophilizing the hydrophobic ion pairing complex.
33. The method of any of embodiments 3-32, wherein the method further includes incorporating the hydrophobic ion pairing complex into a polymeric nanoparticle.
34. The method of any of embodiments 3-33, wherein the hydrophobic ion pairing complex has a molar ratio of at least 0.5 of hydrophobic ion to enzyme.
35. The method of any of embodiments 3-34, wherein the hydrophobic ion pairing complex has a hydrophobic ion to enzyme charge ratio of 1:1.
36. A method of preparing an enzyme-loaded nanoparticle, the method including:
   mixing a polymer with a hydrophobic ion pairing complex within a solvent to form a mixture, wherein the hydrophobic ion pairing complex includes an enzyme and a hydrophobic ion;
   adding the mixture to an aqueous solution to form the enzyme-loaded nanoparticles; and
   separating the formed enzyme-loaded nanoparticles from the aqueous solution,
   thereby preparing enzyme-loaded nanoparticles.
37. The method of embodiment 36, wherein the hydrophobic ion pairing complexes are prepared according to the method of any of embodiments 3-35.
38. The method of embodiments 36 or 37, wherein the polymer includes a hydrophobic polymer.
39. The method of any of embodiments 36-38, wherein the polymer includes a hydrophobic polymer and polyethylene glycol (PEG).
40. The method of embodiment 39, wherein the hydrophobic polymer includes polylactic co-glycolic acid (PLGA), polylactic acid (PLA), or poly-ε-caprolactone (PCL), ethyl cellulose (EC), polybutylcyanoacrylate (PBCA), polypropylene oxide (PPO), or polyhydroxybutyrate (PHB).
41. The method of embodiments 39 or 40, wherein the hydrophobic polymer is polylactic co-glycolic acid (PLGA).
42. The method of any of embodiments 36-41, wherein the hydrophobic ion is dextran sulfate.
43. The method of any of embodiments 36-42, wherein the enzyme is catalase, lysozyme, chymotrypsin, trypsin, serratiopeptidase, bromelain, ficin, papain, serrapeptase, superoxide dismutase (SOD), glutamate cysteine ligase (GCL), or glutathione synthase (GS).
44. The method of any of embodiments 36-43, wherein the enzyme is catalase.
45. The method of any of embodiments 36-43, wherein the enzyme is lysozyme.
46. The method of any of embodiments 36-42, wherein the enzyme includes superoxide dismutase (SOD), catalase (CAT), glutathione peroxidase (GPx), aldehyde oxidase, glyoxalase, myeloperoxidase, nitric oxide synthase, and/or sphingomyelin phosphodiesterase.
47. The method of any of embodiments 36-42, wherein the enzyme includes glucocerebrosidase, sphingomyelin cholinephosphohydrolase, acid sphingomyelinase, acid alpha-glucosidase, aspartylglucosaminidase, alpha-galactosidase A, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, and/or acid lipase.
48. The method of any of embodiments 36-42, wherein the enzyme includes glucose-6-phosphatase, fructose-1,6-bisphosphatase, tibose-phosphate isomerase, transaldolase, succinate dehydrogenase, glucose-6-phosphate dehydrogenase, fumarase, pyruvate dehydrogenase complex, and/or N-acetylglutamate synthetase.
49. The method of any of embodiments 36-48, wherein the enzyme includes a first enzyme and a second enzyme.
50. The method of embodiment 49, wherein the second enzyme is an anti-inflammatory enzyme, an excitotoxicity-altering enzyme, or an antioxidative enzyme.
51. The method of embodiment 50, wherein the anti-inflammatory enzyme includes chymotrypsin, trypsin, serratiopeptidase, bromelain, ficin, papain, or serrapeptase.
52. The method of embodiment 50, wherein the excitotoxicity-altering enzyme includes enzymes that perform glutamate catalysis.
53. The method of embodiment 50, wherein the antioxidative enzyme includes superoxide dismutase (SOD), glutamate cysteine ligase (GCL), or glutathione synthase (GS).
54. The method of any of embodiments 36-53, wherein the solvent is a water-miscible organic solvent.
55. The method of embodiment 54, wherein the water-miscible organic solvent includes acetone, acetonitrile, dimethylsulfoxide (DMSO), methanol, acetaldehyde, acetic acid, 1,2-Butanediol, 1,3-Butanediol, 1,4-Butanediol, 2-Butoxyethanol, butyric acid, dethanolamine, diethylenetriamine, dimethylformamide, dimethoxyethane, 1,4-Dioxane, ethanol, ethylamine, ethylene glycol, formic acid, furfuryl alcohol, glycerol, methyl diethanolamine, methyl isocyanide, N-Methyl-2-pyrrolidone, 1-Propanol, 1,3-Propanediol, 1,5-Pentanediol, 2-Propanol, propanoic acid, propylene glycol, pyridine, tetrahydrofuran, or triethylene glycol.
56. The method of embodiment 54, wherein the water-miscible organic solvent is acetone.
57. The method of any of embodiments 36-56, wherein the mixing includes vortexing or stirring.
58. The method of any of embodiments 36-57, wherein the adding the mixture to the aqueous solution includes adding the mixture dropwise into the aqueous solution and stirring.
59. The method of any of embodiments 36-58, wherein the aqueous solution includes a surfactant in solution.
60. The method of embodiment 59, wherein the surfactant includes a polysorbate, a poloxamer, a nonionic surfactant, or an anionic surfactant.
61. The method of embodiment 60, wherein the polysorbate is polysorbate 80.
62. The method of embodiment 60, wherein the poloxamer is poloxamer 188 (F68) or poloxamer 407 (F127).
63. The method of embodiment 60, wherein the nonionic surfactant is polyvinyl alcohol (PVA).
64. The method of embodiment 60, wherein the anionic surfactant is cholic acid.
65. The method of any of embodiments 36-64, wherein the hydrophobic ion pairing complex is a solid and/or is dissolved in a water miscible organic solvent.
66. The method of embodiment 65, wherein the water-miscible organic solvent includes acetone, acetonitrile, dimethylsulfoxide (DMSO), methanol, acetaldehyde, acetic acid, 1,2-Butanediol, 1,3-Butanediol, 1,4-Butanediol, 2-Butoxyethanol, butyric acid, dethanolamine, diethylenetriamine, dimethylformamide, dimethoxyethane, 1,4-Dioxane, ethanol, ethylamine, ethylene glycol, formic acid, furfuryl alcohol, glycerol, methyl diethanolamine, methyl isocyanide, N-Methyl-2-pyrrolidone, 1-Propanol, 1,3-Propanediol, 1,5-Pentanediol, 2-Propanol, propanoic acid, propylene glycol, pyridine, tetrahydrofuran, or triethylene glycol.
67. The method of any of embodiments 36-66, wherein the solvent is a water-immiscible organic solvent.
68. The method of embodiment 67, wherein the water-immiscible organic solvent includes dichloromethane, chloroform, or acyl acetate.
69. The method of embodiments 67 or 68, wherein the water-immiscible organic solvent includes dichloromethane.
70. The method of any of embodiments 36-69, wherein the mixing includes homogenization or sonication.
71. The method of any of embodiments 36-70, wherein the adding the mixture to the aqueous solution includes pouring the mixture into the aqueous solution and stirring.
72. The method of any of embodiments 36-71, wherein the aqueous solution includes a surfactant in solution.
73. The method of embodiment 72, wherein the surfactant includes a polysorbate, a poloxamer, a nonionic surfactant, or an anionic surfactant.
74. The method of embodiment 73, wherein the polysorbate is polysorbate 80.
75. The method of embodiment 73, wherein the poloxamer is poloxamer 188 (F68) or poloxamer 407 (F127).
76. The method of embodiment 73, wherein the nonionic surfactant is polyvinyl alcohol (PVA).
77. The method of embodiment 73, wherein the anionic surfactant is cholic acid.
78. The method of any of embodiments 36-77, wherein the separating of the nanoparticles is by centrifuging.
79. The method of any of embodiments 36-78, wherein the separating of the nanoparticles is by washing and ultracentrifugation with a buffer.
80. The method of embodiment 78, wherein the buffer is phosphate buffer.
81. The method of any of embodiments 36-80, further including resuspending the separated nanoparticles in deionized water.
82. A nanoparticle including (i) a polymer and (ii) a hydrophobic ion pairing complex including a hydrophobic ion and an enzyme.
83. The nanoparticle of embodiment 82, wherein the polymer includes a hydrophobic polymer.
84. The nanoparticle of embodiments 82 or 83, wherein the polymer includes the hydrophobic polymer and a polyethylene glycol (PEG).
85. The nanoparticle of embodiments 83 or 84, wherein the hydrophobic polymer includes polylactic co-glycolic acid (PLGA), polylactic acid (PLA), polyglycolic acid (PGA), poly-ε-caprolactone (PCL), ethyl cellulose (EC), polybutylcyanoacrylate (PBCA), polypropylene oxide (PPO), or polyhydroxybutyrate (PHB).
86. The nanoparticle of any of embodiments 83-85, wherein the hydrophobic polymer is polylactic co-glycolic acid (PLGA).
87. The nanoparticle of any of embodiments 82-86, wherein the polymer includes PLGA and PEG, the hydrophobic ion includes dextran sulfate, and the enzyme includes catalase.
88. The nanoparticle of any of embodiments 82-86, wherein the polymer includes PLGA and PEG, the hydrophobic ion includes dextran sulfate, and the enzyme includes lysozyme.
89. The nanoparticle of any of embodiments 82-87, wherein the enzyme has a molecular weight ranging between 10 kDa and 300 kDa.
90. The nanoparticle of any of embodiments 82-89, wherein the enzyme has a molecular weight of 10 kDa to 50 kDa.
91. The nanoparticle of any of embodiments 82-90, wherein the enzyme has a molecular weight of 14 kDa.
92. The nanoparticle of any of embodiments 82-89, wherein the enzyme has a molecular weight of 50 kDa to 150 kDa.
93. The nanoparticle of any of embodiments 82-89 wherein the enzyme has a molecular weight of 150 kDa to 300 kDa.
94. The nanoparticle of embodiment 93, wherein the enzyme has a molecular weight of 240 kDa.
95. The nanoparticle of any of embodiments 82-94, wherein the enzyme is positively charged at a pH when the hydrophobic ion is negatively charged.
96. The nanoparticle of embodiment 95, wherein the pH is 2.0.
97. The nanoparticle of embodiment 95, wherein the pH is 10 to 10.5.
98. The nanoparticle of any of embodiments 82-97, wherein the enzyme retains activity following administration to a subject.
99. The nanoparticle of any of embodiments 82-98, wherein the enzyme retains 60%, 65%, 70%, 75%, 80%, 85%, 90%, 95%, or 100% activity compared to a benchmark enzyme activity.
100. The nanoparticle of embodiment 99, wherein the benchmark enzyme activity is determined by active units per mass according to the enzyme's manufacturer.
101. The nanoparticle of any of embodiments 82-100, wherein the enzyme is catalase, lysozyme, chymotrypsin, trypsin, serratiopeptidase, bromelain, ficin, papain, serrapeptase, superoxide dismutase (SOD), glutamate cysteine ligase (GCL), or glutathione synthase (GS).
102. The nanoparticle of any of embodiments 82-101, wherein the enzyme is catalase.
103. The nanoparticle of any of embodiments 82-101, wherein the enzyme is lysozyme.
104. The nanoparticle of any of embodiments 82-100, wherein the enzyme includes superoxide dismutase (SOD), catalase (CAT), glutathione peroxidase (GPx), aldehyde oxidase, glyoxalase, myeloperoxidase, nitric oxide synthase, and/or sphingomyelin phosphodiesterase.
105. The nanoparticle of any of embodiments 82-100, wherein the enzyme includes glucocerebrosidase, sphingomyelin cholinephosphohydrolase, acid sphingomyelinase, acid alpha-glucosidase, aspartylglucosaminidase, alpha-galactosidase A, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, and/or acid lipase.
106. The nanoparticle of any of embodiments 82-100, wherein the enzyme includes glucose-6-phosphatase, fructose-1,6-bisphosphatase, tibose-phosphate isomerase, transaldolase, succinate dehydrogenase, glucose-6-phosphate dehydrogenase, fumarase, pyruvate dehydrogenase complex, and/or N-acetylglutamate synthetase.
107. The nanoparticle of any of embodiments 82-100, wherein the enzyme includes at least two enzymes.
108. The nanoparticle of embodiment 107, wherein at least one of the at least two enzymes is catalase.
109. The nanoparticle of embodiments 107 or 108, wherein at least one of the at least two enzymes is lysozyme.
110. The nanoparticle of any of embodiments 107-109, wherein at least one of the at least two enzymes is an anti-inflammatory enzyme, an excitotoxicity-altering enzyme, or an antioxidative enzyme.
111. The nanoparticle of embodiment 110, wherein the anti-inflammatory enzyme includes chymotrypsin, trypsin, serratiopeptidase, bromelain, ficin, papain, or serrapeptase.
112. The nanoparticle of embodiment 110, wherein the excitotoxicity-altering enzyme includes glutamate decarboxylase, glutamate dehydrogenase, pyrroline-5-carboxylate synthase (P5CS), serum glutamate oxaloacetate transaminase (AST), or serum glutamate pyruvate transaminase (ALT).
113. The nanoparticle of embodiment 110, wherein the antioxidative enzyme includes superoxide dismutase (SOD), glutamate cysteine ligase (GCL), or glutathione synthase (GS).
114. The nanoparticle of any of embodiments 82-113, wherein the hydrophobic ion includes dextran sulfate, taurocholic acid, sodium dodecyl sulfate, 1-hydroxy-2-naphthoic acid, 2-naphthalene sulfonic acid, alginic acid, arginine-hexadecanoyl ester, arginine-nonyl ester, benethamine, brilliant blue FCF, cetrimonium bromide (CTAB), chitosan, chlorhexidine, cholesteryl hemisuccinate, cholic acid, CM-PEG56, dimyristoyl phosphatidyl glycerol, dioleoyl phosphatidic acid (DOPA), docosahexaenoic acid, docusate sodium, hexadecylphosphate, hyaluronic acid, laurylamine, linoleic acid, losartan, maprotiline, N α-deoxycholyl-L-lysyl-methylester, N,N′-dibenzylethylene diamine (DBDA), N,N-dimethyldodecyl amine (DDA), N,N-dimethylhexyl amine, N,N-dimethyloctadecyl amine, N,N-dipalmitoyl-L-lysine, oleic acid, pamoic acid, sodium acetate, sodium alginate, sodium cholesteryl sulfate, sodium decanesulfonate, sodium decanoate, sodium deoxycholate, sodium docusate, sodium dodecyl benzenesulfonate, sodium laurate, sodium oleate, sodium stearate, sodium stearoyl glutamate, sodium stearyl sulfate, sodium taurodeoxycholate, sodium tetradecyl sulfate, sodium tripolyphosphate, stearylamine, tetrabutylammonium bromide, tetraheptylammonium bromide, tetrahexylammonium bromide, tetraoctylammonium bromide, tetrapentylammonium bromide, triethylamine, or vitamin E succinate.
115. The nanoparticle of any of embodiments 82-114, wherein the hydrophobic ion is dextran sulfate.
116. The nanoparticle of any of embodiments 82-115, further including a surfactant.
117. The nanoparticle of embodiment 116, wherein the surfactant includes a polysorbate, a poloxamer, a nonionic surfactant, or an anionic surfactant.
118. The nanoparticle of embodiment 117, wherein the polysorbate is polysorbate 80 (P80).
119. The nanoparticle of embodiment 117, wherein the poloxamer is poloxamer 188 (F68) or poloxamer 407 (F127).
120. The nanoparticle of embodiment 117, wherein the nonionic surfactant is polyvinyl alcohol (PVA).
121. The nanoparticle of embodiment 117, wherein the anionic surfactant is cholic acid.
122. The nanoparticle of any of embodiments 82-121, wherein the nanoparticle does not require cell targeting ligands.
123. The nanoparticle of any of embodiments 82-122, wherein the nanoparticle has a hydrodynamic diameter ranging from 40 nm to 140 nm.
124. The nanoparticle of any of embodiments 82-123, wherein the nanoparticle has a hydrodynamic diameter ranging from 100 nm to 130.6 nm.
125. The nanoparticle of any of embodiments 82-123, wherein the nanoparticle has a hydrodynamic diameter ranging from 50 nm to 70 nm.
126. The nanoparticle of any of embodiments 82-125, wherein the nanoparticle has a mean surface charge (ζ-potential) ranging from −10 mV to −0.5 mV.
127. The nanoparticle of any of embodiments 82-126, wherein the nanoparticle has a mean surface charge (ζ-potential) ranging from −7 mV to −2.1 mV.
128. The nanoparticle of any of embodiments 82-127, wherein the nanoparticle has a polydispersity index ranging from 0.05 to 0.3.
129. The nanoparticle of any of embodiments 82-128, wherein the nanoparticle has a polydispersity index ranging from 0.08 to 0.25.
130. A method of treating a condition including administering a therapeutically effective amount of the nanoparticle of any of embodiments 82-129 to a subject in need thereof, wherein the nanoparticle includes an enzyme.
131. The method of embodiment 130, wherein the condition is a neurological condition.
132. The method of embodiment 131, wherein the neurological condition is an acute neurological injury or a chronic neurodegenerative condition.
133. The method of embodiment 132, wherein the acute neurological injury is an encephalopathy.
134. The method of embodiment 133, wherein the encephalopathy is neonatal hypoxic-ischemic encephalopathy.
135. The method of embodiment 132, wherein the chronic neurodegenerative condition includes multiple sclerosis, amyotrophic lateral sclerosis, Huntington's disease, Alzheimer's disease, or Parkinson's disease.
136. The method of embodiment 130, wherein the condition is a non-neurological condition.
137. The method of embodiment 136, wherein the non-neurological condition includes oxidative stress burden, lysosomal storage disease, or a metabolic deficiency.
138. The method of embodiment 137, wherein the oxidative stress burden is due to type 2 diabetes.
139. The method of any of embodiments 130-138, wherein the subject is in need thereof following cardiac arrest.
140. The method of any of embodiments 130-139, wherein the administering is intravenous, intraperitoneal, intramuscular, oral, or nasal administration.
141. The method of any of embodiments 130-140, wherein the administering is intravenous.
142. The method of any of embodiments 130-141, wherein the subject is a mammal.
143. The method of embodiment 142, wherein the mammal is a human.
144. The method of embodiment 143, wherein the human is a neonate.
145. The method of embodiment 143, wherein the human is an infant or a child.
146. The method of embodiment 143, wherein the human is an adult.
147. The method of any of embodiments 130-146, wherein the enzyme retains activity following administration to a subject at least until a therapeutic objective is achieved.
148. The method of any of embodiments 130-147, wherein the enzyme retains 60%, 65%, 70%, 75%, 80%, 85%, 90%, 95%, or 100% activity compared to a benchmark enzyme activity.
149. The method of embodiment 148, wherein the benchmark enzyme activity is determined by active units per mass according to the enzyme's manufacturer.
150. A method of delivering an enzyme to a central nervous system of a subject including administering the nanoparticle of any of embodiments 82-129 to the subject, wherein the nanoparticle includes an enzyme.
151. The method of embodiment 150, wherein the administering includes intravenous, intraperitoneal, intramuscular, oral, or nasal administration.
152. The method of embodiments 150 or 151, wherein the administering is intravenous.
153. The method of any of embodiments 150-152, wherein the enzyme retains activity following administration to the subject at least until a physiological effect is achieved.
154. The method of embodiment 153, wherein the physiological effect is reduced oxidative burden.
155. The method of embodiment 153, wherein the physiological effect is neuroprotection.
156. The method of any of embodiments 150-155, wherein the enzyme retains 60%, 65%, 70%, 75%, 80%, 85%, 90%, 95%, or 100% activity compared to a benchmark enzyme activity.
157. The method of embodiment 156, wherein the benchmark enzyme activity is determined by active units per mass according to the enzyme's manufacturer.
158. The method of any of embodiments 150-157, wherein the subject is a mammal.
159. The method of embodiment 158, wherein the mammal is a human.
160. The method of embodiment 159, wherein the human is a neonate.
161. The method of embodiment 159, wherein the human is an infant or a child.
162. The method of embodiment 159, wherein the human is an adult.

### (x) Experimental Examples. Experimental Example 1

Abstract. Neonatal hypoxic-ischemic encephalopathy is the leading cause of permanent brain injury in term newborns and currently has no cure. Catalase, an antioxidant enzyme, is a promising therapeutic due to its ability to scavenge toxic reactive oxygen species and improve tissue oxygen status. However, upon in vivo administration, catalase is subject to a short half-life, rapid proteolytic degradation, immunogenicity, and an inability to penetrate the brain. Polymeric nanoparticles can improve pharmacokinetic properties of therapeutic cargo, although encapsulation of large proteins has been challenging. In this example, hydrophobic ion pairing is used as a technique for increasing the hydrophobicity of catalase and driving its subsequent loading into a poly(lactic-co-glycolic acid)-poly(ethylene glycol) (PLGA-PEG) nanoparticle was investigated. Improved formation of catalase-hydrophobic ion complexes with dextran sulfate (DS) compared to sodium dodecyl sulfate (SDS) or taurocholic acid (TA) were found. Molecular dynamics simulations in a model system demonstrated retention of native protein structure after complexation with DS, but not SDS or TA. Using DS-catalase complexes, catalase-loaded PLGA-PEG nanoparticles were developed and their efficacy in the Vannucci model of unilateral hypoxic-ischemic brain injury in postnatal day 10 rats was evaluated. Catalase-loaded nanoparticles retained enzymatic activity for at least 24 h in serum-like conditions, distributed through injured brain tissue, and delivered a significant neuroprotective effect compared to saline and blank nanoparticle controls. These results encourage further investigation of catalase and PLGA-PEG nanoparticle-mediated drug delivery for the treatment of neonatal brain injury.

Introduction. Neonatal hypoxic-ischemic encephalopathy (HIE) is a devastating neurological condition that affects 1.3-4.7 in 1000 live births in the United States (Kurinczuk et al., Early Hum Dev 2010, 86, 329-338; and Wu, et al., Pediatrics 2004, 114, 1584-1590). The current standard-of-care, therapeutic hypothermia (TH), is only clinically implemented in cases of moderate or severe HIE and still leaves more than 30% of infants dead or with severe disability (Shankaran et al., JAMA 2017, 318, 57-67). One strategy to improve neonatal outcomes is to use a pharmaceutical agent to complement the neuroprotective mechanisms of TH. For example, erythropoietin (Epo) has neuroprotective anti-inflammatory and anti-oxidant properties and has been successfully translated from the Vannucci model of HIE in rats to non-human primates and is now in clinical trials (McPherson et al., Neonatology 2007, 91, 36-43; and Traudt et al., Dev Neurosci 2013, 35, 491-503). Another promising therapeutic may be catalase, a large (240 kDa) enzyme that converts the reactive oxygen species (ROS) hydrogen peroxide to water and molecular oxygen. By improving oxygen status, removing ROS, and subsequently suppressing inflammation, catalase has demonstrated efficacy against a range of pathologies including solid tumors (Cheng et al., Acta Biomater 2020, 112, 234-249; and Chen et al., J Control Release 2017, 263, 79-89), inflammatory bowel disease (Zhang et al., Biomaterials 2016, 105, 206-221), and vascular dysfunction (Muro et al., Am J Physiol Cell Physiol 2003, 285, C1339-1347). For neurological applications, catalase can attenuate neuroinflammatory and apoptotic pathways in vitro (Armogida et al., Int J Immunopathol Pharmacol 2011, 24, 735-747; and Wang et al., J Neurochem 2003, 87, 1527-1534), but its application in vivo has been limited due to its short half-life, proteolytic degradation, immunogenicity, and inability to penetrate the brain (Singhal et al., Cell Death Dis 2013, 4, e903; and Haney et al., Nanomedicine (Lond) 2011, 6, 1215-1230).

Nanoparticles serve as vehicles that can improve drug biodistribution and bioavailability. Drug-loaded biodegradable nanoparticles composed of poly(lactic-co-glycolic acid)-poly(ethylene glycol) (PLGA-PEG) have been shown to improve drug solubility, stability, circulation time, release kinetics, and transport to and within the brain parenchyma (Patel et al., Adv Drug Deliv Rev 2012, 64, 701-705; and Joseph et al., Nano Research 2018, 11, 5670-5688). Enzymes can particularly benefit from nanoparticle encapsulation as the polymer matrix provides protection from immune clearance and systemic degradation (Yu et al., J Control Release 2016, 240, 24-37). However, the hydrophilic nature of enzymes limits their encapsulation into the hydrophobic core of PLGA-PEG nanoparticles. Hydrophobic ion pairing (HIP) is a recently-developed technique that increases the lipophilicity of peptides and proteins (Quintanar-Guerrero et al., Pharm Res 1997, 14, 119-127; and Meyer et al., Pharm Res 1998, 15, 188-193). In this technique, complexes are formed by electrostatic interactions between ionizable groups on the protein and an ion-pairing agent. Complexes are reversible and can dissociate in ionic solutions, but they are also lipophilic due to hydrophobic groups on the ion-pairing agent (Gaudana et al., J Drug Deliv 2011, 2011, 458128). HIP has previously been used for PLGA nanoparticle encapsulation of small peptides, antibodies, and proteins as large as bovine serum albumin (BSA, 60 kDa) (Gaudana et al., J Drug Deliv 2011, 2011, 458128; Patel et al., J Microencapsul 2014, 31, 542-550; Song et al., J Control Release 2016, 229, 106-119; and Yang et al., Drug Dev Ind Pharm 2009, 35, 959-968). Complexation of large enzymes such as catalase, and subsequent nanoparticle formulation and evaluation, has not yet been reported.

In this example, the effects of ion-pairing agent, molar ratio, pH, and buffer ion on catalase HIP complexation efficiency were determined. Molecular dynamics (MD) simulations were used to probe the effect of each ion-pairing agent on the protein structure and bring insight into the HIP complexation process. MD simulations can investigate the molecular-scale interactions between enzymes and polymers for drug delivery applications (Samanta and Roccatano, J Phys Chem B 2013, 117, 3250-3257; and Ahmad et al., J R Soc Interface 2010, 7 Suppl 4, S423-433). Using an optimized catalase complex, a PLGA-PEG nanoparticle formulation was developed which provides high catalase activity and protection in degradative conditions. Finally, the efficacy of the catalase-loaded PLGA-PEG nanoparticles were assessed in the Vannucci model of HIE in neonatal rats.

Materials and Methods. Preparation of hydrophobic ion pairing (HIP) complexes. Stocks of each ion-pairing agent were made in deionized (DI) water: 20 mM dextran sulfate (DS, Mᵣ 5000, Millipore Sigma), 250 mM sodium dodecyl sulfate (SDS, Millipore Sigma), and 500 mM taurocholic acid (TA, Millipore Sigma). Phosphate buffer (50 mM) was adjusted to pH 4.7 with 0.1 N HCl and then used to dissolve catalase at a 10 mg/mL concentration. Based on molar ratio, an appropriate volume of ion pairing (IP) agent (less than 25 μL) was slowly added to the catalase solution, spontaneously forming HIP complexes. The solution was vigorously vortexed for 1 min followed by centrifugation at 12,000 rpm for 15 min at 4° C. Uncomplexed catalase in the supernatant was measured by BCA assay. Pelleted HIP complexes were lyophilized into powder and stored at 4° C.

The above procedure was modified appropriately for individual experiments: The catalase solution pH was adjusted to 4.2, 5.2, and 7.0 for the pH variation study. Citrate buffer (10 mM) was used instead of phosphate buffer to determine the effect of buffer ion species. For bovine serum albumin (BSA) complexes, BSA (ThermoFisher) was dissolved in citrate buffer at pH 3.7 prior to the addition of IP agent.

Characterization of catalase binding efficiency and mass by BCA assay. Binding efficiency was measured indirectly by measuring protein concentration in the initial solution and supernatant using the Pierce BCA Protein Assay Kit (ThermoFisher). Following the manufacturer's instructions, 25 μL sample was added to 96-well plate in triplicate on ice. After addition of 200 μL BCA assay working reagent (50:1 reagent A:B), the plate was placed on a shaker plate at 37° C. for 30 min. After 30 min, the plate was placed on ice, and absorbance was measured at 562 nm on a SpectraMax M5 UV-Vis Spectrophotometer (Molecular Devices). Percentage binding efficiency was calculated according to the following equation:

$$\\ Binding efficiency = \frac{( initial - supernatant )}{initial} \times 100 \%$$

For the quantification of catalase mass in nanoparticles, 100 μL of catalase-loaded nanoparticles was combined with 50 μL of 1 M sodium hydroxide (ThermoFisher). The solution was vortexed for 2 s, spun down on a minicentrifuge, and then incubated at 37° C. for 30 min for base-catalyzed hydrolysis of the polylactic-co-glycolic acid (PLGA) polymer to release all loaded catalase. 50 μL phosphate buffered saline (PBS) was then added to neutralize the solution, which was then measured according to the BCA assay kit.

Catalase activity assay. Catalase (catalase from bovine liver, Sigma) was used as a model enzyme due to its facile enzymatic activity measurement using a catalase spectrophotometric assay adapted from Beers and Sizer (Beers, R F and I W Sizer, J Biol Chem 1952, 195, 133-140). A pH 7.0 solution of 0.036% w/w H₂O₂(Sigma) was prepared in 50 mM phosphate buffer with a 240-nm absorbance (A₂₄₀) between 0.48 and 0.52. In an optically clear quartz cuvette (Hellma Analytics), 100 μL of catalase sample was added to 2.9 mL of H₂O₂ solution, mixed via pipetting, and A₂₄₀ was measured at 2s intervals for 3 min using a kinetic spectrometric reading on a SpectraMax M5 UV-Vis Spectrophotometer (Molecular Devices). The active units per mL of catalase solution (freely dissolved or encapsulated in nanoparticles) were calculated using the following equation:

$$\\ Active units / mL = \frac{( 3.45 ) ( dilution factor )}{( time ) \times 0.1}$$

In the equation, 3.45 represents the decomposition of 3.45 μmol H₂O₂ during A₂₄₀ decrease from 0.45 to 0.4 and 0.1 is the mL volume of sample added. This assay measures catalase activity even when the enzyme is encapsulated, due to the ability of H₂O₂ to diffuse throughout the polymer matrix (Dziubla, et al., J Control Release 2005, 102, 427-439). Stock catalase had 2000-5000 AU/mg catalase.

Atomistic BSA/Ion Pairing (IP) Agent MD Simulations. GROMACS 2020.5 (Lindahl et al., GROMACS 2020.5 Source code. Zenodo 2021, doi:10.5281/zenodo.3562495) was used to simulate all BSA/IP agent systems at 298.15 K, 1 bar and in a water/ion medium. BSA structure was taken from the RCSB protein database (PDB code 4F5S). Using an online MD preparation platform, PlayMolecule (Martinez-Rosell, et al., J Chem Inf Model 2017, 57, 1511-1516), BSA structure at pH 3.7 was extracted, where the predicted net charge was +78. AMBER99SB*-ILDNP forcefield (Aliev, et al., Proteins 2014, 82, 195-215) was used for BSA partial charge and topological parameters. GLYCAM-06j-1 forcefield (Kirschner, et al., J Comput Chem 2008, 29, 622-655) was used for DS topological parameters and the general amber forcefield (GAFF) (Wang, et al., J Mol Graph Model 2006, 25, 247-260 and Wang, et al., J Comput Chem 2004, 25, 1157-1174) was used for SDS and TA topological parameters. An α-1,6 linked dodecamer was simulated for the BSA/DS simulations, due to its experimental molecular weight and high percentage of α-1,6 linkages (Yu, et al., Journal of Molecular Structure 2018, 1156, 320-329). Partial atomic charges for each IP agent were ascertained through the residual electrostatic potential fitting method (Bayly, et al., The Journal of Physical Chemistry 1993, 97, 10269-10280), using the Hartee-Fock level with the 6-31G* basis set in Gaussian 09 (Frisch et al., Gaussian 09, Revision E.01. Gaussian, Inc., Wallingford CT, 2009). A three-point (TIP3P) (Mark and Nilsson, The Journal of Physical Chemistry A 2001, 105, 9954-9960) explicit solvent model is used for water, while temperature control was achieved using the modified Berendsen Thermostat (Bussi, et al., The Journal of Chemical Physics 2007, 126, 014101); pressure control was achieved using the Parrinello-Rahman Barostat (Parrinello and Rahman, Journal of Applied Physics 1981, 52, 7182-7190). In order to maximize computational efficiency, the Hydrogen Mass Repartitioning method (Hopkins et al., J Chem Theory Comput 2015, 11, 1864-1874) was used to allow for a 4-femtosecond timestep. Calculation of backbone RMSD was performed using the built-in GROMACS function gmx rms. All production runs were 200 nanoseconds. All systems were packed within a cubic box with a side length of 15 nm using PACKMOL (Martinez, et al., J Comput Chem 2009, 30, 2157-2164) and were charge neutralized with either potassium or chloride ions.

Fraction of surface residues with >95% occupancy. Percent occupancy was calculated by counting the number of frames an IP agent was within 4 angstroms of a BSA amino acid surface amino acid (AA) divided by the total number of frames within the entire trajectory.

$$\\ \% Occupancy = \frac{\begin{matrix}No . of frames that an IP molecule \\ was within 4 Å of a protein residue\end{matrix}}{Total no . of frames in the whole trajectory} \times 100$$

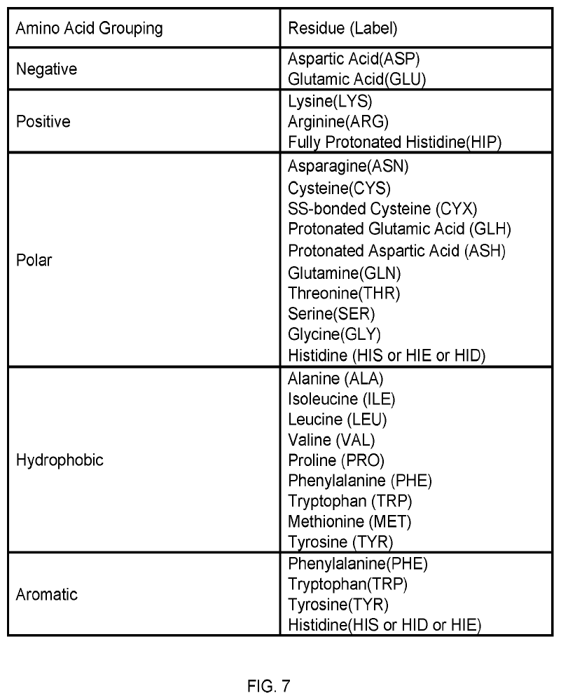

Once occupancy was extracted, residues with >95% occupancy were sub-selected from the total list of residues with a nonzero value of occupancy and were grouped into 5 categories: negative, positive, polar, hydrophobic and aromatic (FIG. 7). The number of residues in each grouping was then normalized by the total number of residues with >95% occupancy to allow for comparison.

$$\\ Fraction of AA with > 95 \% occupancy = \frac{No . of residues in an AA grouping with > 95 \% occupancy}{Total no . of residues with > 95 \% occupancy}$$

Nanoparticle formulation. To formulate nanoparticles by S/O/W (solid in oil in water) emulsion, 1 mL of 25 mg/mL PLGA45k-PEG5k (LA:GA 50:50, Akina) dissolved in dichloromethane (DCM, Fisher Scientific) was added to 1 mg lyophilized catalase HIP complexes. The mixture was emulsified with a Sonic Dismembrator Ultrasonic Processor (Fisher Scientific) using 20 kHz probe sonication at 30% amplitude with 1s on:1s off pulses for 30s on. After adding 4 mL 3% cholic acid (Millipore Sigma) in DI water, the second sonication was performed at 20% amplitude with 1s on:1s off pulses for 30s on. This emulsion was then poured into 25 mL beaker of 1% polysorbate 80 (P80, Millipore Sigma) and stirred for 3 h at 500 rpm to remove the organic solvent. Nanoparticles were collected and washed twice by ultracentrifugation with phosphate buffer at 100,000×g for 25 min. Finally, the nanoparticles were resuspended in 1 mL DI water. Nanoparticles were used immediately or stored at 4° C. for a short time.

To formulate nanoparticles by nanoprecipitation, 1 mg lyophilized catalase or BSA complexes were dissolved in 0.3 mL dimethyl sulfoxide (Millipore Sigma). 25 mg PLGA-PEG was dissolved in 0.7 mL acetone (Fisher Scientific). The two solutions were quickly vortexed together before being added dropwise into 25 mL 1% P80. The remainder of the procedure was as described above. For animal experiments, catalase and BSA nanoparticles were resuspended in sterile PBS.

Nanoparticle characterization. Nanoparticle size and PDI were measured by dynamic light scattering. The ζ-potential was determined using a zeta potential analyzer (NanoSizer Zeta Series, Malvern Instruments, Malvern, UK). Samples were diluted to appropriate concentrations to obtain accurate measurements in 10 mM NaCl at room temperature, pH 7.4, as described previously (Xu, et al., J Control Release 2013, 170, 279-286).

Encapsulated catalase mass was measured by BCA assay kit and catalase activity was measured by activity assay, as described above. To determine catalase protection, nanoparticles were incubated in PBS with 0.2 wt % pronase (pronase from Streptomyces griseus, Sigma). Aliquots were collected at 0 h, 1 h, 2 h, 4 h, and 24 h, and immediately tested for catalase activity. Enzyme activities were calculated as the sample activity at a given timepoint divided by the initial sample activity at 0 h.

Animal experiments and ethics statement. This study was performed in strict accordance with the recommendations in the Guide for the Care and Use of Laboratory Animals of the National Institutes of Health. All of the animals were handled according to approved Institutional Animal Care and Use Committee (IACUC) protocols (#4484-01) associated with Animal Welfare Assurance (#A3464-01) on file with the NIH Office of Laboratory Animal Welfare (OLAW), registered with the United States Department of Agriculture (USDA, certificate #91-R-0001), and accredited by AAALAC International. Every effort was made to minimize suffering. Sprague-Dawley female dams with sex-balanced litters (virus antibody-free CD® (SD) IGS, Charles River Laboratories, Raleigh, NC, USA) were purchased and arrived when pups were postnatal day 5 (P5). The day of birth was defined P0. Before and after the experiment, each dam and her pups were housed under standard conditions with an automatic 12 h light/dark cycle, a temperature range of 20-26° C., and access to standard chow and autoclaved tap water ad libitum. The pups were checked for health daily.

Vannucci model of unilateral hypoxic ischemia (HI) injury in neonatal rats and drug administration. On P10, pups were separated from their dams, weighed and sexed, and randomized to experimental groups. Anesthesia with isoflurane (3-5%) was given in 100% 02 via a nose cone, under a dissecting microscope. The left carotid artery was identified and ligated. Pups were maintained in a temperature-controlled water bath before and after undergoing unilateral ligation of the left carotid artery. After all the animals recovered from anesthesia, they returned to the dams for a minimum of 30 min before placement in a hypoxic chamber in a temperature-controlled water bath. Once rectal temperature in a sentinel animal was stable at 36° C. for 5 min, the chamber was sealed and 8% O₂ (92% N₂) administered at a rate of 2.5 L/min. Once the oxygen concentration within the chamber reached 8%, hypoxia was maintained for 2 hours or until 10% mortality was reached. The end of hypoxia marked the end of the insult (i.e., 0 h timepoint).

At 30 min, 24 h, and 48 h after injury, treatments were administered intraperitoneally. A total of 58 pups (30 males, 28 females) were randomized into 3 separate treatment groups: saline (12 male, 11 female), blank nanoparticles (9 male, 7 female), and catalase nanoparticles (9 male, 10 female). Catalase nanoparticles were dosed at 3300 AU/kg. An equivalent polymer and protein mass was delivered with the blank formulation. Dosage and timing were based on previous investigation of the therapeutic window for pharmacological agents in the Vannucci model (Joseph, et al., Nano Res 2018, 11, 5670-5688; and Kellert, et al., Pediatr Res 2007, 61, 451-455).

Gross injury scoring and area loss. 72 h after injury, animals received an overdose of pentobarbital before transcardiac perfusion with 0.9% saline. Immediately following brain extraction, a photo of each whole brain was taken for gross injury scoring. The brain was then sliced into 3 mm thick slices at the level of the hippocampus and thalamus. Slices were incubated in prewarmed 2,3,5-triphenyltetrazolium chloride (TTC, Fisher Scientific) for 10 min at 37° C. The slices were then fixed in 10% neutral buffered formalin for 24 h before being imaged for area loss measurement.

Both gross injury and area loss scoring was conducted by two independent individuals who were blinded to group allocation. Gross brain injury in the hemisphere ipsilateral to ligation was assessed on a five-point ordinal scale (0-4) as follows: 0=no injury, 1=mild injury with <25% lesion of ipsilateral hemisphere, 2=25-50% lesion, 3=51-75%, and 4=≥75% injury, as previously described Juul, et al., Pediatr Res 2009, 65, 485-492. Area loss was quantified by measuring the area of healthy tissue in the ipsilateral hemisphere normalized to the contralateral hemisphere, according to the following equation:

$$\\ \% Area Loss = ( 1 - \frac{ipsilateral area}{contralateral area} ) \times 100 \%$$

Immunofluorescence and confocal imaging. PLGA-PEG uptake in the brain and microglial morphology was evaluated by placing freshly extracted brains in a formalin-to-30% sucrose gradient and then sectioning on a Leica cryostat into 30 μm sections. For microglia, a primary antibody solution (1:250 rabbit anti-Iba1, Wako) was prepared in 1×PBS containing 1% Triton-X (Sigma) and 3% normal goat serum (Sigma) and was added to tissue sections for 4 h in a humidified chamber at room temperature. Sections were washed twice in 1×PBS. A secondary antibody solution was prepared in 1×PBS and 1% Triton-X and added to tissue sections for 2 h. For neurons, a pre-conjugated antibody solution (1:500 anti-NeuN AlexaFluor 488, Abcam) was prepared in 1×PBS containing 1% Triton-X (Sigma) and added to tissue sections for 6 h in a humidified chamber at room temperature. Sections were washed twice in 1×PBS and then stained with 1:10,000 DAPI for 10 min (Invitrogen). Slides were washed and dried for 30 min in the dark. Mounting medium (Dako, Agilent Technologies, Santa Clara, CA) was added to each slide and a glass coverslip placed on top. Slides were stored at 4° C. until imaged on an A1 confocal microscope (Nikon Instruments) and at 20° C. for long-term storage.

Statistical analysis. All statistical analyses were carried out in GraphPad Prism (GraphPad Software Inc, Version 8.4.0). For analysis of complexation efficiency and nanoparticle catalase loading, the unpaired t test with Welch's correction was used to test for significance. Injury data was summarized as a median with IQR. Total area loss and gross injury scores were compared by the two-tailed Wilcoxon-Mann-Whitney U test. Data with a p value <0.05 were considered statistically significant.

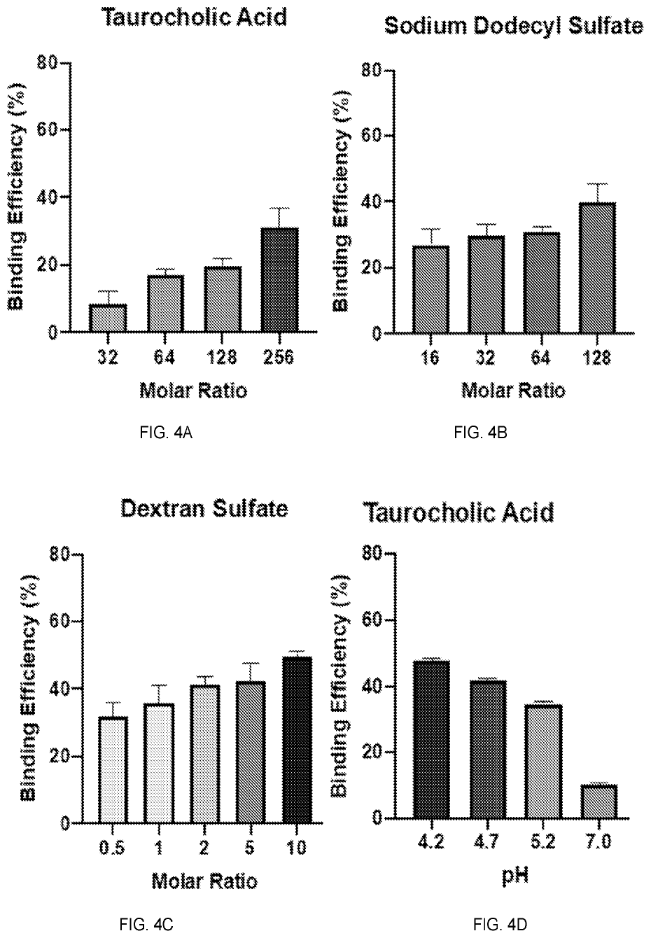

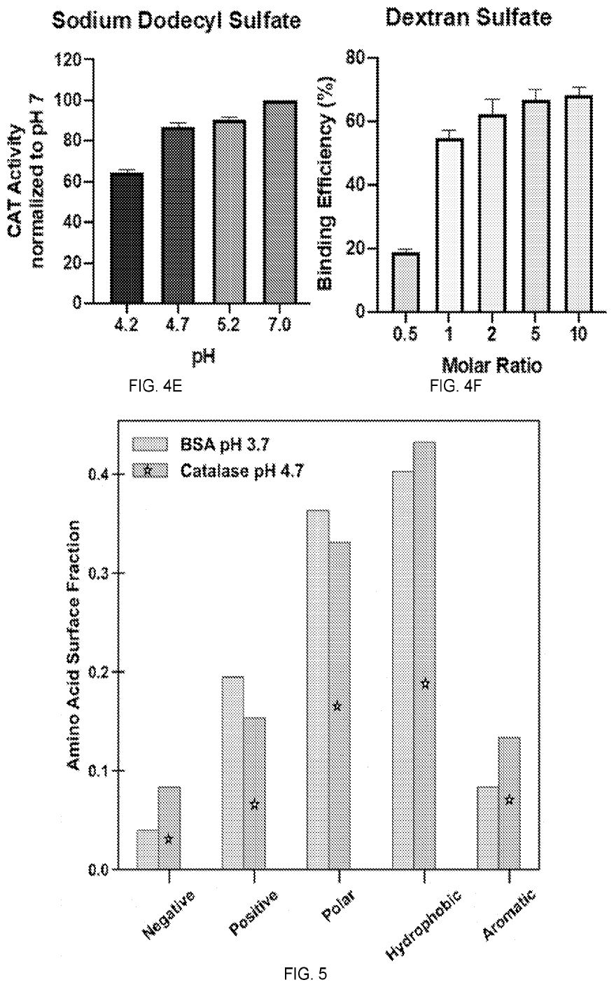

Results. Effect of ion-pairing agent, molar ratio, pH, and buffer ion on complexation efficiency. The HIP complexation of catalase was optimized by investigating 3 common sulfated ion-pairing agents, taurocholic acid (TA), sodium dodecyl sulfate (SDS), and dextran sulfate (DS), across a range of molar ratios. Phosphate buffer at pH 4.7 was used for all initial experiments. For each ion-pairing agent, catalase binding efficiency increased with increasing molar ratio. Catalase incubation with TA at ion-to-protein molar ratios of 32, 64, 128, and 256 resulted in 8.6, 16, 20, and 31% binding efficiencies, respectively (FIG. 4A). For SDS, molar ratios 16, 32, 64, and 128 led to 27, 30, 31, and 40% binding efficiencies (FIG. 4B), and molar ratios of DS at 0.5, 1, 2, 5, and 10 led to 32, 36, 41, 42, and 50% binding efficiencies, respectively (FIG. 4C).

To further optimize complexation, the role of pH on catalase binding efficiency was investigated using DS as the ion-pairing agent. Lower pHs led to greater binding efficiency: at pH 4.2, 4.7, 5.2, and 7.0, binding efficiencies of 48, 42, 35, and 10%, respectively, were observed (FIG. 4D). However, lower pH was also associated with higher catalase degradation, as measured by a loss of activity. When normalized to catalase activity in phosphate buffer at pH 7, catalase at pH 4.2, 4.7, and 5.2 retained 65, 87, and 90% of its activity, respectively (FIG. 4E).

As phosphate has a reduced buffering capacity at acidic pHs, DS-catalase complexation was investigated with citrate buffer, which has a working range of pH 3.0-6.2. At ion-to-protein molar ratios of 0.5, 1, 2, 5, and 10, binding efficiencies of 19, 55, 62, 67, and 68%, respectively, were observed (FIG. 4F). In comparison to phosphate buffer, citrate buffer achieved significantly higher binding efficiencies at every molar ratio above 0.5 (p<0.05 for all).

Molecular scale features of protein-ion complexes. In order to understand any potential differences in the behavior of DS, SDS and TA when binding to proteins, MD simulations were performed to evaluate the structure and dynamics of protein-ion complexes. As a model protein, BSA was used instead of catalase owing to its smaller size, which enables significantly longer simulation times. BSA is well matched to catalase, having a similar profile of surface amino acid residues as shown in FIG. 5. As such, it is reasonable to expect that the nature and extent of protein-ion interactions obtained from the BSA/ion-pairing agent simulations will provide useful insight to the behavior of other proteins with a similar surface profile.

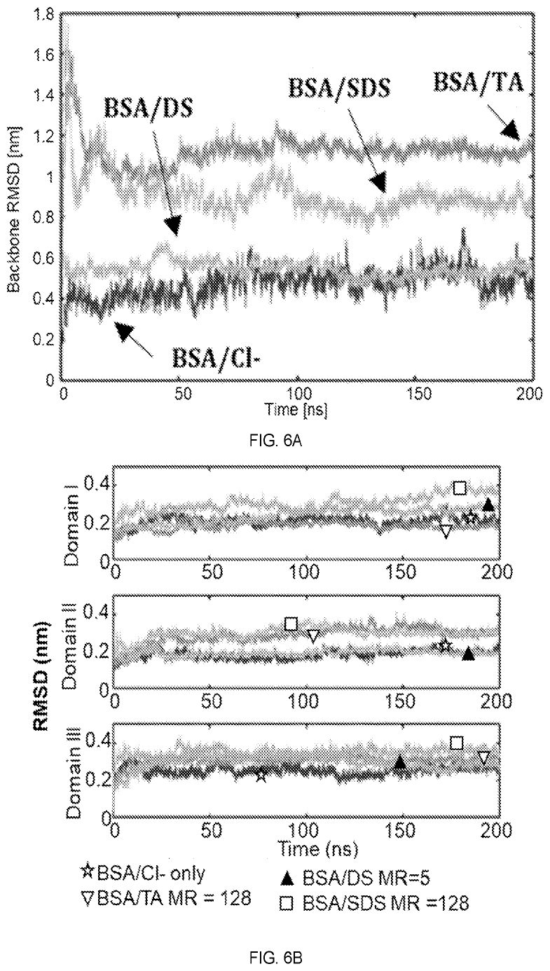

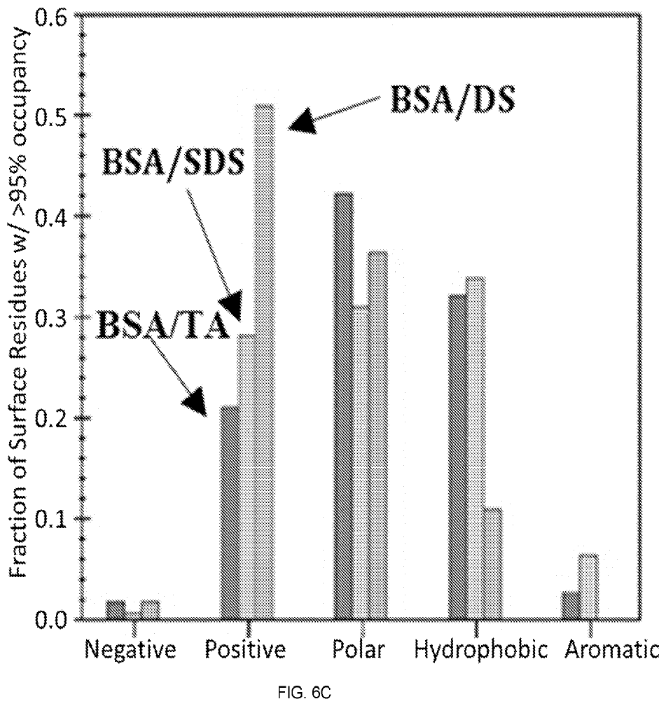

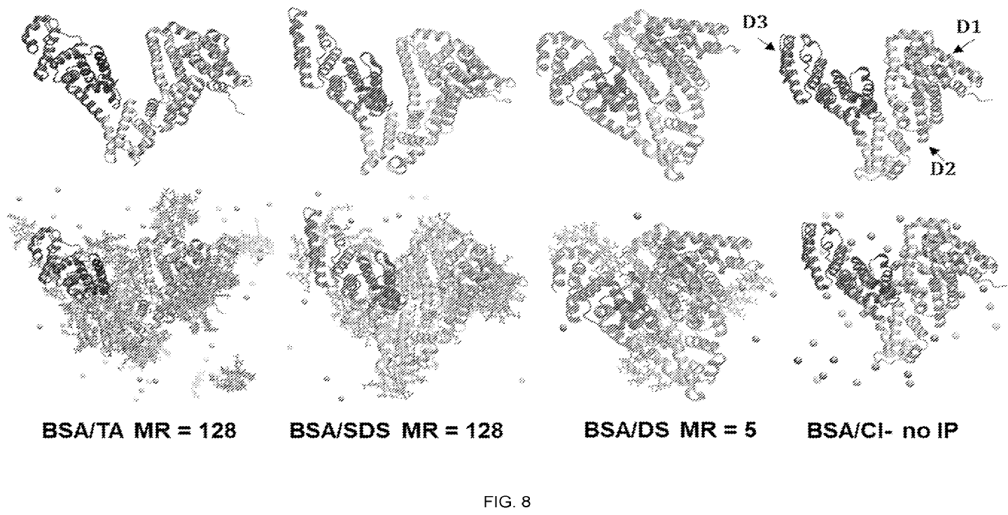

Following completion of the MD simulations, the structure of BSA and related conformational changes was analyzed. The chemical interactions between ions and different types of residues was analyzed at mole ratios of 128 (SDS/TA) and 5 (DS), which were selected to roughly control for a consistent number of anionic sulfate groups between DS (120 total) and SDS/TA (128 total). The MD simulation analyses for these systems are shown in FIGS. 6A-6C. As previously noted by Baler et al. (J Phys Chem B 2014, 118, 921-930), BSA at pH 3.7 was expected to undergo a conformational rearrangement of tertiary structure even on the timescale of MD simulation (usually hundreds of nanoseconds). FIG. 6A shows BSA backbone root mean squared deviation (RMSD) from its crystal structure as a function of simulation time for the three ion-paired systems as well as a control system with only Cl− present for charge neutralization. The level of conformational change in the control and DS system (RMSD 0.5 nm at 200 ns) corresponds well to the expected structure of BSA in the N-isoform. In contrast, BSA conformational changes in the SDS and TA systems (RMSD 0.9 nm and 1.1 nm at 200 ns) indicate that the protein is transitioning from the N to the F-isoform. This suggests that DS complexation is able to retain BSA's native state, unlike SDS and TA complexation. Snapshots of the final structures of each of the simulations are provided in FIG. 8. The individual domains of BSA do not undergo any significant unfolding for any of the systems (FIG. 6B). Finally, SDS and TA behave similarly from the point of view of the dominant chemical interactions on the BSA surface (FIG. 6C), showing significant interactions with hydrophobic and aromatic residues. In contrast, DS has comparatively very few interactions with these residue types.

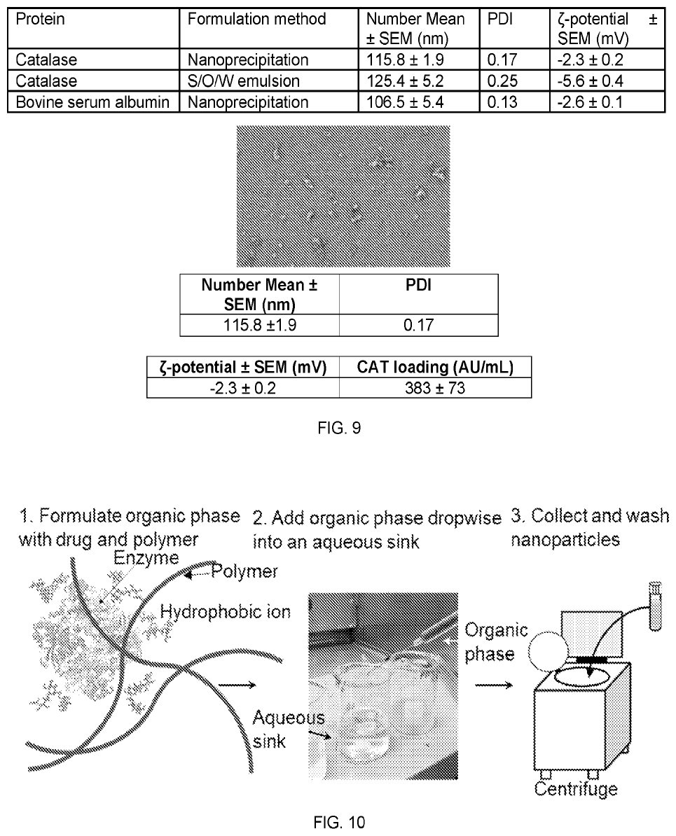

Effect of nanoparticle formulation method on catalase loading and protection. DS-catalase complexes were incorporated into PLGA-PEG nanoparticles using previously published methods for nanoprecipitation and solid/oil/water (S/O/W) emulsion (Patel, et al., J Microencapsul 2014, 31, 542-550; and Yang, et al., Drug Dev Ind Pharm 2009, 35, 959-968). Dynamic light scattering results are summarized in FIG. 9. By nanoprecipitation, catalase-loaded nanoparticles had an average diameter of 115.8 nm, polydispersity index (PDI) of 0.17, and ζ-potential of −2.3 mV. By emulsion, catalase-loaded nanoparticles had an average diameter of 125.4 nm, PDI of 0.25, and ζ-potential of −5.6 mV. FIG. 9 also includes size and surface charge characterization of a blank formulation, used as a control for in vivo studies described in the following section. For these nanoparticles, DS was complexed with BSA at a molar ratio of 5 and pH 3.8.

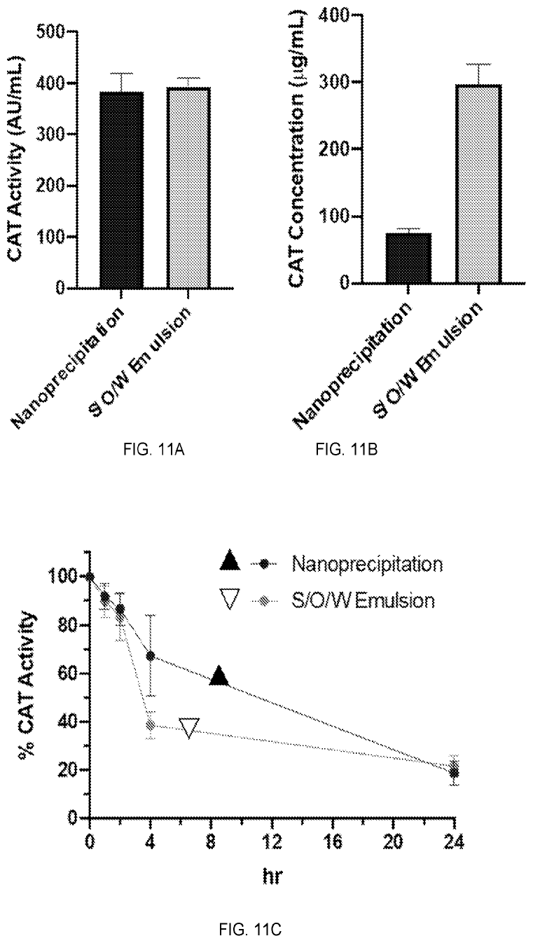

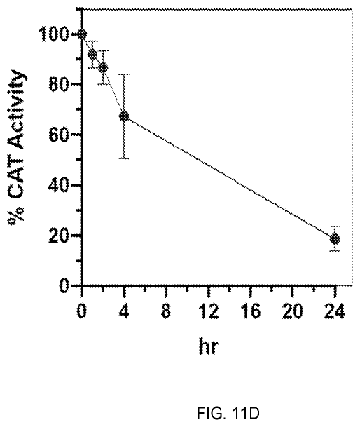

To compare the two catalase nanoparticle formulations, catalase loading was assessed by activity and mass. In terms of catalase activity, both formulations achieved similar loading: nanoprecipitation particles had a mean±standard deviation 383±73 active units (AU) of catalase per mL nanoparticles, while emulsion particles had 393±34 AU/mL (FIG. 11A). In terms of catalase loading by mass, however, nanoprecipitation particles (76±12 μg/mL) had significantly less catalase than emulsion particles (298±59 μg/mL, p=0.0003) (FIG. 11B). Together, these two results indicate higher catalase deactivation by the emulsion process. Catalase activity after nanoprecipitation averages around 5,000±960 AU/mg (as supplied by Sigma); emulsion results in catalase activity of 1320±110 AU/mg. The emulsion process therefore corresponds to 74% (±2%) deactivation of catalase.

Next, nanoparticle protection of catalase was assessed in biological media. To mimic degradative serum conditions in vivo, nanoparticles were incubated in a 0.2% pronase solution and measured catalase activity at 0, 1, 2, 4, and 24 h (FIG. 11C). Activity was normalized to the 0 h timepoint. No significant differences were observed between formulations at each timepoint, and 20% of initial catalase activity was retained by both formulations at the end of the 24 h experimental window.

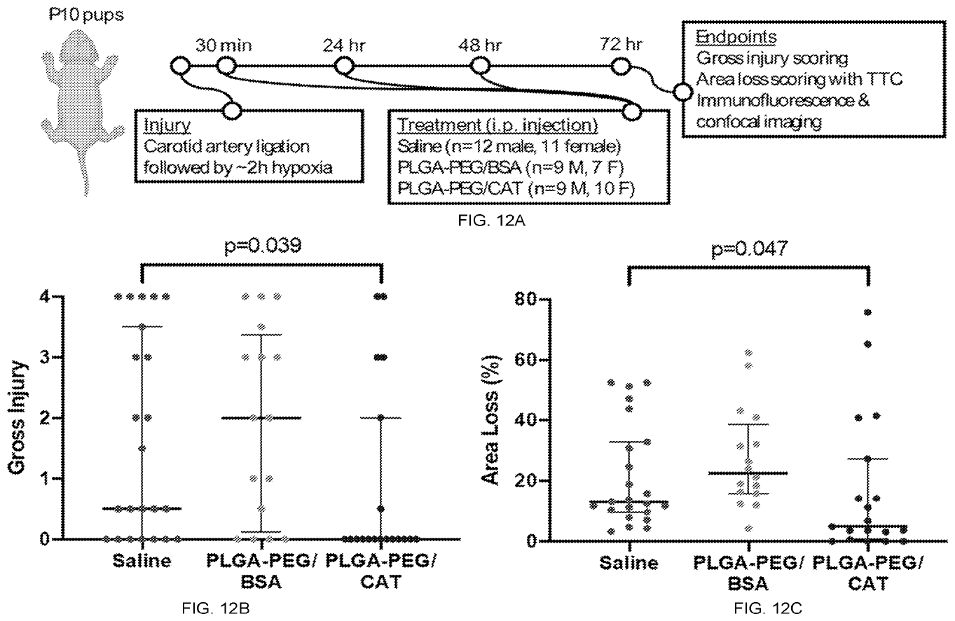

Effect of catalase-loaded nanoparticles on brain injury severity in neonatal rats. Given the significant catalase deactivation by emulsion, nanoparticles were formulated by nanoprecipitation for in vivo investigation. The efficacy of catalase-loaded nanoparticles was assessed in the Vannucci model of HIE with postnatal day 10 (P10) rat pups. Treatments of saline, blank nanoparticles (3300 AU BSA/kg), or catalase nanoparticles (3300 AU catalase/kg) were administered intraperitoneally 30 min, 24 h, and 48 h after injury (FIG. 12A). At the 72 h endpoint, a significant reduction in gross injury scores was found after catalase-loaded nanoparticle treatment where median (interquartile-range, IQR) score was 0 (0-2) compared to saline treatment (0.5, 0-3; p=0.039) (FIG. 12B). Treatment with blank nanoparticles (2, 0-3.5) did not have a significant effect on gross injury.

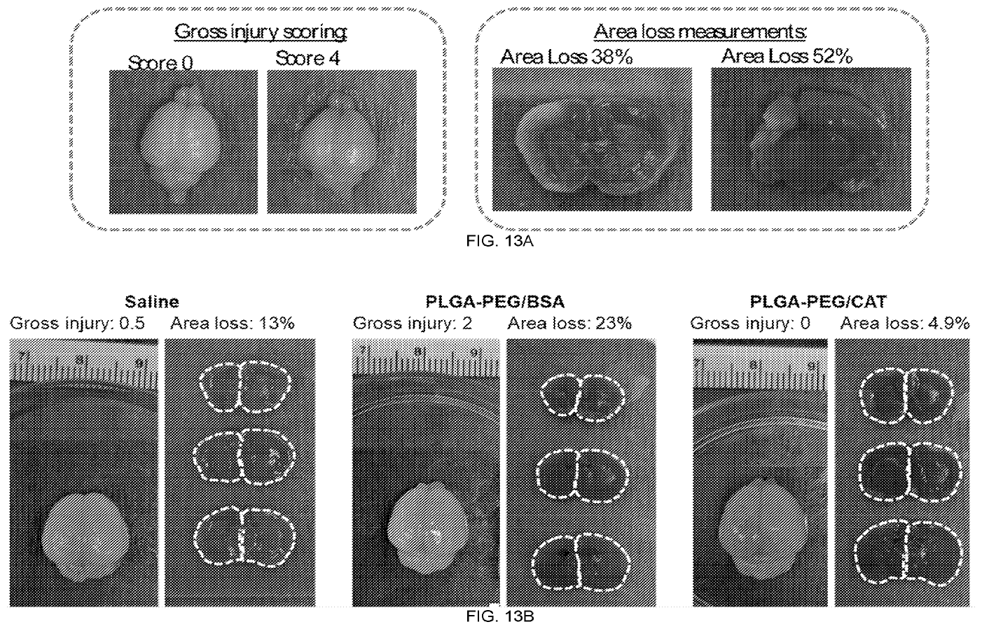

Total area loss measurements supported these results (FIG. 12C). The median (IQR) injury after saline treatment was 13% (10-31%), which was significantly reduced in catalase nanoparticle-treated pups to 4.9% (0.61-27%; p=0.047) but was not significantly affected by treatment with blank nanoparticles (23%, 16-41%). Representative gross injury and area loss images from the median pup in each group are shown in FIG. 13B.

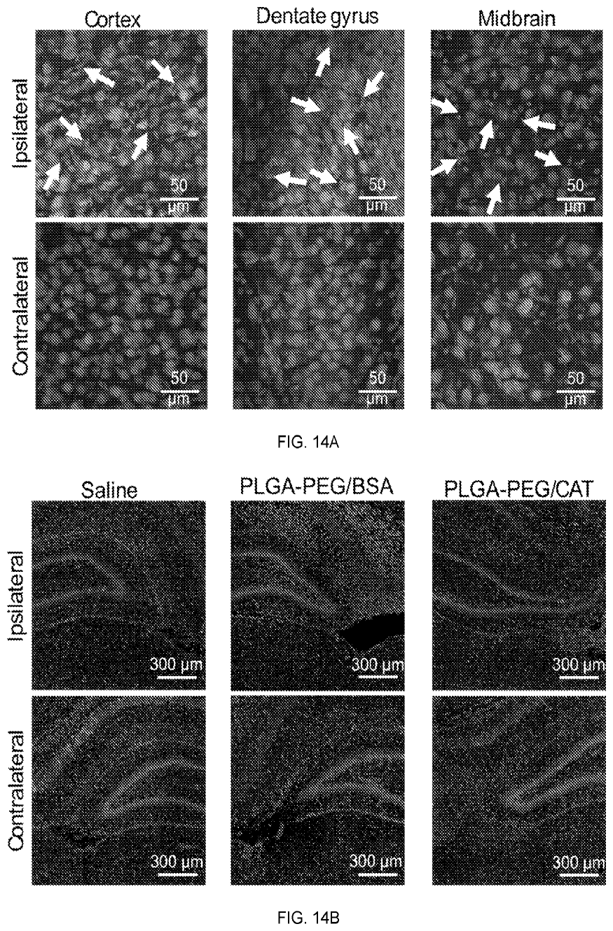

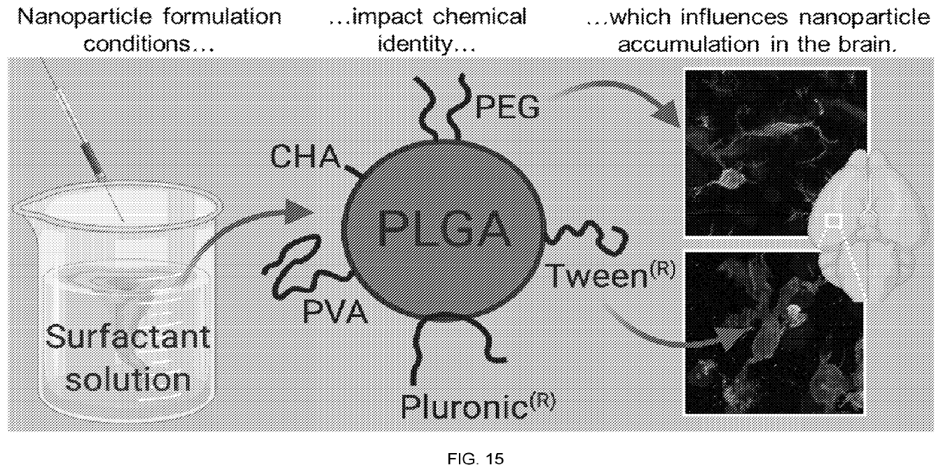

Using confocal imaging, the distribution of nanoparticles in injured brain tissue was confirmed. In the contralateral hemisphere, nanoparticles were observed in blood vessel-like structures, consistent with normal blood-brain barrier function. In contrast, PLGA-PEG nanoparticles were widely distributed through the ipsilateral hemisphere, including the cortex, dentate gyrus, and midbrain regions (FIG. 14A). Imaging of microglia in the hippocampal region demonstrated a cell-level response to PLGA-PEG/CAT nanoparticle treatment. Microglia were higher in number and density in the ipsilateral hemisphere compared to the contralateral hemisphere in both the saline and PLGA-PEG/BSA control groups. After catalase-loaded nanoparticle treatment, microglia number and density appear more consistent between hemispheres, supporting improvement of neuroinflammation (FIG. 14B). An improvement of disease pathology at a cellular level is seen in FIG. 14B. Microglia, the brain's immune cells, increase in number and density after a hypoxic-ischemic insult as part of a strong neuroinflammatory response. Evidence of this response is seen in both control groups, where the ipsilateral hemispheres of Saline and the Blank (PLGA-PEG/BSA) nanoparticle groups have denser green signals compared to their respective contralateral hemispheres. In contrast, both hemispheres of the catalase nanoparticle-treated brain have microglia with similar densities. This result suggests that the strategy to alleviate the oxidative stress burden in the injured brain may feed forward into improving other disease pathways, specifically neuroinflammation.

Conclusion. Neonatal hypoxic-ischemic brain injury often results in a lifelong burden of disease, and strategies to better treat this condition are needed. The antioxidant enzyme catalase is a promising therapeutic which would benefit from PLGA-PEG nanoparticle delivery for improved protection from serum proteases and improved delivery into the brain parenchyma. However, large hydrophilic enzymes do not easily partition into the polymer nanoparticle matrix. Hydrophobic ion pairing could be used to formulate DS-catalase complexes with 68% binding efficacy. Molecular Dynamics (MD) simulations supported that DS binding did not interfere with the native secondary or tertiary protein structure. The DS-catalase complexes were then used to develop catalase-loaded nanoparticles with high catalase activity and protection of enzyme activity for at least 24 h in degradative conditions. Finally, catalase-loaded nanoparticles were found to be significantly neuroprotective in the P10 Vannucci model, resulting in reduced injury scores as well as improved microglial morphology compared to saline and blank nanoparticle controls. Catalase-loaded PLGA-PEG nanoparticles are therefore a promising intervention for further research on the treatment of neonatal brain injury.

This experimental example also shows that catalase can be loaded into PLGA-PEG nanoparticles and that the nanoparticles retained activity for at least 24 hours in degradative conditions. The catalase nanoparticles are neuroprotective of neonatal hypoxic-ischemic encephalopathy and nanoparticle-mediated delivery of catalase may be effective against many diseases of oxidative stress.

Experimental Example 2. Abstract. Surfactants influence polymer nanoparticle fate within the brain. Abstract. Drug delivery to the brain is limited by poor penetration of pharmaceutical agents across the blood-brain barrier (BBB), within the brain parenchyma, and into specific cells of interest. Nanotechnology can overcome these barriers, but its ability to do so is dependent on nanoparticle physicochemical properties including surface chemistry. Surface chemistry can be determined by a number of factors, including by the presence of stabilizing surfactant molecules introduced during the formulation process. Nanoparticles coated with poloxamer 188 (F68), poloxamer 407 (F127), and polysorbate 80 (P80) have demonstrated uptake in BBB endothelial cells and enhanced accumulation within the brain. However, the impact of surfactants on nanoparticle fate, and specifically on brain extracellular diffusion or intracellular targeting, must be better understood to design nanotherapeutics to efficiently overcome drug delivery barriers in the brain. Here, the effect of the biocompatible and commonly used surfactants cholic acid (CHA), F68, F127, P80, and poly(vinyl alcohol) (PVA) were evaluated on poly(lactic-co-glycolic acid)-poly(ethylene glycol) (PLGA-PEG) nanoparticle transport to and within the brain. The inclusion of these surfactant molecules decreases ex vivo microglial uptake and diffusive ability, reflecting the surfactant's role in encouraging cellular interaction at short length scales while reducing nonspecific cellular uptake at longer scales. After in vivo administration, PLGA-PEG/P80 nanoparticles demonstrated enhanced penetration across the BBB and subsequent internalization within neurons and microglia. Surfactants incorporated into the formulation of PLGA-PEG nanoparticles therefore represent an important design parameter for controlling nanoparticle fate within the brain.

Introduction & Summary. The brain's uniquely restrictive biological barriers, including the blood-brain barrier (BBB) and brain parenchyma, make target cells within the brain inaccessible to nearly all therapeutic molecules. While invasive delivery techniques like intraparenchymal injection can increase therapeutic accumulation in the brain, a need remains for noninvasive delivery strategies. Nanotechnology is one promising avenue for systemic delivery of neurotherapeutics since nano-sized particles can overcome transport barriers and achieve accumulation within the brain (Wong, et al., Adv Drug Deliv Rev 2012, 64 (7), 686-700; and Tosi, et al., Expert Opin Drug Deliv 2008, 5 (2), 155-74). Recently, drug-loaded nanoparticles have demonstrated efficacy in models of glioblastoma (Wohlfart, et al., PLoS One 2011, 6 (5), e19121), neurodegenerative disorders (Liu, et al., Bioconjug Chem 2013, 24 (6), 997-1007), and neonatal hypoxic-ischemia (Joseph, et al., Nano Res 2018, 11 (10), 5670-5688), among other brain injuries (Godinho, et al., Mol Pharmaceut 2013, 10 (2), 640-649; and Khalin, et al., Drug Deliv 2016, 23 (9), 3520-3528).

For effective therapeutic delivery in the brain, nanoparticles must be designed to have biologically advantageous properties. One important characteristic is controlled and nontoxic degradability, which impacts drug release kinetics as well as nanoparticle clearance. The biodegradable polymer poly(lactic-co-glycolic acid) (PLGA) is commonly used for nanoparticle formulation as its degradation kinetics are both well-known and tailorable (Park, et al., J Control Release 1994, 30 (2), 161-173; Park, Biomaterials 1995, 16 (15), 1123-1130; and Anderson and Shive, Adv Drug Deliver Rev 2012, 64, 72-82). A second important nanoparticle characteristic is the ability to avoid serum protein binding and subsequent clearance by immune cells. Surface modification with poly(ethylene glycol) (PEG) has been widely used to reduce protein adsorption and limit nonspecific cellular uptake (Owens and Peppas, Int J Pharm 2006, 307 (1), 93-102; and Suk, et al., Adv Drug Deliv Rev 2016, 99 (Pt A), 28-51). As a result, a PEG surface layer can confer enhanced nanoparticle diffusivity, which is especially important in the confined brain extracellular space (ECS) (Nance, et al., Sci Transl Med 2012, 4 (149), 149ra119). Since nanoparticle surfaces mediate interactions between the nanoparticle and biological environment, further surface functionality can be added to PLGA-PEG nanoparticles to improve cell penetration or cell-specific targeting.

One important class of molecules present at nanoparticle surfaces are surface acting agents, referred to as surfactants. During the formulation process of PLGA-PEG nanoparticles, surfactants are commonly introduced to reduce surface tension at the interface between the organic polymer solution and the aqueous phase. Although nanoparticles can be formulated without surfactants (Lepeltier, et al., Adv Drug Deliv Rev 2014, 71, 86-97), these molecules promote nanoparticle formation and stabilization. Additionally, studies have shown that surfactant coatings can alter nanoparticle-cell interactions. Tween surfactants, especially Tween 80 (or polysorbate 80, P80), can specifically enhance nanoparticle accumulation in the brain (Kreuter, et al., J Drug Target 2002, 10 (4), 317-325). Pluronic® surfactants, including poloxamer 188 (Pluronic® F68, F68) and poloxamer 407 (Pluronic® F127, F127), are able to inhibit P-glycoprotein efflux transporters to remain localized intracellularly (Kreuter, J Nanosci Nanotechno 2004, 4 (5), 484-488; Lo, J Control Release 2003, 90 (1), 37-48; and Jain, et al., Acta Biomater 2015, 24, 140-51). However, nearly all prior studies have evaluated surfactant effects after incubating previously-formulated nanoparticles in fresh surfactant solutions to produce a dense surface coating. Moreover, none have investigated nanoparticle diffusion past the BBB, which has dependence on surface presentation of PEG (Xu, et al., Acs Nano 2015, 9 (9), 9217-9227). It was hypothesized that nanoparticles formulated with PEG and surfactants may be optimally designed for transport to and within the brain.

In the present study, PLGA-PEG formulations were compared with surfactants P80, F68, and F127 to a control formulation without surfactant in deionized (DI) water. The surfactants poly(vinyl alcohol) (PVA), the most commonly used surfactant for nanoparticle stabilization (Yang, et al., J Control Release 2014, 192, 202-8), and cholic acid (CHA), an anionic surfactant (Xu, et al., J Control Release 2013, 170 (2), 279-86). were studied. With each formulation, nanoparticle transport was evaluated in the brain at multiple time and length scales, i.e., molecular-level diffusion as well as cellular- and whole organ-scale nanoparticle accumulation. The results provide insight into nanoparticle design for improved penetration of biological barriers for therapeutic delivery in the brain.

Materials and Methods. Materials. PLGA45k (50:50)-mPEG5k (PLGA-PEG) or PLGA45k (50:50) (PLGA) polymers were purchased from Akina PolySciTech. AlexaFluor 555 (AF555) and 647 (AF647) NHS Ester for polymer labeling were purchased from ThermoFisher. Cholic acid (bile salts), Pluronic® F68, Pluronic® F127, P80, and PVA (27 kDa MW) were purchased from Sigma. Solvents for nanoparticle formulation, including acetone and 1× phosphate buffered saline (PBS), were used as received. Capillary depletion buffer (CDB) was made with 10 mM (4-(2-hydroxyethyl)-1-piperazineethanesulfonic acid) (HEPES), 141 mM sodium chloride (NaCl), 4 mM potassium chloride, 2.8 mM calcium chloride, 1 mM magnesium sulfate, 1 mM monosodium phosphate, and 10 mM glucose (Sigma). CDB with dextran (CDB-D) was made by dissolving 2.6 g dextran (67,300 Da MW, Sigma) in 7.4 mL CDB. Slice culture media (SCM) was made with 50% minimum essential media, 45% Hank's Balanced Salt Solution (HBSS) with calcium and magnesium, 5% horse serum, and 1% glutamine and penicillin-streptomycin each (Gibco). FACS media was formulated with 90% 1×HBSS without calcium or magnesium, 10% fetal bovine serum, and 1% 1M HEPES. Percoll solution for FACS was purchased from Sigma.

Nanoparticle formulation. Nanoparticles were prepared by nanoprecipitation. PLGA-PEG or PLGA was dissolved in acetone at a concentration of 20 mg/mL. The polymer solution (organic phase) was then added dropwise into 25 mL of one of the following aqueous solutions: 1% P80, 5% PVA, 1% F127, 5% F68, 3% CHA, or no surfactant in deionized water. Nanoparticles formed spontaneously and were stirred for 3 h at 700 rpm to remove the organic solvent. Nanoparticles were collected and washed twice by ultracentrifugation with deionized water at 100,000×g for 25 min. Finally, the nanoparticles were resuspended in 1 mL deionized water or in sterile PBS for animal experiments. Nanoparticles were used immediately. For fluorescently-labeled nanoparticles, the same nanoparticle formulation procedure was used with PLGA and PLGA-PEG and conjugation of AF555 or AF647 was achieved by attachment to the free COOH on the PLGA backbone, as described previously (Nance, et al., Sci Transl Med 2012, 4 (149), 149ra119).

Nanoparticle characterization by dynamic light scattering and mass spectrometry. The particle size and PDI of PLGA-PEG nanoparticles formulated in CHA, (PLGA-PEG/CHA), F127 (PLGA-PEG/F127), F68 (PLGA-PEG/F68), P80 (PLGA-PEG/P80), PVA (PLGA-PEG/PVA), or no surfactant (PLGA-PEG/DI) and PLGA nanoparticles formulated in F127 (PLGA/F127) were measured by dynamic light scattering. The ζ-potential was determined using a zeta potential analyzer (NanoSizer Zeta Series, Malvern Instruments, Malvern, UK). Samples were diluted to appropriate concentrations to obtain accurate measurements in 10 mM NaCl at room temperature, pH 7.4, as described previously (Xu, et al., J Control Release 2013, 170 (2), 279-86).

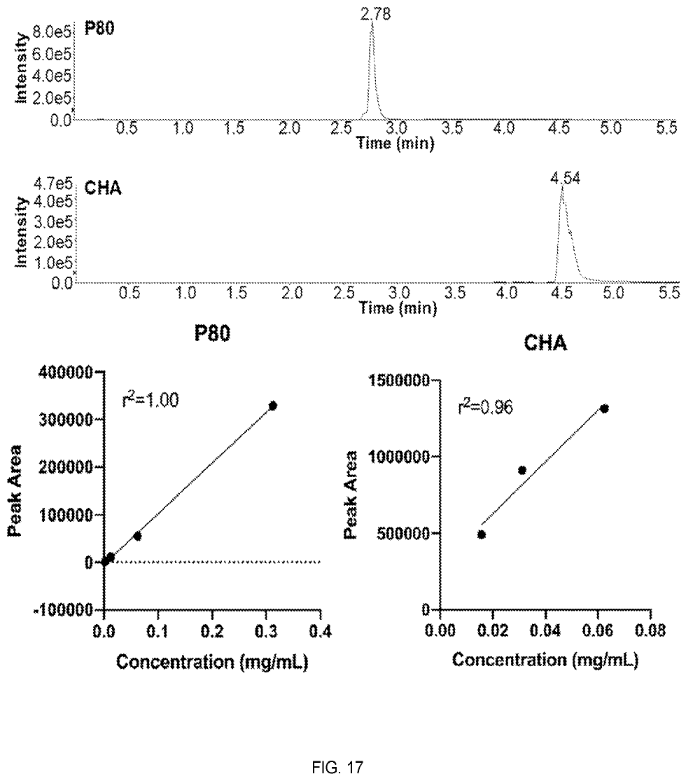

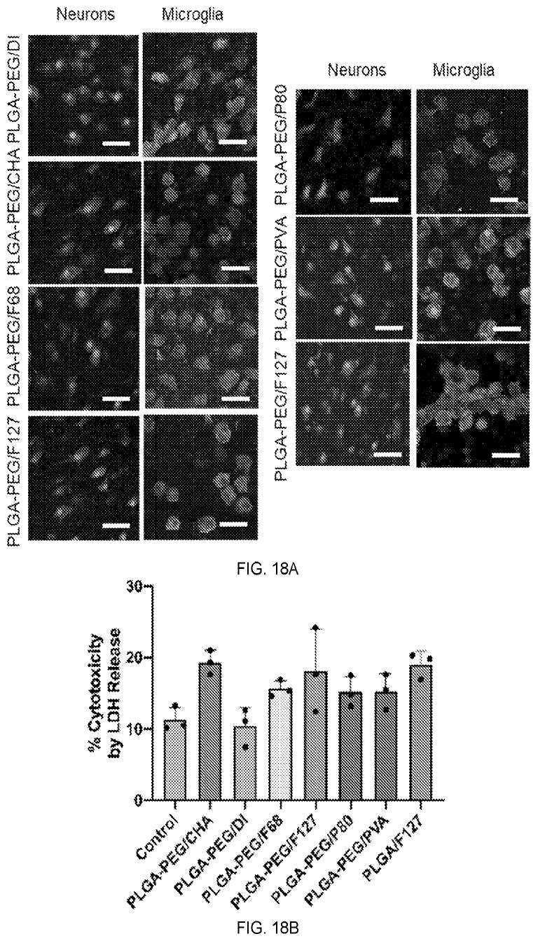

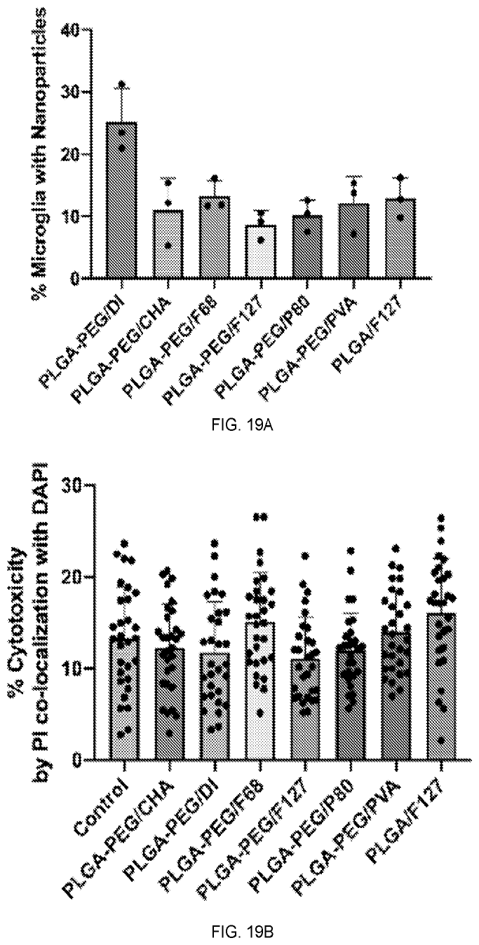

To quantify the amount of surfactant present in each formulation, standard solutions of each surfactant were injected into a triple quadrupole LC-MS/MS system (AB Sciex 5600 QTOF) equipped with a Waters BEH column (50 mm, 2.1×150 mm). Surfactant was eluted using two mobile phases, HPLC grade water and acetonitrile at 0.3 mL/min and identified on the chromatogram by molecular weight. For surfactants P80 and CHA, where a sample peak was identified, a calibration curve from standard solutions was created and used to determine the amount of surfactant present in PLGA-PEG/P80 and PLGA-PEG/CHA, respectively (FIG. 17A). For surfactants F68, F127, and PVA, no sample peak could be identified (FIG. 17B).

Animal experiments and ethics statement. This study was performed in strict accordance with the recommendations in the Guide for the Care and Use of Laboratory Animals of the National Institutes of Health. All of the animals were handled according to approved Institutional Animal Care and Use Committee (IACUC) protocols (#4383-01 and #4383-02) associated with approved Animal Welfare Assurance (#A3464-01) on file with the NIH Office of Laboratory Animal Welfare (OLAW), registered with the United States Department of Agriculture (USDA, certificate #91-R-0001), and accredited by AAALAC International. Every effort was made to minimize suffering. Time-mated pregnant female Sprague-Dawley rats (virus antibody-free CD® (SD) IGS, Charles River Laboratories, Raleigh, NC, USA) were purchased and arrived on estrous (E) day 17. Dams were housed individually and allowed to acclimate to their environment for a minimum of 3 days prior to delivering. The day of birth was defined as postnatal (P) day 0. Litters containing both sexes were cross-fostered and culled to 12 animals early after birth. Before and after the experiment, each dam and her pups were housed under standard conditions with an automatic 12 h light/dark cycle, a temperature range of 20-26° C., and access to standard chow and autoclaved tap water ad libitum. The pups were checked for health daily.

Cell uptake and death in organotypic brain slices. In cultured brain slices, cytotoxicity of surfactant-formulated nanoparticles was determined by lactate dehydrogenase (LDH) assay and propidium iodide (PI) staining. Brain slices were collected from P9 pups and left to rest overnight in the incubator (maintained at 37° C., with constant humidity and 95% air and 5% CO₂) (Liao, et al., J Biol Eng 2020, 14, 3). Each nanoparticle formulation was diluted to 1 mg/mL in SCM and 100 μL of the diluted nanoparticles was added on top of each slice. The media was collected 4 h after treatment start time for LDH analysis, and fresh media containing 5 μg/ml PI was added for 1 hour. The slices were then fixed, stained with DAPI, and imaged using a Nikon A1R with a 40× objective. For every slice, five images were acquired from each brain region of interest (cortex and thalamus). Image acquisition settings were consistent for all images. For each image, DAPI+ cells (total cells) and PI+ cells (dead cells) were counted manually in ImageJ (NIH) after applying an Otsu threshold and fluorescent cutoff to aid in visualization. The PI+/DAPI+ cell ratio was expressed as the percentage of dead cells in an individual image.

For LDH cytotoxicity analysis, media samples were thawed to room temperature and LDH assays (Cayman Chemical) were conducted according to the manufacturer's protocol. 100 μL of the sample was added to 100 μL of LDH reaction buffer in triplicate to 96-well plates on ice and the plates were gently shaken in a 37° C. incubator. After 30 min, the plates were returned to the ice and then measured by UV-Vis (SpectraMax M5, Molecular Devices) for absorbance at 490 nm. Percent cytotoxicity was calculated as the sample absorbance normalized to the 4 h absorbance of the Triton-X condition×100%.

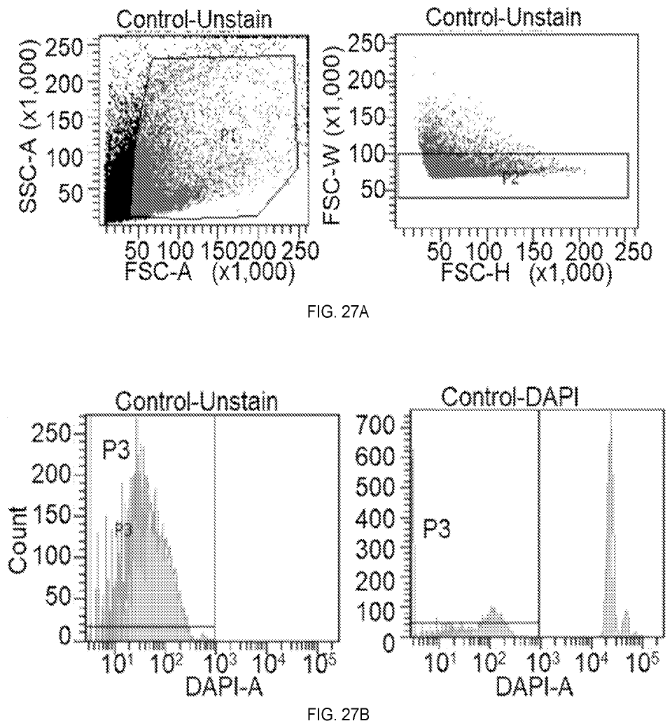

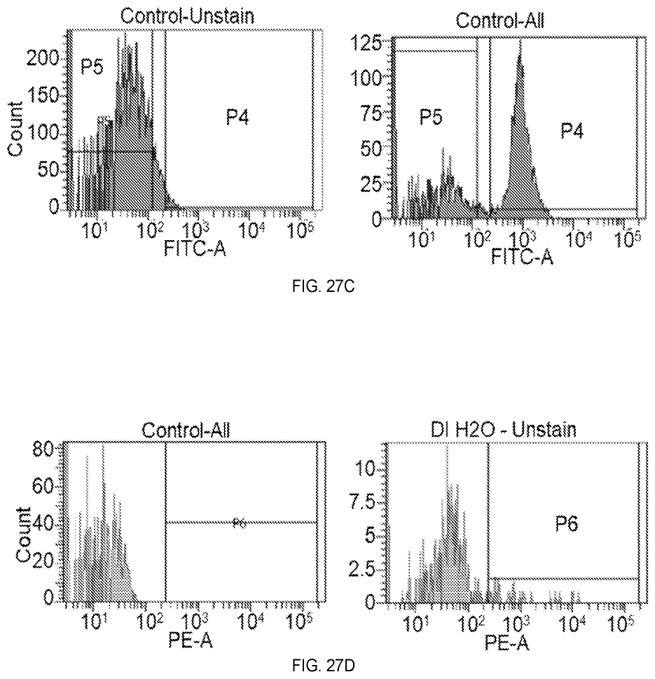

Flow cytometry. Flow cytometry was used to quantify the proportion of microglia with nanoparticle uptake. Fresh brain slices (3 slices per sample and n=3 samples per condition) were prepared as described previously and immediately incubated with a nanoparticle condition: 100 μL of PLGA-PEG/DI, PLGA-PEG/CHA, PLGA-PEG/F68, PLGA-PEG/F127, PLGA-PEG/P80, PLGA-PEG/PVA, or PLGA/F127, or no nanoparticles. After 4 h, slices from each experimental group were placed in 1 mL Accutase. Samples were gently shaken on ice for 30 min and then carefully pipetted to ensure tissue was fully homogenized. The sample was then transferred to a new tube through a top filter (Pierce Tissue Strainers) until all the homogenate was filtered. During this process, HBSS and 25 mM HEPES were added to dilute homogenate to a final volume of 10 mL. The tube was spun down at 600×g at 4° C. for 5 min to pellet cells, then aspirated and the supernatant discarded. 100% FBS was then added to resuspend the cell pellet. Percoll Solution (final concentration 33%, GE Healthcare) was added to the cell suspension and mixed well, and then FACS media was added to the suspension. The cell suspension was centrifuged for 15 min at 800×g and 4° C., and then the supernatant was aspirated, leaving the cell pellet at the bottom. To wash excess Percoll Solution, the pellet was resuspended in FACS media and centrifuged for 10 min at 600×g and 4° C., and again the supernatant was removed. The final pellet was resuspended in FACS media for staining. Fc block (BD Biosciences) was added to the FACS media cell suspension and incubated for 5 min on ice, and then cells were stained with DAPI (1:10,000) and FITC CD11b (1:200). Appropriate controls for CD11b gating were performed with an aliquot of the control sample. The cells were stained with the above stains for 15 min and washed 3 times with FACS media for 4 min at 1000×g and 4° C. The BD LSRII (BD Biosciences) machine recorded cells in each sample with fluorescence in the DAPI, CD11b, and AF555 channels until 100,000 events (live cells) were reached. Analysis of the cytometry data was performed in FCS Express 7 Research and representative data is shown in FIGS. 27A-27D.

Biodistribution, capillary depletion, and nanoparticle quantification. AF647-labeled PLGA and PLGA-PEG nanoparticles were administered via tail vein (150 mg/kg) in P9 pups (n=5). After 4 h, the pup was sacrificed and the animal was perfused with 20 mL 1×PBS. Capillary depletion was conducted on freshly extracted brains (n=4) according to the protocol described by Banks et al. (J J Virol 2001, 75 (10), 4681-91). Briefly, brains were homogenized in 0.8 mL CDB and then mixed with 1.6 mL CDB-D on ice. The homogenate was centrifuged at 5400×g for 15 min at 4° C. The middle, clear layer was separated as the capillary-depleted brain fraction and the bottom, red pellet was resuspended in 0.3 mL PBS as the capillary-rich brain fraction. All other organs were homogenized in PBS at a 1 g/mL concentration and centrifuged at 10,000×g for 10 min at 4° C. to remove cellular debris.

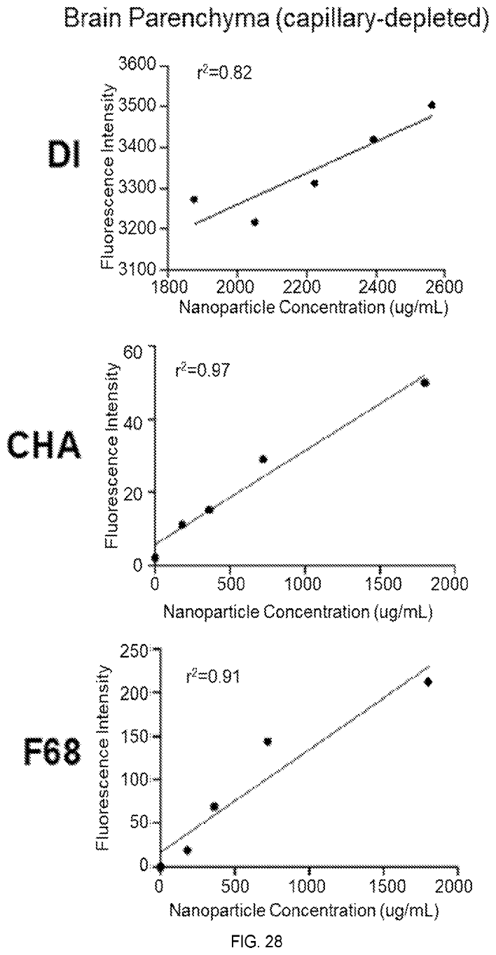

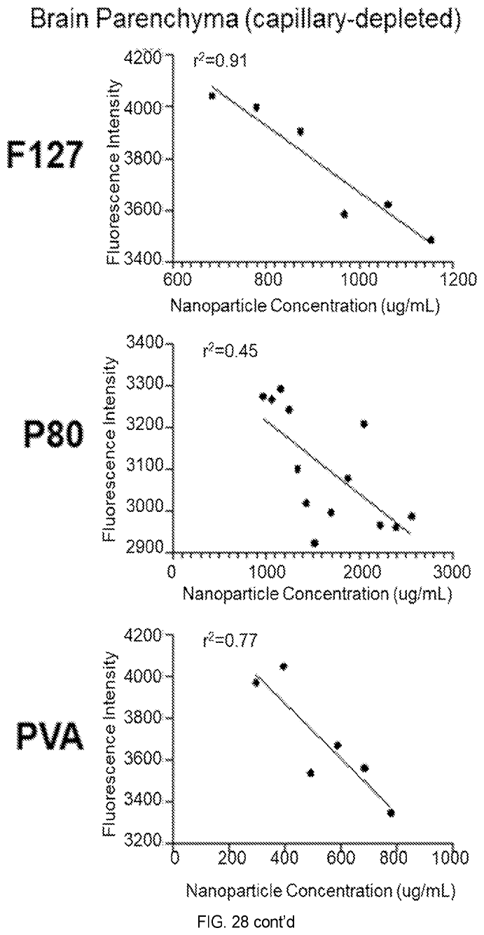

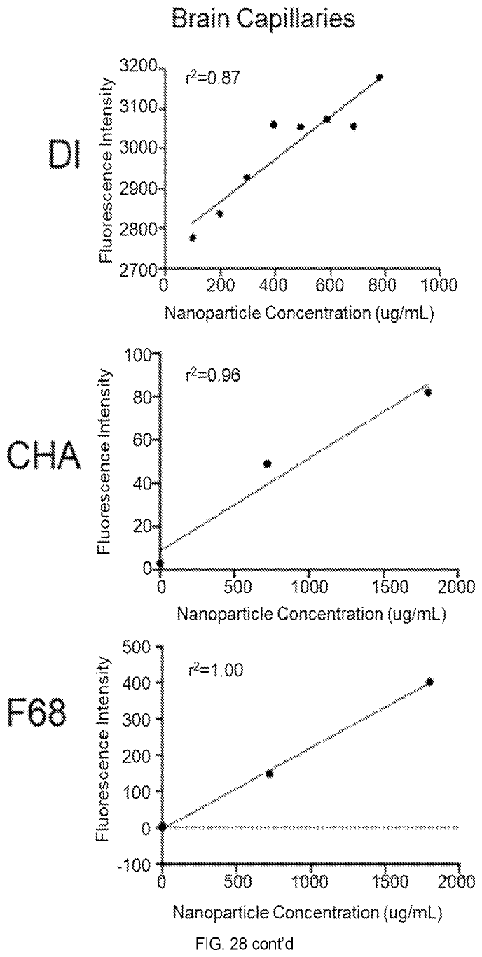

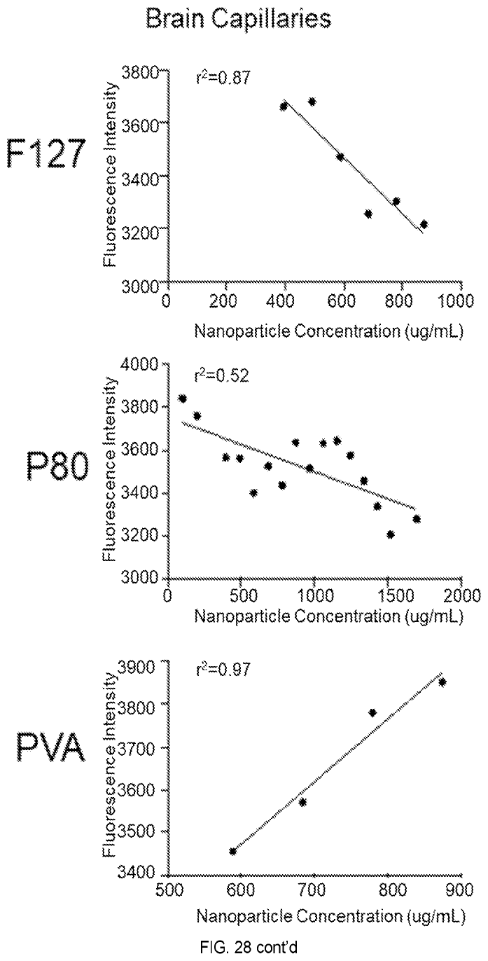

Nanoparticle concentration in the capillary-rich and capillary-depleted brain fractions and major organs were determined by measurement of sample fluorescence intensities (excitation 625 nm/emission 665 nm) using UV-Vis spectroscopy. Separate calibration curves were created for each tissue fraction and nanoparticle formulation combination (FIG. 28). The same nanoparticle batch was used for injection and calibration curves. The analysis was conducted by first subtracting blank fluorescence values for tissue from a control animal. All calculated nanoparticle concentrations were normalized by injected dose (ID) and then weight to find % ID per mg tissue.

Immunohistochemistry for microglial and neuronal co-localization. Nanoparticle co-localization after in vivo administration was evaluated for each formulation group by placing freshly extracted brains in a formalin-to-30% sucrose gradient and then sectioning on a Leica cryostat into 30 μm sections. For microglia, a primary antibody solution (1:250 rabbit anti-Iba1, Wako) was prepared in 1×PBS containing 1% Triton-X (Sigma) and 3% normal goat serum (Sigma) and was added to tissue sections for 4 h in a humidified chamber at room temperature. Sections were washed twice in 1×PBS. A secondary antibody solution was prepared in 1×PBS and 1% Triton-X and added to tissue sections for 2 h. For neurons, a pre-conjugated antibody solution (1:500 anti-NeuN AlexaFluor 488, Abcam) was prepared in 1×PBS containing 1% Triton-X (Sigma) and added to tissue sections for 6 h in a humidified chamber at room temperature. Sections were washed twice in 1×PBS and then stained with 1:10,000 DAPI for 10 min (Invitrogen). Slides were washed and dried for 30 min in the dark. Mounting medium (Dako, Agilent Technologies, Santa Clara, CA) was added to each slide and a glass coverslip placed on top. Slides were stored at 4° C. until imaged on an A1 confocal microscope (Nikon Instruments) and at 20° C. for long-term storage.

Cellular co-localization was also evaluated in ex vivo brain tissue. Brain slices were collected from P9 pups and 100 μL of surfactant-formulated PLGA or PLGA-PEG nanoparticles (1 mg/mL) was immediately added on top of each slice. After 4 h, slices were washed with 1×PBS, fixed with 10% formalin for 1 h, and then washed again. For microglial staining, a primary antibody solution (1:250 rabbit anti-Iba1, Wako) was prepared in 1×PBS containing 3% Triton-X (Sigma) and 6% normal goat serum (Sigma). For neuronal staining, the antibody solution (1:500 anti-NeuN Alexa Fluor 488, Abcam) was prepared in 1×PBS containing 3% Triton-X. Antibody solutions were added to the slices for 6 h at room temperature and then washed twice. For microglia, a secondary antibody solution (1:500 goat anti-rabbit Alexa Fluor 488, Abcam) was prepared in 1×PBS containing 3% Triton-X and subsequently was added to the slices at room temperature for 2 h. After washing twice, all slices were stained with 1:10,000 DAPI (Invitrogen) for 15 min and stored in 1×PBS at 4° C. until imaged on an A1 confocal microscope.

Multiple Particle Tracking (MPT) in organotypic brain slices. Fresh brain slices from P9 pups were prepared as described previously (Nance, et al., J Control Release 2014, 189, 123-132; and Nance, et al., J Control Release 2015, 214, 112-20) and used for MPT analysis to evaluate diffusive ability of surfactant-formulated PLGA-PEG nanoparticles in the living brain. Slices were transferred to 35 mm glass bottom imaging disks and 2 μL of AF555-labeled nanoparticles were injected directly into brain tissue. Visualization of the nanoparticles was accomplished with the excitation/emission spectra specific to AF555. Five 6.5 s videos were collected per slice at 10 Hz and 40× magnification via fluorescent microscopy with a cMOS camera (Hamamatsu Photonics Corporation, Bridgewater, NJ). Trajectories from each video were segmented and recorded with respect to x-position (x), y-position (y), and time step (t) using the TrackMate ImageJ plugin.

Selected trajectory features, described below, were extracted.⁴¹ First, geometrically-averaged precision-weighted MSDs were calculated for each trajectory and timestep using the equation:

$$\\ < {r}^{{}_{} 2} >= \frac{1}{N - 1} {\sum }_{i = 0}^{N - n - 1} {\vert {x}_{i + N} - {x}_{i} \vert}^{2}$$

where r² indicates the MSD determined at each step, n, for a total number of steps, N, with 3D position coordinates x(x,y,t).

Then, diffusion coefficients, D, and the anomalous diffusion coefficient (D_{eff}), alpha, were determined by fitting MSD curves to the function:

$$\\ < {r}^{{}_{} 2} ( n ) >= 4 {D ( n Δ t )}^{α}$$

Alpha values of 1 indicate normal diffusive behavior, while values below 1 indicate subdiffusion and values above 1 indicate superdiffusion.

The MSD ratio, which characterizes the shape of the MSD curve, is defined by:

$$\\ < {r}^{{}_{} 2} {>}_{{n}_{1} , {n}_{2}} = \frac{< {r}^{{}_{} 2} ( {n}_{1} ) >}{< {r}^{{}_{} 2} ( {n}_{2} ) >} - \frac{{n}_{1}}{{n}_{2}}$$

where n₁ and n₂ represent the first and last frames of the trajectory, respectively. Ratios below 0 indicate restricted diffusion (subdiffusion) while ratios above 0 indicate superdiffusion.

Trajectory efficiency (E), a measure of the nanoparticle's net displacement compared to the sum of its step lengths, was calculated by the equation:

$$\\ E = \frac{{\vert {x}_{N + 1} - {x}_{0} \vert}^{2}}{{\sum }_{i = 1}^{N - 1} {\vert {x}_{i} - {x}_{i - 1} \vert}^{2}}$$

The fractal path dimension (D_{f}), which can distinguish between confined and random walk trajectories, was calculated from:

$$\\ {D}_{f} = \frac{log ( N )}{log ( {NdL}^{- 1} )}$$

where d is the largest distance between any two positions and L is the sum of all step lengths. Fractal dimension values of 2 indicate random walk trajectories and values above 2 indicate confined diffusion.

Finally, trappedness (pₜ), the probability that a particle with diffusion coefficient D and traced for a period of time NΔt is trapped into a region r₀, is given by:

$$\\ {p}_{t} = 1 - exp ( 0.2048 - 0.25117 ( \frac{DN Δ t}{{r}_{0}^{{}_{} 2}} )$$

All calculations were performed in Python using a package available on GitHub (Curtis, et al., J Open Source Softw 2019, 4 (36)).

Time-of-Flight secondary ion mass spectrometry. ToF-SIMS spectra were acquired on an IONTOF ToF-SIMS 5 spectrometer using a 25 keV Bi₃⁺ cluster ion source in the pulsed mode. Spectra were acquired for both positive and negative secondary ions over a mass range of m/z=0 to 800. The ion source was operated at a current of 0.2 pA. Secondary ions of a given polarity were extracted and detected using a reflectron time-of-flight mass analyzer. Spectra were acquired using an analysis area of 100 micron×100 micron. Positive ion spectra were calibrated using the CH₃⁺, C₂H₃⁺, and C₃H₅ peaks. The negative ion spectra were calibrated using the CH⁻, OH⁻, C₂H⁻, and C₄H⁻ peaks. Calibration errors were kept below 25 ppm. Mass resolution (m/Δm) for a typical spectrum was between 5000 to 5600 for m/z=27 (pos) and between 4000 to 6500 for m/z=25 (neg). PLA-PEG/DI nanoparticle, surfactant and PLA-PEG/surfactant samples were drop cast on cleaned silicon wafers. 5 positive and 5 negative ion spectra were collected from random positions on each sample. Sample preparation and data acquisition was repeated on two separate dates for a total of 10 positive and 10 negative ion spectra per sample type. The positive and negative ion data were analyzed separately to generate a peak list across all spots on all samples. The peak area tables were imported into the NBToolbox spectragui (available online at nb.uw.edu/mvsa/nbtoolbox) and used to create a peak ratio A/(A+B) where A=sum of all P80 peaks and B=sum of all nanoparticle peaks.

Plasma protein adsorption study. Plasma was collected from P9 rat pups by collecting blood into a heparin-coated tube and then centrifuging out cells at 2000×g for 10 min. 100 μL of each PLGA and PLGA-PEG nanoparticles were mixed well with 900 μL plasma and then left in a 37° C. incubator. 4 h later, nanoparticles were pelleted by centrifugation at 100,000×g for 25 min to remove non-adsorbed protein. A small volume of the resuspended nanoparticles was used for dynamic light scattering characterization, as described previously. The samples were also tested for protein concentration with the Pierce BCA Protein Assay Kit (ThermoFisher). Following the manufacturer's instructions, 25 μL of each sample was added to a 96-well plate in triplicate on ice. After addition of 200 μL BCA assay working reagent (50:1 reagent A:B), the plate was gently shaken at 37° C. for 30 min. After 30 min, absorbance of each well was measured at 562 nm on a SpectraMax M5 UV-Vis Spectrophotometer (Molecular Devices).

Statistical analysis. Statistical analysis of in vivo biodistribution and FACS microglial uptake results were made using an unpaired t-test, assuming normality. Statistical analysis of MPT data distributions was conducted using a two-tailed Mann-Whitney U-test. All analysis was performed using GraphPad version 7 (Prism, San Diego, California). A P-value <0.05 was considered statistically significant.

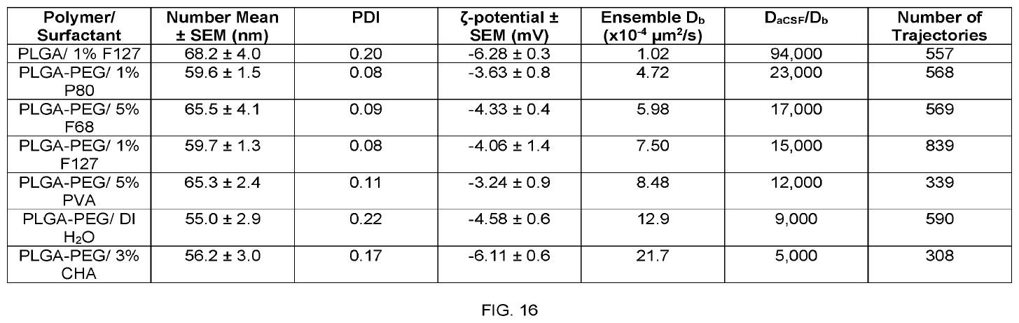

Results. Surfactants and PEG enhance nanoparticle stability and diffusive ability. Nanoparticle size and surface charge were controlled to isolate the role of surfactant on biodistribution, cellular uptake, and diffusive ability. Based on previous work, colloidally stable nanoparticles with hydrodynamic diameters below 114 nm and near-neutral zeta potential (ζ-potential) can transport efficiently to and within the brain (Nance, et al., Sci Transl Med 2012, 4 (149), 149ra119; and Curtis, et al., Colloids Surf B Biointerfaces 2018, 170, 673-682). The formulations used in this study had average diameters between 55-69 nm and ζ-potentials between −6.5 and −3.0 mV (FIG. 16). The PEG layer and surfactant molecules decrease nanoparticle aggregation and increase stability, which is indicated by low polydispersity indices (PDI<0.20) of formulations with both PEG and surfactant. However, nanoparticles without PEG (PLGA/F127) or surfactant (PLGA-PEG/DI) had elevated PDIs of 0.20 and 0.22, respectively. Using mass spectrometry, it was confirmed that surfactant molecules compose a low weight percent of the PLGA-PEG/P80 (0.01%) and PLGA-PEG/CHA (0.005%) formulations (FIG. 17).

To probe molecular-scale interactions in the brain microenvironment, the diffusive ability of each nanoparticle formulation was characterized in 300 μm brain slices prepared from healthy rats. More extensive analysis of nanoparticle diffusion is presented in the following sections, but ensemble-averaged effective diffusion coefficients (D_{b}), its fold-change compared to theoretical nanoparticle diffusion in artificial cerebrospinal fluid (D_{aCSF}), and the number of analyzed trajectories are included as summary statistics in FIG. 16. The results indicate rapid transport of nanoparticles with anionic surfactant molecules (PLGA-PEG/CHA, D_{b}=21.7×10−4 μm²/s) or without surfactant (PLGA-PEG/DI, D_{b}=12.9×10⁻⁴ μm²/s). Diffusion of non-PEGylated nanoparticles with surfactant (PLGA/F127, D_{b}=1.02×10⁻⁴ μm²/s) is significantly hindered, in alignment with previous studies on the importance of a PEG layer for enhanced diffusive behavior (Nance, et al., J Control Release 2014, 189, 123-132).

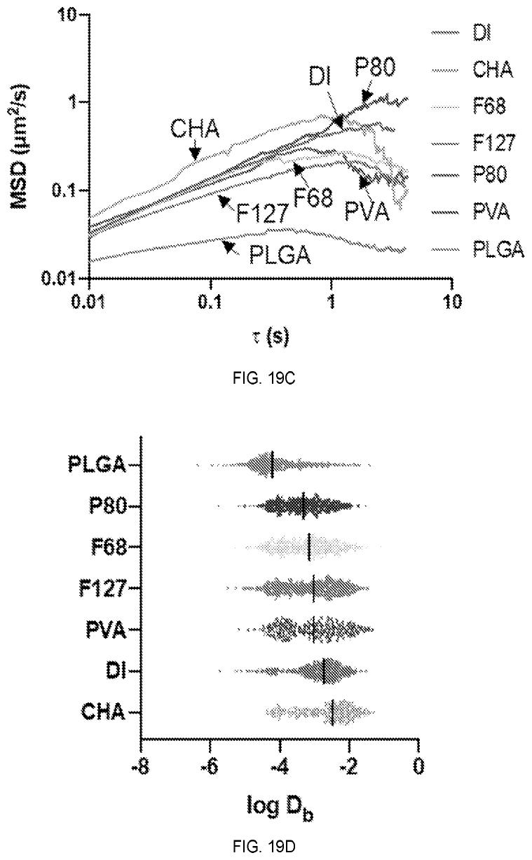

Nonionic surfactants result in decreased cellular uptake and diffusivity ex vivo. Organotypic brain slices enable high-throughput assessment of nanoparticle behavior in the brain, as slices retain multicellular complexity and 3D architecture without in vivo barriers which limit nanoparticle penetration to the brain (Joseph, et al., Bioeng Transl Med 2020, 5 (3), e10175). Nanoparticle uptake into neurons and microglia was first assessed within 4 h of exposure by confocal imaging (FIG. 18A) and quantitation by flow cytometry of microglia (FIG. 19A). The results showed uptake of PLGA-PEG/DI nanoparticles in 25.2% of microglia, and significantly reduced internalization rates 11.0%, 13.2%, 8.6%, 10.1%, 12.1%, and 12.9% for PLGA-PEG/CHA, F68, F127, P80, PVA, and PLGA/F127, respectively. No significant differences exist between nanoparticles formulated with surfactant. Cytotoxicity was investigated as a potential confounding factor in nanoparticle uptake studies, since cells exposed to a high surfactant dose would undergo cell death (Hering, et al., Environ Toxicol Pharmacol 2020, 76, 103353) and therefore not be counted in flow cytometry analysis. Using propidium iodide staining (FIG. 19B) and lactate dehydrogenase release (FIG. 18B) as two measures of cell death after four hours of nanoparticle incubation, all samples were within a normal 5-25% cytotoxicity range.

Each nanoparticle formulation was applied to organotypic brain slices and subsequently analyzed several hundred nanoparticle trajectories to extract diffusion information. At shorter length and time scales, nanoparticle transport is governed by diffusion. Each PLGA-PEG formulation demonstrates a positive slope of ensemble-averaged mean squared displacement (<MSD>) over time, while the PLGA/F127 formulation shows more stagnant growth (FIG. 19C), indicating limited diffusive ability. At a time interval of 0.8 seconds, D_{b} were extracted for each trajectory (FIG. 19D). The ensemble-averaged D_{b} for each formulation at this time scale are available in FIG. 16. The D_{b} distributions indicate significantly enhanced diffusive ability of PLGA-PEG nanoparticles with the anionic surfactant CHA compared to no surfactant (p<0.0001). However, the presence of nonionic surfactants reduced diffusive ability: compared to PLGA-PEG/DI nanoparticles, 1.52-fold, 1.72-fold, 2.16-fold, and 2.73-fold slower diffusion was observed with the PLGA-PEG/PVA, PLGA-PEG/F127, PLGA-PEG/F68, and PLGA-PEG/P80 formulations, respectively. Although the reductions were statistically significant (p<0.001 for all), the effect of surfactant incorporation was much smaller than the effect of the PEG layer. Without a PEG layer, nanoparticles appeared immobilized in the brain: PLGA-PEG/F127 nanoparticles exhibited 7-fold increased diffusive ability compared to PLGA/F127 nanoparticles (p<0.0001).

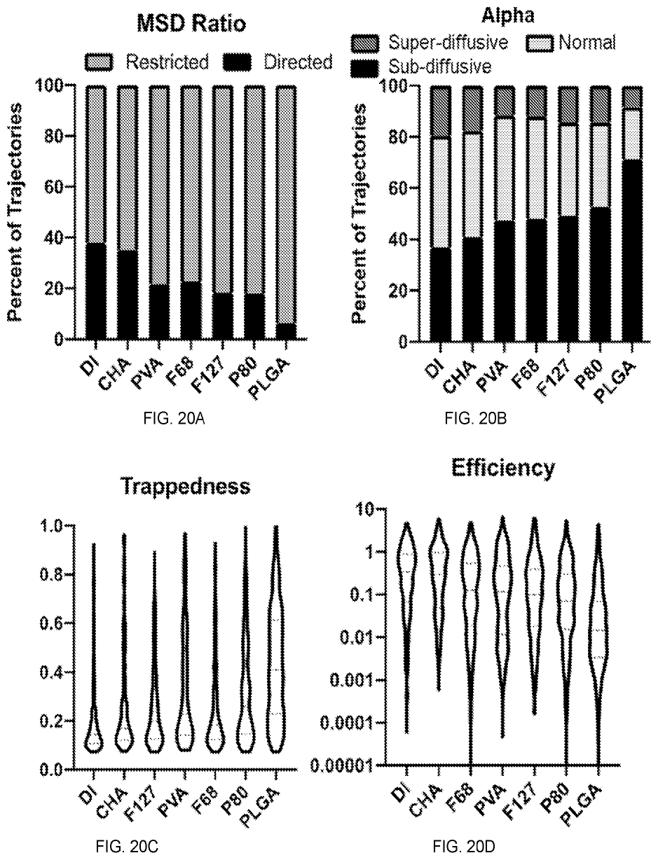

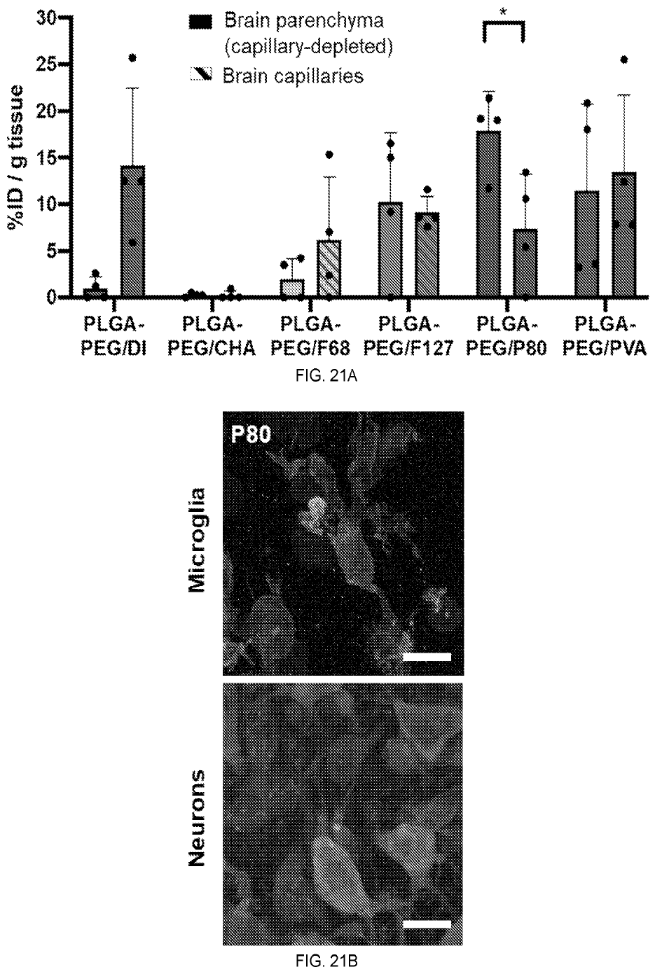

Individual trajectories were then analyzed for geometric features to distinguish between subtypes of diffusive transport. Characterization of the <MSD> curves showed that PLGA/F127 trajectories exhibited subdiffusive behavior more frequently than any other formulation, while the PLGA-PEG/DI and PLGA-PEG/CHA formulations were more likely to demonstrate superdiffusive behavior (FIG. 20A). These results were closely aligned with extraction of a, the anomalous diffusion coefficient, for each nanoparticle trajectory. The plurality of PLGA/F127 trajectories were classified as subdiffusive (α<1) while most PLGA-PEG/DI and PLGA-PEG/CHA trajectories had normal (α=1) diffusive behavior (FIG. 20B). Average trappedness, a geometric feature which describes the probability of the nanoparticle being trapped within a given radius, was highest for the PLGA/F127 nanoparticles and lowest for PLGA-PEG/DI nanoparticles (FIG. 20C). Conversely, PLGA/F127 nanoparticles were least efficient—that is, each time step resulted in small net displacements—while PLGA-PEG/DI nanoparticles were most efficient (FIG. 20D). For each geometric feature, all formulations with surfactants existed on a continuum between the immobilized PLGA/F127 nanoparticles and the diffusive PLGA-PEG/DI nanoparticles. Therefore, the results of multiple particle tracking (MPT) indicate that the inclusion of nonionic surfactants in nanoparticle formulations will increase the likelihood of cellular interaction—indicated by increasing subdiffusion, trappedness, and decreasing efficiency—at short length and time scales.

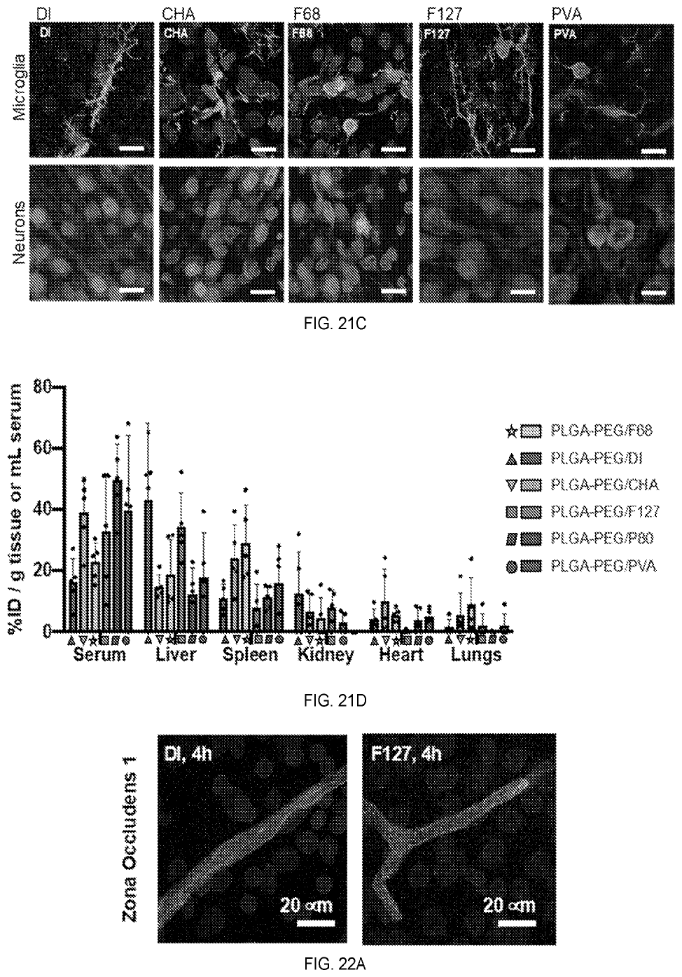

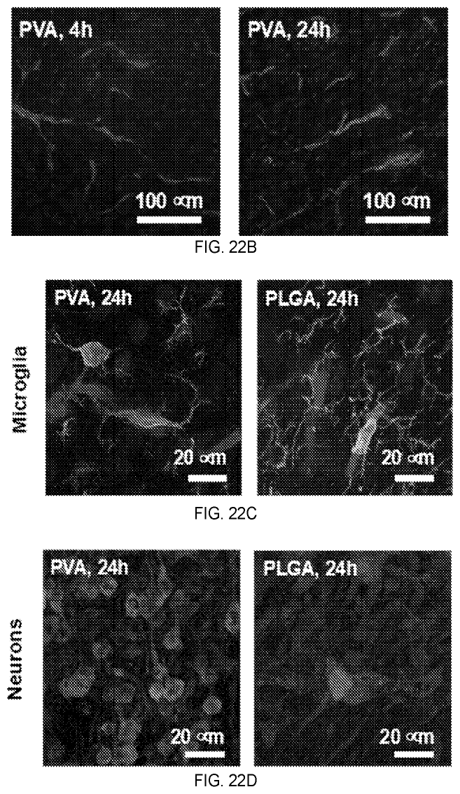

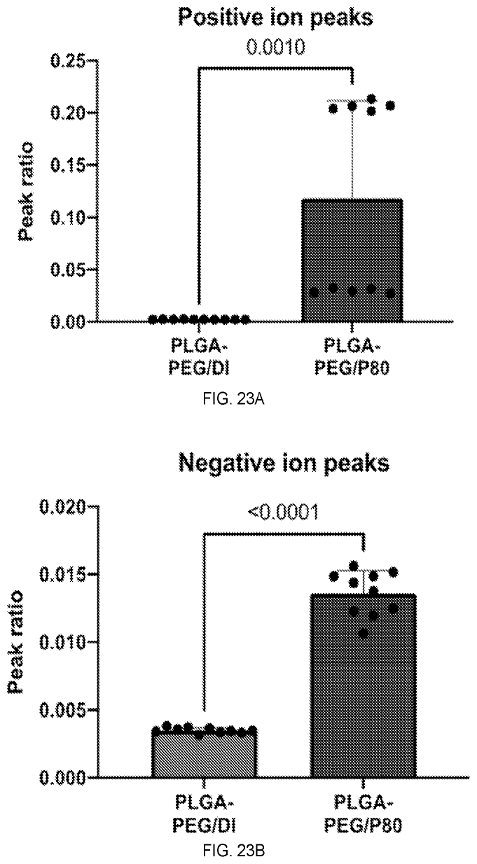

Nonionic surfactants enhance BBB permeation and accumulation in the brain. Beyond diffusion and cellular internalization, nanoparticle fate in the brain is dependent on favorable circulation kinetics, biodistribution, and transport across the BBB. These outcomes must be assessed in vivo. Each PLGA-PEG formulation was administered in healthy rat pups by intravenous tail vein injection. Four hours after administration, only PLGA-PEG/P80 nanoparticles were able to extravasate across the healthy BBB and uptake in neurons and microglia (FIGS. 21A-21C). To quantify nanoparticle penetration across the BBB, a capillary depletion technique was used on homogenized brain tissue which separated brain capillaries from the parenchyma (Banks, et al., J J Virol 2001, 75 (10), 4681-91). Results are presented as the percent of injected dose (% ID) per gram brain parenchyma or brain capillary (FIG. 21A). Only PLGA-PEG/P80 nanoparticles achieved a significantly higher concentration in the brain parenchyma compared to brain capillaries (17.8 vs 7.4% ID per g tissue, p=0.028). However, all nonionic surfactants improved BBB permeation compared to the no-surfactant control: without a surfactant, nanoparticle concentration in brain capillaries was 14.9-fold higher than in the parenchyma; F68, F127, P80, and PVA reduced this value to 3.2-, 0.9-, 0.4-, and 1.2-fold, respectively. Interestingly, the formulation with anionic CHA demonstrated nearly no accumulation in either the brain capillaries or parenchyma.

The fate of nanoparticles once within the brain parenchyma was investigated. Using confocal imaging, evidence of PLGA-PEG/P80 uptake was found in microglia and neurons within the hippocampus (FIG. 21B). In contrast, PLGA-PEG nanoparticles formulated without surfactant or with CHA, F68, F127, or PVA showed greater association with blood vessel structures (FIG. 21C). Staining of tight junction protein zona occludens 1 (ZO-1) confirmed these regions as brain capillaries (FIG. 22A). The accumulation of nanoparticles at the healthy BBB persists at least for 24 hours: patterns of PLGA-PEG/PVA nanoparticle localization in brain capillaries appear consistent at both 4 hours and 24 hours after administration, with no visible additional neuronal or microglial uptake of nanoparticles (FIGS. 22B-22D). Comparison with the PLGA/F127 formulation after 24 hours indicates that the absence of a PEG layer does not enable increased BBB transcytosis or cellular uptake (FIGS. 22C, 22D). Finally, analysis of nanoparticle biodistribution across serum and major organs supports that a large fraction of nanoparticles is still in circulation at 4 h. The liver and spleen also demonstrate nanoparticle accumulation, while more limited uptake was found in the kidney, heart, and lungs.

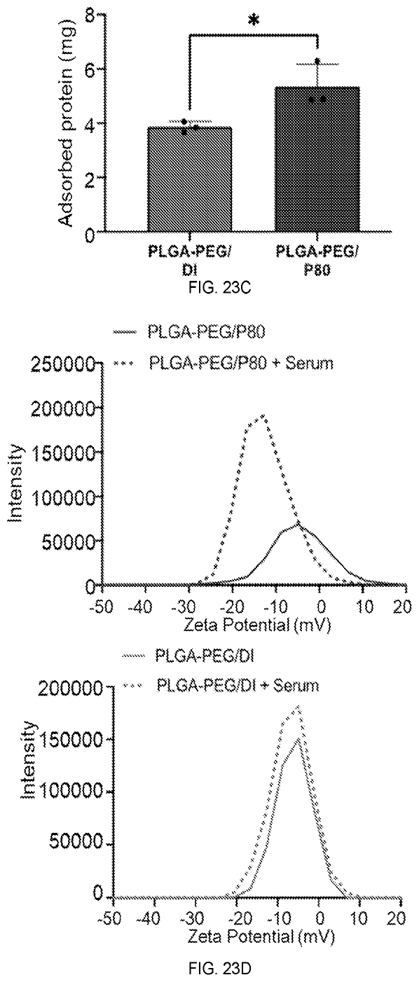

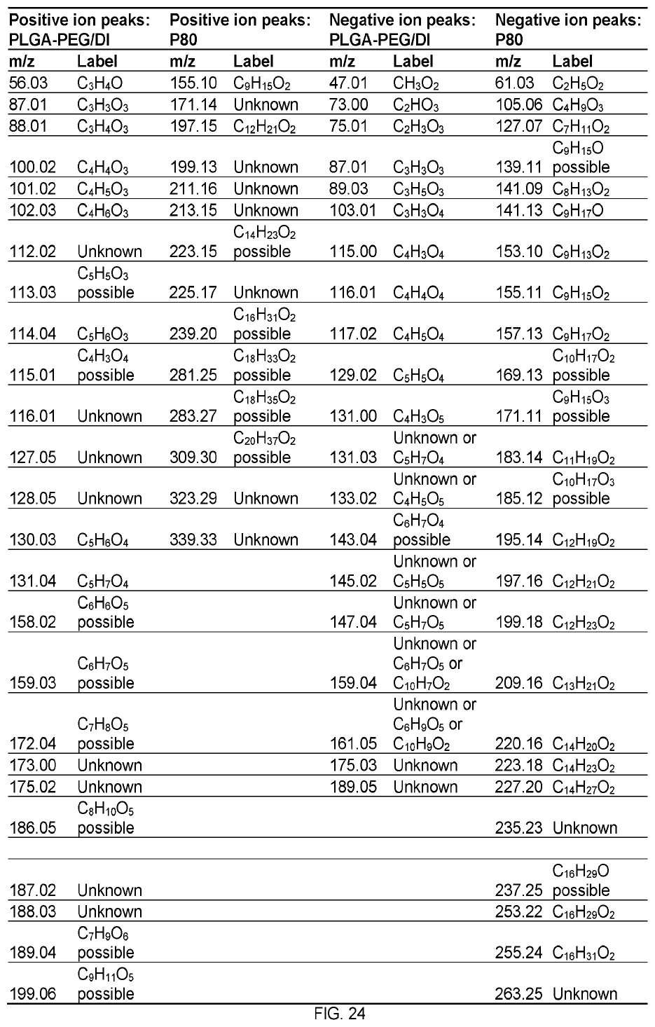

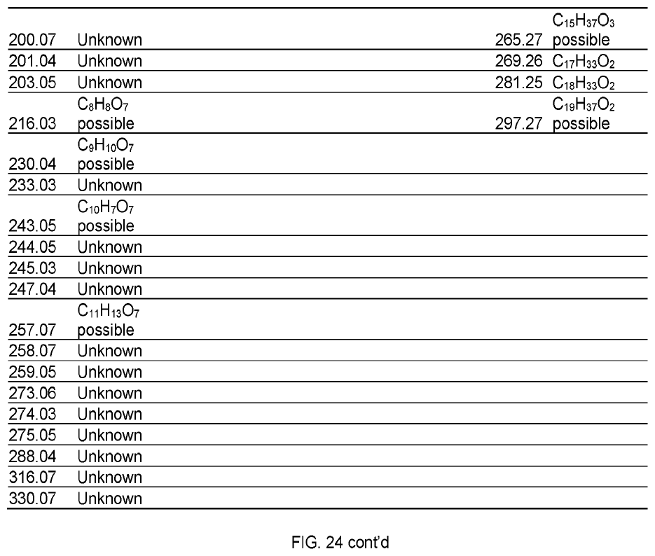

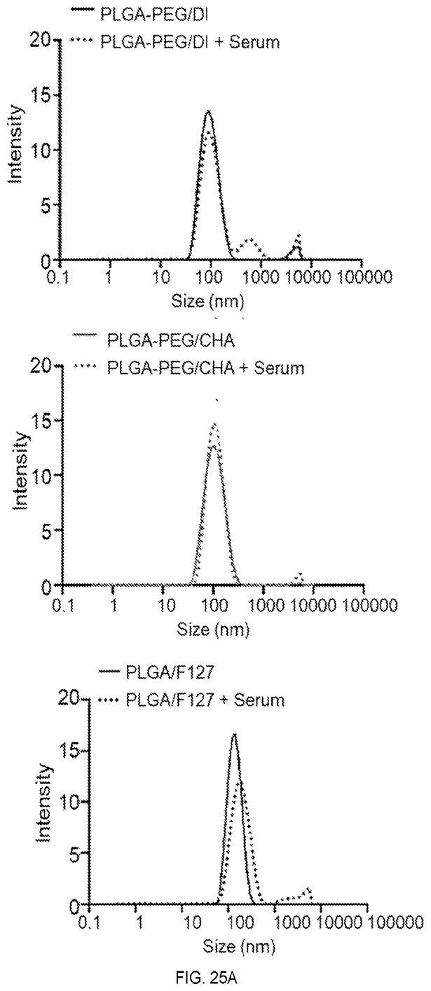

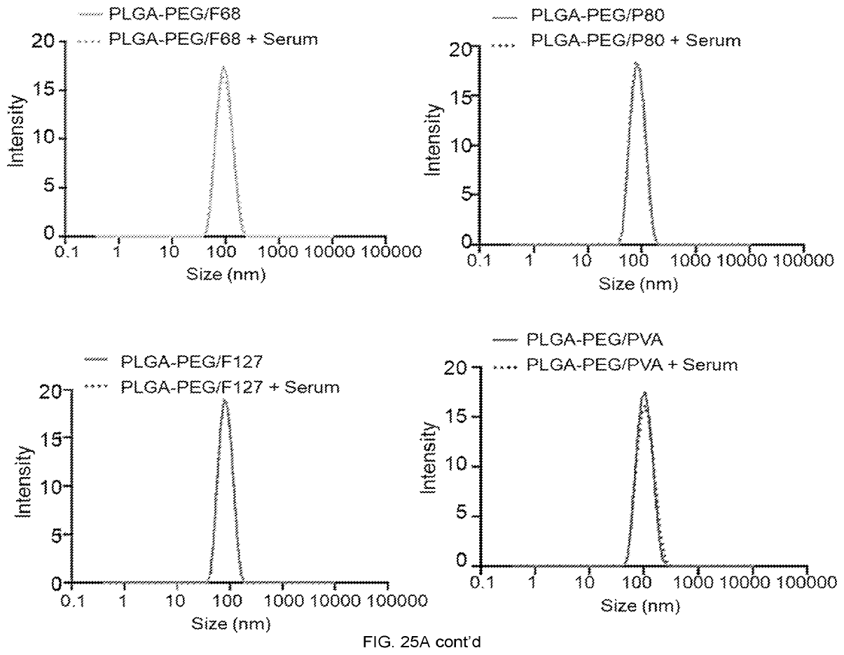

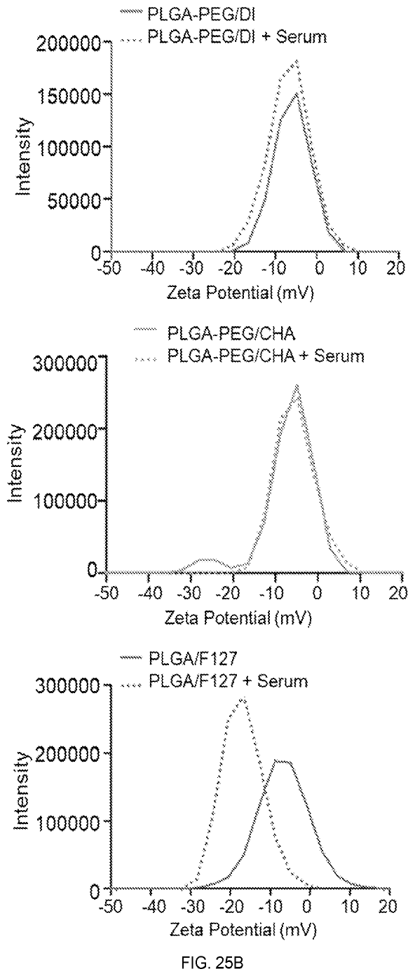

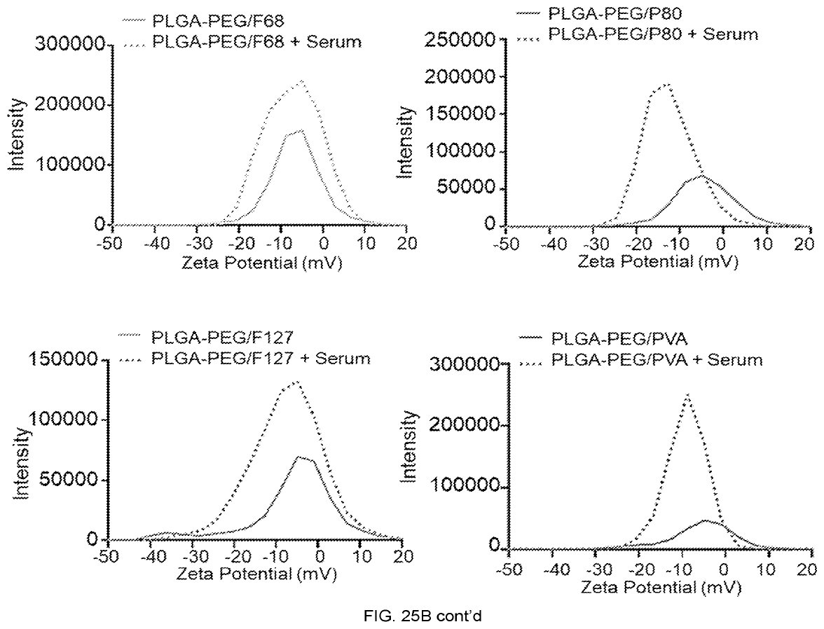

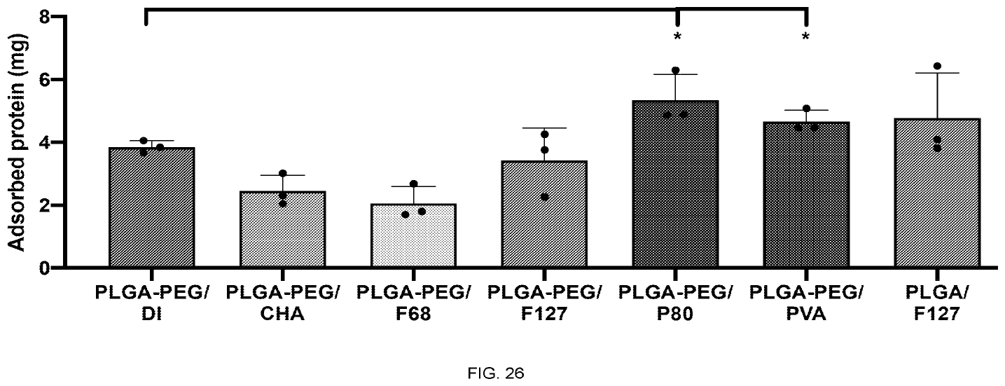

Polysorbate 80 is surface-associated and influences serum protein adsorption. Surfactants, including P80, may achieve favorable nanoparticle fate in the brain by facilitating plasma protein adsorption or desorption to the nanoparticle surface as the “protein corona” biolayer develops (Cox, et al., J Acs Nano 2018, 12 (7), 7292-7300). However, this phenomenon is poorly studied for nanoparticles with both PEG and surfactants. Time-of-flight secondary ion mass spectrometry was first used to establish that P80 is present on the nanoparticle surface. Positive and negative ion control spectra were taken from PLGA-PEG/DI nanoparticles and the P80 surfactant to determine unique peaks for each material. These peaks were then used to create a peak ratio A/(A+B) where A=sum of all P80 peaks and B=sum of all nanoparticle peaks. FIGS. 23A and 20B show the positive and negative ion peak ratios respectively from the ToF-SIMS data. FIG. 24 shows the proposed chemical identifications of the selected peaks for each material. As seen in FIGS. 23A and 23B, the PLGA-PEG/P80 nanoparticles show a higher relative intensity of the P80 peaks relative to the PLGA-PEG/DI nanoparticles (p=0.001 and p<0.0001 for the positive and negative ion peaks, respectively) confirming the presence of P80. Next PLGA-PEG/DI and PLGA-PEG/P80 nanoparticles were incubated in rat plasma for four hours at 37° C. and subsequently quantified the amount of plasma proteins adsorbed to the nanoparticles (FIG. 23C). The results show significantly increased levels of protein adsorption in the PLGA-PEG/P80 (p=0.0382) compared to nanoparticles without surfactant. Further evidence was found that elevated serum protein adsorption alters the nanoparticle surface charge: after the serum incubation, a significant negative shift in ζ-potential was observed for PLGA-PEG/P80 nanoparticles (−3.6 to −11.2 mV, p=0.0002) but not for PLGA-PEG/DI nanoparticles (FIG. 23D). This shift was not associated with a change in nanoparticle stability or polydispersity (FIG. 25A) and is larger than the shift experienced by any other surfactant-formulated nanoparticle (FIG. 25B). For comparison, quantification of serum protein adsorption on the other surfactant-formulated nanoparticles is presented in FIG. 26.

Conclusion. Results show that the incorporation of surfactant molecules in PLGA-PEG formulations enhances nanoparticle interaction within the brain microenvironment. Nonionic surfactants mediate enhanced BBB penetration after intravenous administration: PLGA-PEG/F68, PLGA-PEG/F127, PLGA-PEG/PVA, and PLGA-PEG/P80 nanoparticles exhibit 2-11-, 12-, and 19-fold greater uptake in the brain parenchyma, respectively, compared to PLGA-PEG nanoparticles without surfactant. Decreased microglial uptake ex vivo and hindered diffusive transport of nanoparticles containing surfactants were observed, demonstrating the role of surfactants in increasing short-term interactions with cells while reducing longer-term uptake processes. Charged surfactants, such as the anionic CHA, enable faster overall nanoparticle diffusion likely due to electrostatic interactions with brain ECM components. Overall, the presence of a surfactant influences the ability of nanoparticles to overcome biological barriers in the brain.

### Experimental Example 3. Enzyme Complexation with Hydrophobic Ion

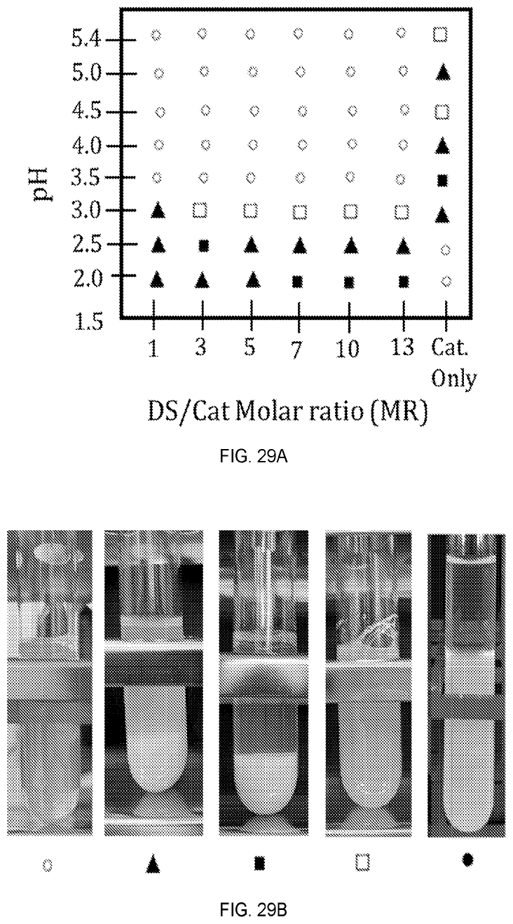

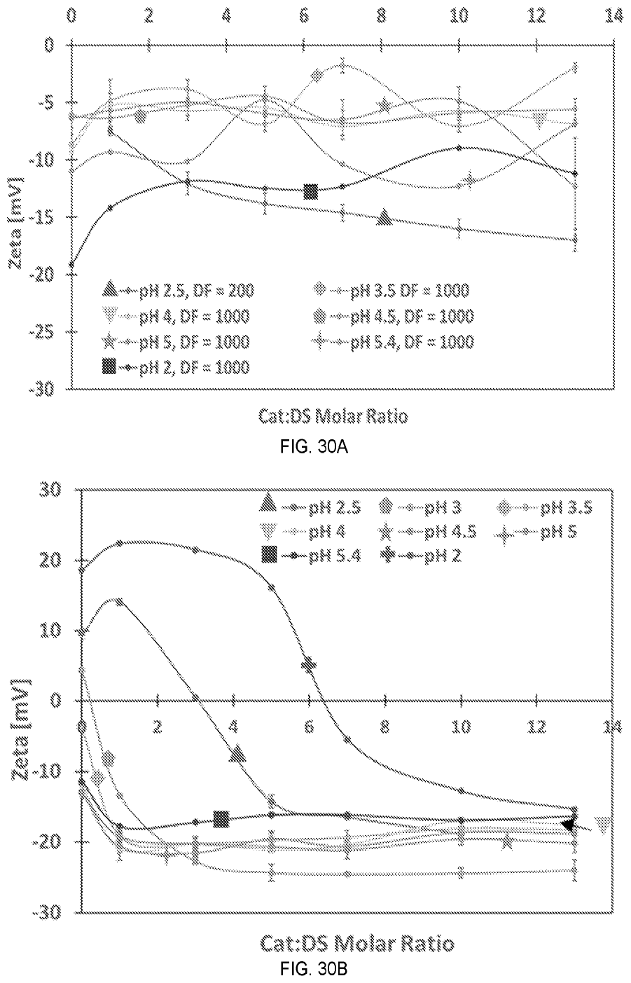

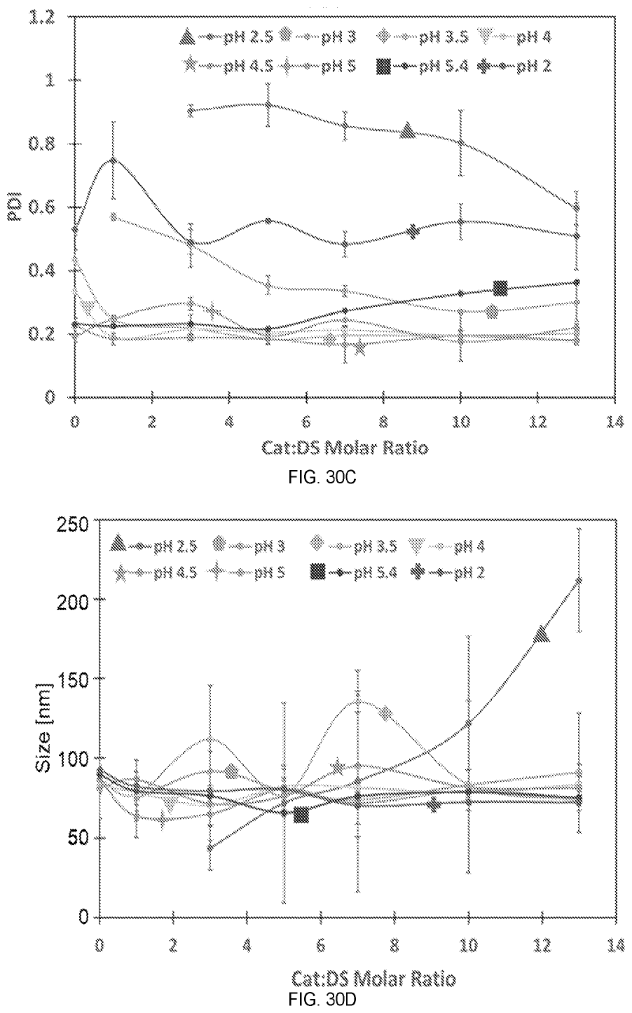

Catalase and dextran sulfate (DS) were mixed at different molar ratios and pHs to test conditions necessary for hydrophobic ion pairing (HIP) complex formation. HIP complex formation is considered successful when an insoluble complex is formed. HIP complexes formed at all molar ratios of DS to catalase but only at certain pHs. HIP complexes formed at pH 2, 2.5, and 3 at a DS/catalase molar ratio of 1 and at pH 2 and pH 2.5 at DS/catalase molar ratios of 3, 5, 7, 10, and 13 (FIG. 29A). The DS/Catalase complexes were further characterized for zeta-potential (mV) of complexes formed at different pHs, diluted 1000-fold in 10 mM NaCl (FIG. 30A), zeta-potential of complexes formed at different pHs at original solution volume (FIG. 30B), polydispersity (PDI) of complexes at different pHs (FIG. 30C), and size (nm) of complexes at different pHs. (FIG. 30D).

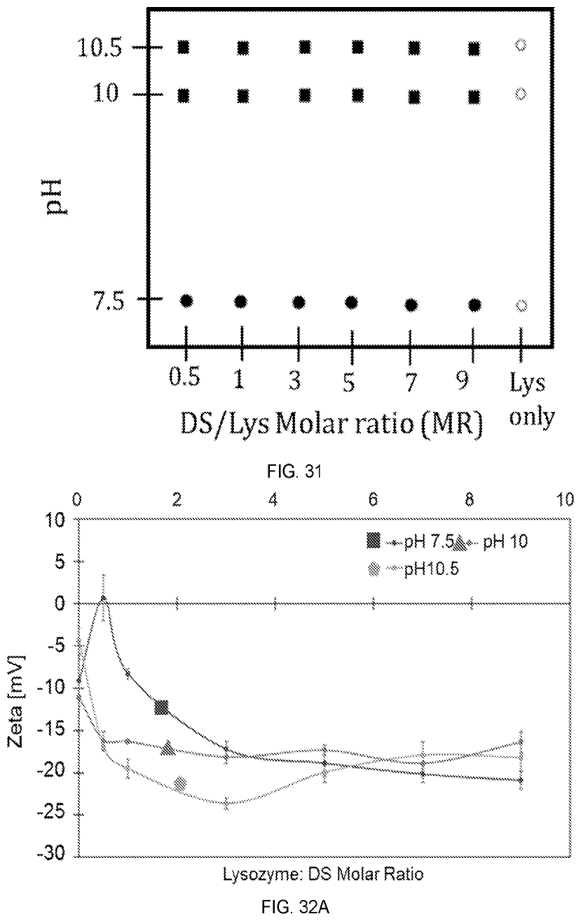

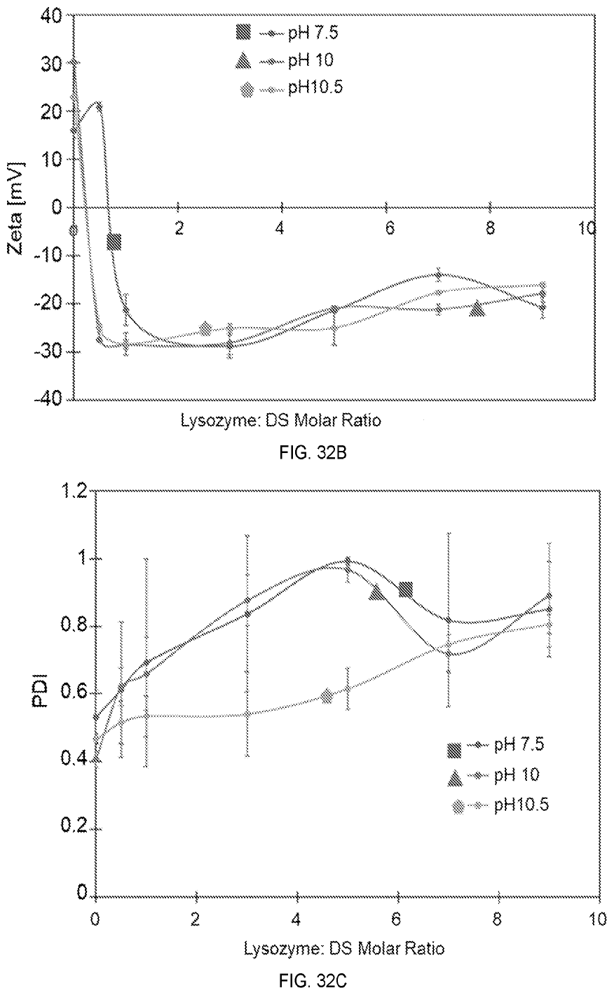

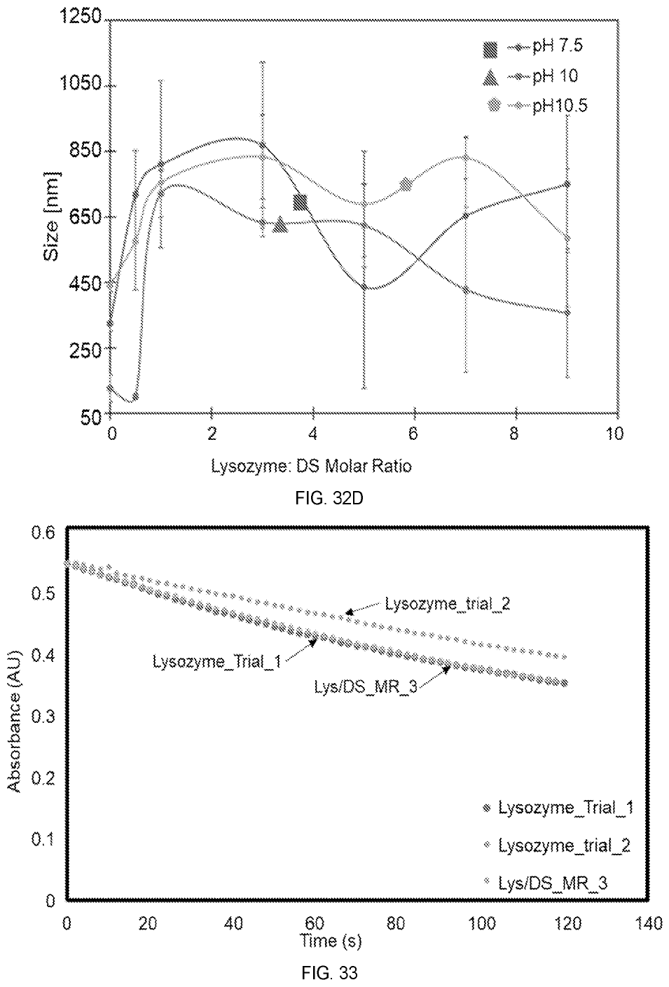

HIP complexes were also formed using DS and lysozyme at varying pH and molar ratio. HIP complexes formed at all tested molar ratios of DS to lysozyme including 0.5, 1, 3, 5, 7, and 9. Strong HIP complexes formed at pH10 and pH10.5 at these molar ratios (FIG. 31). The DS/lysozyme complexes were further characterized for zeta-potential (mV) of complexes formed at different pHs, diluted 1000-fold in 10 mM NaCl (FIG. 32A), zeta-potential of complexes formed at different pHs at original solution volume (FIG. 32B), polydispersity (PDI) of complexes at different pHs (FIG. 32C), and hydrodynamic diameter (nm) of complexes at different pHs (FIG. 32D). Activity of lysozyme is also shown over time at pH7.46 once complexed with dextran sulfate in FIG. 33.

(xi) Closing Paragraphs. Unless otherwise indicated, the practice of the present disclosure can employ conventional techniques of chemistry, organic chemistry, biochemistry, analytical chemistry, and physical chemistry. These methods are described in the following publications. See, e.g., Harcourt, et al., Holt McDougal Modern Chemistry: Student Edition (2018); J. Karty, Organic Chemistry Principles and Mechanisms (2014); Nelson, et al., Lehninger Principles of Biochemistry 5th edition (2008); Skoog, et al., Fundamentals of Analytical Chemistry (8th Edition); Atkins, et al., Atkins' Physical Chemistry (11th Edition).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of preparing a hydrophobic ion pairing complex for nanoparticle formation, the method comprising:
mixing, within a solution, dextran sulfate and an enzyme to form a hydrophobic ion pairing complex within the solution;
centrifuging the solution to separate the hydrophobic ion pairing complex from the solution; and
collecting the separated hydrophobic ion pairing complex such that the enzyme retains activity,
thereby preparing the hydrophobic ion pairing complex for nanoparticle formation.

2. A method of preparing enzyme-loaded nanoparticles, the method comprising:
mixing, within a solution, dextran sulfate and an enzyme to form a hydrophobic ion pairing complex within the solution;
centrifuging the solution to separate the hydrophobic ion pairing complex from the solution;
collecting the separated hydrophobic ion pairing complex such that the enzyme retains activity;
mixing a polymer with the collected hydrophobic ion pairing complex within a solvent to form a mixture;
adding the mixture to an aqueous solution to form the enzyme-loaded nanoparticles; and
separating the formed enzyme-loaded nanoparticles from the aqueous solution, thereby preparing enzyme-loaded nanoparticles.

3. A method of preparing a hydrophobic ion pairing complex, the method comprising:
mixing, within a solution, a hydrophobic ion and a first enzyme to form a hydrophobic ion pairing complex; and
separating the hydrophobic ion pairing complex from the solution,
thereby preparing the hydrophobic ion pairing complex,
wherein the hydrophobic ion comprises dextran sulfate, taurocholic acid, sodium dodecyl sulfate, 1-hydroxy-2-naphthoic acid, 2-naphthalene sulfonic acid, alginic acid, arginine-hexadecanoyl ester, arginine-nonyl ester, benethamine, brilliant blue FCF, cetrimonium bromide (CTAB), chitosan, chlorhexidine, cholesteryl hemisuccinate, cholic acid, CM-PEG56, dimyristoyl phosphatidyl glycerol, dioleoyl phosphatidic acid (DOPA), docosahexaenoic acid, docusate sodium, hexadecylphosphate, hyaluronic acid, laurylamine, linoleic acid, losartan, maprotiline, N α-deoxycholyl-L-lysyl-methylester, N,N′-dibenzylethylene diamine (DBDA), N,N-dimethyldodecyl amine (DDA), N,N-dimethylhexyl amine, N,N-dimethyloctadecyl amine, N,N-dipalmitoyl-L-lysine, oleic acid, pamoic acid, sodium acetate, sodium alginate, sodium cholesteryl sulfate, sodium decanesulfonate, sodium decanoate, sodium deoxycholate, sodium docusate, sodium dodecyl benzenesulfonate, sodium laurate, sodium oleate, sodium stearate, sodium stearoyl glutamate, sodium stearyl sulfate, sodium taurodeoxycholate, sodium tetradecyl sulfate, sodium tripolyphosphate, stearylamine, tetrabutylammonium bromide, tetraheptylammonium bromide, tetrahexylammonium bromide, tetraoctylammonium bromide, tetrapentylammonium bromide, triethylamine, or vitamin E succinate;
and wherein the enzyme retains activity within the hydrophobic ion pairing complex.

4. The method of claim 3, wherein the mixing comprises vortexing.

5. The method of claim 3, wherein the separating uses centrifugation.

6. The method of claim 3, wherein the first enzyme has a molecular weight between 10 kDa and 300 kDa.

7. The method of claim 3, wherein the first enzyme has a molecular weight of 10 kDa to 50 kDa.

8. The method of claim 3, wherein the first enzyme has a molecular weight of 14 kDa.

9. The method of claim 3, wherein the first enzyme has a molecular weight of 50 kDa to 150 kDa.

10. The method of claim 3, wherein the first enzyme has a molecular weight of 150 kDa to 300 kDa.

11. The method of claim 3, wherein the first enzyme has a molecular weight of 240 kDa.

12. The method of claim 3, wherein the first enzyme comprises catalase, lysozyme, chymotrypsin, trypsin, serratiopeptidase, bromelain, ficin, papain, serrapeptase, superoxide dismutase (SOD), glutamate cysteine ligase (GCL), or glutathione synthase (GS).

13. The method of claim 3, wherein the first enzyme comprises catalase.

14. The method of claim 3, wherein the first enzyme comprises lysozyme.

15. The method of claim 3, wherein the first enzyme comprises superoxide dismutase (SOD), catalase (CAT), glutathione peroxidase (GPx), aldehyde oxidase, glyoxalase, myeloperoxidase, nitric oxide synthase, and/or sphingomyelin phosphodiesterase.

16. The method of claim 3, wherein the first enzyme comprises glucocerebrosidase, sphingomyelin cholinephosphohydrolase, acid sphingomyelinase, acid alpha-glucosidase, aspartylglucosaminidase, alpha-galactosidase A, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, and/or acid lipase.

17. The method of claim 3, wherein the first enzyme comprises glucose-6-phosphatase, fructose-1,6-bisphosphatase, tibose-phosphate isomerase, transaldolase, succinate dehydrogenase, glucose-6-phosphate dehydrogenase, fumarase, pyruvate dehydrogenase complex, and/or N-acetylglutamate synthetase.

18. The method of claim 3, wherein the first enzyme retains activity following administration to a subject.

19. The method of claim 3, wherein the first enzyme retains 60%, 65%, 70%, 75%, 80%, 85%, 90%, 95%, or 100% activity compared to a benchmark enzyme activity.

20. The method of claim 19, wherein the benchmark enzyme activity is determined by active units per mass according to the enzyme's manufacturer.

21. The method of claim 3, further comprising mixing a second enzyme within the solution.

22. The method of claim 21, wherein the second enzyme is catalase.

23. The method of claim 21, wherein the second enzyme is lysozyme.

24. The method of claim 21, wherein at least one of the first enzyme or the second enzyme is an anti-inflammatory enzyme, an excitotoxicity-altering enzyme, or an antioxidative enzyme.

25. The method of claim 24, wherein the anti-inflammatory enzyme comprises chymotrypsin, trypsin, serratiopeptidase, bromelain, ficin, papain, or serrapeptase.

26. The method of claim 24, wherein the excitotoxicity-altering enzyme comprises glutamate decarboxylase, glutamate dehydrogenase, pyrroline-5-carboxylate synthase (P5CS), serum glutamate oxaloacetate transaminase (AST), or serum glutamate pyruvate transaminase (ALT).

27. The method of claim 24, wherein the antioxidative enzyme comprises superoxide dismutase (SOD), glutamate cysteine ligase (GCL), or glutathione synthase (GS).

28. The method of claim 3, wherein the solution has a pH ranging from pH 2.0 to 10.5.

29. The method of claim 3, wherein the solution is a buffering agent comprising a phosphate buffer, a citrate buffer, or an acetate buffer.

30. The method of claim 3, wherein the solution is a phosphate buffer.

31. The method of claim 3, wherein the hydrophobic ion is dextran sulfate.

32. The method of claim 3, wherein the method further comprises lyophilizing the hydrophobic ion pairing complex.

33. The method of claim 3, wherein the method further comprises incorporating the hydrophobic ion pairing complex into a polymeric nanoparticle.

34. The method of claim 3, wherein the hydrophobic ion pairing complex has a molar ratio of at least 0.5 of hydrophobic ion to enzyme.

35. The method of claim 3, wherein the hydrophobic ion pairing complex has a hydrophobic ion to enzyme charge ratio of 1:1.

36. A method of preparing an enzyme-loaded nanoparticle, the method comprising:
mixing a polymer with a hydrophobic ion pairing complex within a solvent to form a mixture, wherein the hydrophobic ion pairing complex comprises an enzyme and a hydrophobic ion;
adding the mixture to an aqueous solution to form the enzyme-loaded nanoparticles; and
separating the formed enzyme-loaded nanoparticles from the aqueous solution, thereby preparing enzyme-loaded nanoparticles.

37. The method of claim 36, wherein the hydrophobic ion pairing complexes are prepared according to the method of claim 3.

38. The method of claim 36, wherein the polymer comprises a hydrophobic polymer.

39. The method of claim 36, wherein the polymer comprises a hydrophobic polymer and polyethylene glycol (PEG).

40. The method of claim 39, wherein the hydrophobic polymer comprises polylactic co-glycolic acid (PLGA), polylactic acid (PLA), or poly-ε-caprolactone (PCL), ethyl cellulose (EC), polybutylcyanoacrylate (PBCA), polypropylene oxide (PPO), or polyhydroxybutyrate (PHB).

41. The method of claim 39, wherein the hydrophobic polymer is polylactic co-glycolic acid (PLGA).

42. The method of claim 36, wherein the hydrophobic ion is dextran sulfate.

43. The method of claim 36, wherein the enzyme is catalase, lysozyme, chymotrypsin, trypsin, serratiopeptidase, bromelain, ficin, papain, serrapeptase, superoxide dismutase (SOD), glutamate cysteine ligase (GCL), or glutathione synthase (GS).

44. The method of claim 36, wherein the enzyme is catalase.

45. The method of claim 36, wherein the enzyme is lysozyme.

46. The method of claim 36, wherein the enzyme comprises superoxide dismutase (SOD), catalase (CAT), glutathione peroxidase (GPx), aldehyde oxidase, glyoxalase, myeloperoxidase, nitric oxide synthase, and/or sphingomyelin phosphodiesterase.

47. The method of claim 36, wherein the enzyme comprises glucocerebrosidase, sphingomyelin cholinephosphohydrolase, acid sphingomyelinase, acid alpha-glucosidase, aspartylglucosaminidase, alpha-galactosidase A, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, and/or acid lipase.

48. The method of claim 36, wherein the enzyme comprises glucose-6-phosphatase, fructose-1,6-bisphosphatase, tibose-phosphate isomerase, transaldolase, succinate dehydrogenase, glucose-6-phosphate dehydrogenase, fumarase, pyruvate dehydrogenase complex, and/or N-acetylglutamate synthetase.

49. The method of claim 36, wherein the enzyme comprises a first enzyme and a second enzyme.

50. The method of claim 49, wherein the second enzyme is an anti-inflammatory enzyme, an excitotoxicity-altering enzyme, or an antioxidative enzyme.

51. The method of claim 50, wherein the anti-inflammatory enzyme comprises chymotrypsin, trypsin, serratiopeptidase, bromelain, ficin, papain, or serrapeptase.

52. The method of claim 50, wherein the excitotoxicity-altering enzyme comprises enzymes that perform glutamate catalysis.

53. The method of claim 50, wherein the antioxidative enzyme comprises superoxide dismutase (SOD), glutamate cysteine ligase (GCL), or glutathione synthase (GS).

54. The method of claim 36, wherein the solvent is a water-miscible organic solvent.

55. The method of claim 54, wherein the water-miscible organic solvent comprises acetone, acetonitrile, dimethylsulfoxide (DMSO), methanol, acetaldehyde, acetic acid, 1,2-Butanediol, 1,3-Butanediol, 1,4-Butanediol, 2-Butoxyethanol, butyric acid, dethanolamine, diethylenetriamine, dimethylformamide, dimethoxyethane, 1,4-Dioxane, ethanol, ethylamine, ethylene glycol, formic acid, furfuryl alcohol, glycerol, methyl diethanolamine, methyl isocyanide, N-Methyl-2-pyrrolidone, 1-Propanol, 1,3-Propanediol, 1,5-Pentanediol, 2-Propanol, propanoic acid, propylene glycol, pyridine, tetrahydrofuran, or triethylene glycol.

56. The method of claim 54, wherein the water-miscible organic solvent is acetone.

57. The method of claim 36, wherein the mixing comprises vortexing or stirring.

58. The method of claim 36, wherein the adding the mixture to the aqueous solution comprises adding the mixture dropwise into the aqueous solution and stirring.

59. The method of claim 36, wherein the aqueous solution comprises a surfactant in solution.

60. The method of claim 59, wherein the surfactant comprises a polysorbate, a poloxamer, a nonionic surfactant, or an anionic surfactant.

61. The method of claim 60, wherein the polysorbate is polysorbate 80.

62. The method of claim 60, wherein the poloxamer is poloxamer 188 (F68) or poloxamer 407 (F127).

63. The method of claim 60, wherein the nonionic surfactant is polyvinyl alcohol (PVA).

64. The method of claim 60, wherein the anionic surfactant is cholic acid.

65. The method of claim 36, wherein the hydrophobic ion pairing complex is a solid and/or is dissolved in a water miscible organic solvent.

66. The method of claim 65, wherein the water-miscible organic solvent comprises acetone, acetonitrile, dimethylsulfoxide (DMSO), methanol, acetaldehyde, acetic acid, 1,2-Butanediol, 1,3-Butanediol, 1,4-Butanediol, 2-Butoxyethanol, butyric acid, dethanolamine, diethylenetriamine, dimethylformamide, dimethoxyethane, 1,4-Dioxane, ethanol, ethylamine, ethylene glycol, formic acid, furfuryl alcohol, glycerol, methyl diethanolamine, methyl isocyanide, N-Methyl-2-pyrrolidone, 1-Propanol, 1,3-Propanediol, 1,5-Pentanediol, 2-Propanol, propanoic acid, propylene glycol, pyridine, tetrahydrofuran, or triethylene glycol.

67. The method of claim 36, wherein the solvent is a water-immiscible organic solvent.

68. The method of claim 67, wherein the water-immiscible organic solvent comprises dichloromethane, chloroform, or acyl acetate.

69. The method of claim 67, wherein the water-immiscible organic solvent comprises dichloromethane.

70. The method of claim 36, wherein the mixing comprises homogenization or sonication.

71. The method of claim 36, wherein the adding the mixture to the aqueous solution comprises pouring the mixture into the aqueous solution and stirring.

72. The method of claim 36, wherein the aqueous solution comprises a surfactant in solution.

73. The method of claim 72, wherein the surfactant comprises a polysorbate, a poloxamer, a nonionic surfactant, or an anionic surfactant.

74. The method of claim 73, wherein the polysorbate is polysorbate 80.

75. The method of claim 73, wherein the poloxamer is poloxamer 188 (F68) or poloxamer 407 (F127).

76. The method of claim 73, wherein the nonionic surfactant is polyvinyl alcohol (PVA).

77. The method of claim 73, wherein the anionic surfactant is cholic acid.

78. The method of claim 36, wherein the separating of the nanoparticles is by centrifuging.

79. The method of claim 36, wherein the separating of the nanoparticles is by washing and ultracentrifugation with a buffer.

80. The method of claim 79, wherein the buffer is phosphate buffer.

81. The method of claim 36, further comprising resuspending the separated nanoparticles in deionized water.

82. A nanoparticle comprising (i) a polymer and (ii) a hydrophobic ion pairing complex comprising a hydrophobic ion and an enzyme.

83. The nanoparticle of claim 82, wherein the polymer comprises a hydrophobic polymer.

84. The nanoparticle of claim 82, wherein the polymer comprises the hydrophobic polymer and a polyethylene glycol (PEG).

85. The nanoparticle of claims 83 or 84, wherein the hydrophobic polymer comprises polylactic co-glycolic acid (PLGA), polylactic acid (PLA), polyglycolic acid (PGA), poly-ε-caprolactone (PCL), ethyl cellulose (EC), polybutylcyanoacrylate (PBCA), polypropylene oxide (PPO), or polyhydroxybutyrate (PHB).

86. The nanoparticle of claim 85, wherein the hydrophobic polymer is polylactic co-glycolic acid (PLGA).

87. The nanoparticle of claim 82, wherein the polymer comprises PLGA and PEG, the hydrophobic ion comprises dextran sulfate, and the enzyme comprises catalase.

88. The nanoparticle of claim 82, wherein the polymer comprises PLGA and PEG, the hydrophobic ion comprises dextran sulfate, and the enzyme comprises lysozyme.

89. The nanoparticle of claim 82, wherein the enzyme has a molecular weight ranging between 10 kDa and 300 kDa.

90. The nanoparticle of claim 82, wherein the enzyme has a molecular weight of 10 kDa to 50 kDa.

91. The nanoparticle of claim 82, wherein the enzyme has a molecular weight of 14 kDa.

92. The nanoparticle of claim 82, wherein the enzyme has a molecular weight of 50 kDa to 150 kDa.

93. The nanoparticle of claim 82, wherein the enzyme has a molecular weight of 150 kDa to 300 kDa.

94. The nanoparticle of claim 82, wherein the enzyme has a molecular weight of 240 kDa.

95. The nanoparticle of claim 82, wherein the enzyme is positively charged at a pH when the hydrophobic ion is negatively charged.

96. The nanoparticle of claim 95, wherein the pH is 2.0.

97. The nanoparticle of claim 95, wherein the pH is 10 to 10.5.

98. The nanoparticle of claim 82, wherein the enzyme retains activity following administration to a subject.

99. The nanoparticle of claim 82, wherein the enzyme retains 60%, 65%, 70%, 75%, 80%, 85%, 90%, 95%, or 100% activity compared to a benchmark enzyme activity.

100. The nanoparticle of claim 99, wherein the benchmark enzyme activity is determined by active units per mass according to the enzyme's manufacturer.

101. The nanoparticle of claim 82, wherein the enzyme is catalase, lysozyme, chymotrypsin, trypsin, serratiopeptidase, bromelain, ficin, papain, serrapeptase, superoxide dismutase (SOD), glutamate cysteine ligase (GCL), or glutathione synthase (GS).

102. The nanoparticle of claim 82, wherein the enzyme is catalase.

103. The nanoparticle of claim 82, wherein the enzyme is lysozyme.

104. The nanoparticle of claim 82, wherein the enzyme comprises superoxide dismutase (SOD), catalase (CAT), glutathione peroxidase (GPx), aldehyde oxidase, glyoxalase, myeloperoxidase, nitric oxide synthase, and/or sphingomyelin phosphodiesterase.

105. The nanoparticle of claim 82, wherein the enzyme comprises glucocerebrosidase, sphingomyelin cholinephosphohydrolase, acid sphingomyelinase, acid alpha-glucosidase, aspartylglucosaminidase, alpha-galactosidase A, palmitoyl protein thioesterase, tripeptidyl peptidase, lysosomal transmembrane protein, cysteine transporter, acid ceramidase, acid alpha-L-fucosidase, cathepsin A, acid beta-glucosidase, acid beta-galactosidase, iduronate-2-sulfatase, alpha-L-iduronidase, galactocerebrosidase, acid alpha-mannosidase, acid beta-mannosidase, arylsulfatase B, arylsulfatase A, N-acetylgalactosamine-6-sulfate, N-acetylglucosamine-1-phosphotransferase, acid sphingomyelinase, NPC-1, alpha-glucosidase, beta-hexosaminidase B, heparan N-sulfatase, alpha-N-acetylglucosaminidase, acetyl-CoA: alpha-glucosaminide, N-acetylglucosamine-6-sulfate, alpha-N-acetylgalactosaminidase, alpha-neuramidase, beta-glucuronidase, beta-hexosaminidase A, and/or acid lipase.

106. The nanoparticle of claim 82, wherein the enzyme comprises glucose-6-phosphatase, fructose-1,6-bisphosphatase, tibose-phosphate isomerase, transaldolase, succinate dehydrogenase, glucose-6-phosphate dehydrogenase, fumarase, pyruvate dehydrogenase complex, and/or N-acetylglutamate synthetase.

107. The nanoparticle of claim 82, wherein the enzyme comprises at least two enzymes.

108. The nanoparticle of claim 107, wherein at least one of the at least two enzymes is catalase.

109. The nanoparticle of claim 107, wherein at least one of the at least two enzymes is lysozyme.

110. The nanoparticle of claim 107, wherein at least one of the at least two enzymes is an anti-inflammatory enzyme, an excitotoxicity-altering enzyme, or an antioxidative enzyme.

111. The nanoparticle of claim 110, wherein the anti-inflammatory enzyme comprises chymotrypsin, trypsin, serratiopeptidase, bromelain, ficin, papain, or serrapeptase.

112. The nanoparticle of claim 110, wherein the excitotoxicity-altering enzyme comprises glutamate decarboxylase, glutamate dehydrogenase, pyrroline-5-carboxylate synthase (P5CS), serum glutamate oxaloacetate transaminase (AST), or serum glutamate pyruvate transaminase (ALT).

113. The nanoparticle of claim 110, wherein the antioxidative enzyme comprises superoxide dismutase (SOD), glutamate cysteine ligase (GCL), or glutathione synthase (GS).

114. The nanoparticle of claim 82, wherein the hydrophobic ion comprises dextran sulfate, taurocholic acid, sodium dodecyl sulfate, 1-hydroxy-2-naphthoic acid, 2-naphthalene sulfonic acid, alginic acid, arginine-hexadecanoyl ester, arginine-nonyl ester, benethamine, brilliant blue FCF, cetrimonium bromide (CTAB), chitosan, chlorhexidine, cholesteryl hemisuccinate, cholic acid, CM-PEG56, dimyristoyl phosphatidyl glycerol, dioleoyl phosphatidic acid (DOPA), docosahexaenoic acid, docusate sodium, hexadecylphosphate, hyaluronic acid, laurylamine, linoleic acid, losartan, maprotiline, N α-deoxycholyl-L-lysyl-methylester, N,N′-dibenzylethylene diamine (DBDA), N,N-dimethyldodecyl amine (DDA), N,N-dimethylhexyl amine, N,N-dimethyloctadecyl amine, N,N-dipalmitoyl-L-lysine, oleic acid, pamoic acid, sodium acetate, sodium alginate, sodium cholesteryl sulfate, sodium decanesulfonate, sodium decanoate, sodium deoxycholate, sodium docusate, sodium dodecyl benzenesulfonate, sodium laurate, sodium oleate, sodium stearate, sodium stearoyl glutamate, sodium stearyl sulfate, sodium taurodeoxycholate, sodium tetradecyl sulfate, sodium tripolyphosphate, stearylamine, tetrabutylammonium bromide, tetraheptylammonium bromide, tetrahexylammonium bromide, tetraoctylammonium bromide, tetrapentylammonium bromide, triethylamine, or vitamin E succinate.

115. The nanoparticle of claim 82, wherein the hydrophobic ion is dextran sulfate.

116. The nanoparticle of claim 82, further comprising a surfactant.

117. The nanoparticle of claim 116, wherein the surfactant comprises a polysorbate, a poloxamer, a nonionic surfactant, or an anionic surfactant.

118. The nanoparticle of claim 117, wherein the polysorbate is polysorbate 80 (P80).

119. The nanoparticle of claim 117, wherein the poloxamer is poloxamer 188 (F68) or poloxamer 407 (F127).

120. The nanoparticle of claim 117, wherein the nonionic surfactant is polyvinyl alcohol (PVA).

121. The nanoparticle of claim 117, wherein the anionic surfactant is cholic acid.

122. The nanoparticle of claim 82, wherein the nanoparticle does not require cell targeting ligands.

123. A method of treating a condition comprising administering a therapeutically effective amount of the nanoparticle of claim 82 to a subject in need thereof.

124. The method of claim 123, wherein the condition is a neurological condition.

125. The method of claim 124, wherein the neurological condition is an acute neurological injury or a chronic neurodegenerative condition.

126. The method of claim 125, wherein the acute neurological injury is an encephalopathy.

127. The method of claim 126, wherein the encephalopathy is neonatal hypoxic-ischemic encephalopathy.

128. The method of claim 125, wherein the chronic neurodegenerative condition comprises multiple sclerosis, amyotrophic lateral sclerosis, Huntington's disease, Alzheimer's disease, or Parkinson's disease.

129. The method of claim 123, wherein the condition is a non-neurological condition.

130. The method of claim 129, wherein the non-neurological condition comprises oxidative stress burden, lysosomal storage disease, or a metabolic deficiency.

131. The method of claim 130, wherein the oxidative stress burden is due to type 2 diabetes.

132. The method of claim 123, wherein the subject is in need thereof following cardiac arrest.

133. The method of claim 123, wherein the administering is intravenous, intraperitoneal, intramuscular, oral, or nasal administration.

134. The method of claim 123, wherein the administering is intravenous.

135. The method of claim 123, wherein the subject is a mammal.

136. The method of claim 135, wherein the mammal is a human.

137. The method of claim 136, wherein the human is a neonate.

138. The method of claim 136, wherein the human is an infant or a child.

139. The method of claim 136, wherein the human is an adult.

140. The method of claim 123, wherein the enzyme retains activity following administration to a subject at least until a therapeutic objective is achieved.

141. The method of claim 123, wherein the enzyme retains 60%, 65%, 70%, 75%, 80%, 85%, 90%, 95%, or 100% activity compared to a benchmark enzyme activity.

142. The method of claim 141, wherein the benchmark enzyme activity is determined by active units per mass according to the enzyme's manufacturer.

143. A method of delivering an enzyme to a central nervous system of a subject comprising administering the nanoparticle of claim 82 to the subject.

144. The method of claim 143, wherein the administering comprises intravenous, intraperitoneal, intramuscular, oral, or nasal administration.

145. The method of claim 143, wherein the administering is intravenous.

146. The method of claim 143, wherein the enzyme retains activity following administration to the subject at least until a physiological effect is achieved.

147. The method of claim 146, wherein the physiological effect is reduced oxidative burden.

148. The method of claim 146, wherein the physiological effect is neuroprotection.

149. The method of claim 143, wherein the enzyme retains 60%, 65%, 70%, 75%, 80%, 85%, 90%, 95%, or 100% activity compared to a benchmark enzyme activity.

150. The method of claim 149, wherein the benchmark enzyme activity is determined by active units per mass according to the enzyme's manufacturer.

151. The method of claim 143, wherein the subject is a mammal.

152. The method of claim 151, wherein the mammal is a human.

153. The method of claim 152, wherein the human is a neonate.

154. The method of claim 152, wherein the human is an infant or a child.

155. The method of claim 152, wherein the human is an adult.
